(19) United States
(12) Patent Application Publication
(10) Pub. No.: US 2024/0325683 A1
(43) Pub. Date: Oct. 3, 2024

(54) CATHETERS INCLUDING METALLIC ALLOYS FOR INTRODUCTION OF THERAPEUTIC IONS

(71) Applicant: RAIN Scientific, Inc., Huntersville, NC (US)

(72) Inventors: TIM KOEHLER, Huntersville, NC (US); PAUL FRANKHOUSER, Huntersville, NC (US)

(21) Appl. No.: 18/286,418

(22) PCT Filed: Apr. 12, 2022
(86) PCT No.: PCT/US22/24434

§ 371 Date: Oct. 11, 2023

Related U.S. Application Data
(60) Provisional application No. 63/173,844, filed on Apr. 12, 2021.

(30) Foreign Application Priority Data
Apr. 12, 2021 (US) 63173844

(51) Int. Cl. A61M 25/00; A61N 1/04; A61N 1/30; A61N 1/32; C22C 5/08

(52) U.S. Cl. CPC A61M 25/0017; A61N 1/0436; A61N 1/044; A61N 1/306; A61N 1/325; C22C 5/08; A61M 2025/0019; A61M 2205/0205; A61M 2205/04

(57) ABSTRACT

The present invention is directed to devices and methods that efficiently and safely kill pathogens in blood functions by the generation of metal ions in situ. Such metal ions include silver ions, copper ions, and gold ions; however, additional metals can be employed for generation of ions in situ, including platinum, iridium, and zinc. The activity of the devices and methods of the present invention is extremely broad, and can kill pathogenic bacteria, viruses, and fungi. The activity of the devices and methods of the present invention does not depend on specific interactions between the device and the surface of the pathogen being killed. Additionally, the activity of the devices and methods is not subject to the development of resistance by the pathogen, such as can occur subsequent to the administration of conventional antibacterial, antifungal, or antiviral agents.

### CROSS-REFERENCE TO RELATED APPLICATIONS

This application claims the benefit of U.S. Provisional Application Ser. No. 63/173,844 by T. Koehler et al., entitled “Catheters Including Metallic Alloys for Introduction of Therapeutic Ions,” and filed on Apr. 12, 2021, the contents of which are incorporated herein by this reference.

### FIELD OF THE INVENTION

This invention is directed to devices and methods for killing pathogens in blood and treating diseases caused by the pathogens as well as devices and methods for stimulating the immune system, as well as devices and methods for delivering medicaments or fluids intravenously and devices and methods for draining bodily fluids.

### BACKGROUND OF THE INVENTION

Although great strides have been made in treating bacterial, viral, and fungal infections by the use of antibacterial, antiviral, and antifungal therapeutic agents, blood-borne pathogenic infections and airborne infections are still a matter of great concern in medical practice. Even infectious agents that primarily attack other tissues or organs, such as severe acute respiratory syndrome coronavirus 2, which causes COVID-19 and primarily attacks the lungs, can infect the blood which greatly complicates the treatment and control of COVID-19. More generally, pathogenic infections of the blood can have a devastating effect on a patient once the pathogen has infected its host. In many cases, such infections result in the death of the host. Among the most serious of blood-borne infections are such pathogens as: human immunodeficiency virus (HIV), the virus that causes AIDS; hepatitis A virus; hepatitis B virus; hepatitis C virus; Ebola virus; severe acute respiratory syndrome coronavirus 2; antibiotic-resistant bacteria such as MRSA (methicillin-resistant Staphylococcus aureus); and vector-borne viruses such as dengue virus, West Nile virus, and Zika virus. HIV can mutate rapidly and it can therefore be difficult to create an effective treatment for it; moreover, HIV is a retrovirus whose RNA genome can be reverse-transcribed into DNA, after which the DNA can be incorporated into the genome of cells that it infects. This can greatly complicate eradication of the virus; moreover, even after more than 20 years of research, no vaccine for the prevention of infection by HIV is yet available. For hepatitis C virus, there are many different strains of the virus and therefore available drug therapies such as antiviral agents can be ineffective and/or have debilitating side effects. The patients for whom the available antiviral agents are ineffective are classified as non-responders and their chances for survival are rather bleak. One particular strain of hepatitis C has been linked to a new form of liver cancer that was not previously seen. There has also been a rise in the occurrence of hepatitis C worldwide due to a number of factors, including a rise in the popularity of tattooing and the lack of proper hygiene associated with it, as well as the intravenous injection of street drugs such as heroin under unsafe conditions.

In particular, the treatment of viral infections has been complicated by the fact that viruses are structurally and biologically extremely diverse and have many different life cycles. Therefore, most antiviral drugs have a relatively limited spectrum of action as compared with the relatively broad spectra of action for many antibacterial antibiotics such as penicillin or azithromycin. For example, oseltamivir, an antiviral medication used to treat influenza, has essentially no activity against other viruses, including severe acute respiratory syndrome coronavirus 2. Moreover, even when used to treat seasonal influenza, oseltamivir must be administered early in the course of the disease for maximum effectiveness.

Other significant blood-borne bacterial pathogens include Staphylococcus epidermidis, Bacillus cereus, Enterococcus faecalis, Enterococcus faecium, Listeria monocytogenes, Streptococcus pneumoniae, Streptococcus pyogenes, Streptococcus agalactiae, Escherichia coli, Klebsiella pneumoniae, Haemophilus influenzae, Pseudomonas aeruginosa, Acetinobacter baumannii, Neisseria meningitidis, Bacteroides fragilis, Bacillus anthracis, Yersinia pestis, Francisella tularensis, and Brucella abortus (M. R. Pringle et al., “Multiplexed Identification of Blood-Borne Bacterial Pathogens by Use of a Novel 16S rRNA Gene PCR-Ligase Detection Reaction-Capillary Electrophoresis Assay,” J. Clin. Microbiol. 45: 1927-1935 (2007)).

Bacterial infections of particular significance are those caused by: methicillin-resistant Staphylococcus aureus, as stated above; Pseudomonas aeruginosa; and vancomycin-resistant Enterococcus species.

Blood-borne fungal infections include candidiasis, including infections caused by Candida auris; aspergillosis; blastomycosis; coccidioidomycosis; Cryptococcus neoformans infection; and Cryptococcus gattii infection.

Blood-borne protozoal infections include malaria.

Because of these drawbacks, attempts have been made to destroy blood-borne pathogens by use of metal ions.

One process that has been used to introduce metal ions for the treatment of blood-borne infectious diseases is iontophoresis. Iontophoresis is a physical process wherein ions are driven by an electrical field and flow diffusively through a medium. The prior art has used silver iontophoresis by inserting a catheter into the subclavian vein or the superior vena cava and then placing a silver probe through it and directly into the blood stream. A small electrical current is then applied to the wire in the prescribed amount to release the proper amount of silver nanoparticles, which have a slightly positive charge, to bond to viruses, which has a slightly negative charge. This process destroys the virus by disrupting the functions of the membrane of the virus and thus its ability to survive. This procedure has various challenges associated with it due to its close proximity to the heart, its duration of time needed to be successful and its chances of creating a secondary infection at the entry site.

The prior art also provides devices and methods by which blood is removed from the patient's body, re-routed through a device that uses nanoparticles to kill viruses, and the blood is then reinfused into the patient's body.

These devices and methods include the Biospleen device, developed at the Wyss Institute, which uses magnetic nanoparticles injected into the bloodstream of a patient; these nanoparticles have a protein attached to their surfaces which adheres to bacteria, viruses, and fungi. The Biospleen device then uses a magnet to pull out the nanoparticles and the pathogens attached to them. These devices and methods also include the Seraph 100 device, which uses microbeads coated with heparin to bind viruses and enable the device to remove the viruses from the bloodstream. These methods, however, are dependent upon large machines and the ability of a patient to remain “hooked up” to a machine for a long enough period of time such that an improvement in pathogen counts, such as viral counts, bacterial counts, or fungal counts, in the patient has been achieved.

The prior art also discloses a method and apparatus for destroying blood borne pathogens which utilizes a low-intensity direct current to generate positive particles from various metals which destroy viral pathogens (U.S. Pat. No. 6,539,252 to Fields et al.). A first electrode comprised of a metal such as silver is inserted into a patient's venous system. Then, a second electrode is placed on the patient's exterior in the vicinity of the first electrode. A low-intensity direct current is applied to the first metal electrode which releases silver cations that bond to the virus, resulting in the denaturing of the virus. The first electrode is placed in the venous system of the infected patient via a catheter. However, this prior art method relies upon using two separate electrodes where one is inserted into the patient's body and the other upon the skin of the patient, and, depending on the patient, may further require an electrode to be inserted into the patient's jugular vein. This prior art arrangement is complex and may result in complications such as infection due to the multiple insertions required.

U.S. Pat. No. 9,849,282 to Fuller et al. discloses a medical implant device for inhibiting infection associated with a joint prosthesis implant employing a current. However, as the medical implant device is completely internally implanted, the options for control and programming of the device are extremely limited.

Other alternatives for use of electrical current to treat pathogens are described in United States Patent Application Publication No. 2020/0009375 by Koehler.

New approaches are required for treatment of blood-borne infections employing the antimicrobial activity of metal ions because of the less than optimal performance of prior art devices and methods.

Current alternatives for antimicrobial catheters employ a coating that relies on a chemical reaction for release of an antimicrobial agent. Typically, the released antimicrobial agent is an ion, most commonly a silver ion. This chemical release is uncontrolled, not consistent in its operation, and diminishes over time. Again, new approaches are required for treatment of blood-borne infections employing the antimicrobial activity of metal ions because of the less than optimal performance of prior art devices and methods.

Thus, there exists a long-felt need for a device and method that efficiently and safely kills pathogens in blood without the need to remove the blood from the patient to achieve the desired result with a minimally invasive technique and a minimum of risk of complications such as infections. Preferably, such a device and method can kill a broad range of pathogens, including viruses, bacteria, fungi, and protozoa, and does not depend for its activity on specific interactions between the device and the surface of the pathogen. Preferably, such a device is not subject to the development of resistance to the device by the pathogens being treated, such as can occur with conventional antibacterial, antiviral, antifungal, and antiprotozoal pharmaceutical agents.

Furthermore, there exists a need for improved antimicrobial catheters with a consistent effect that does not diminish over time.

### SUMMARY OF THE INVENTION

The present invention is directed to a device and method that efficiently and safely kills pathogens in blood functions by the generation of metal ions in situ. Such metal ions include silver ions, copper ions, and gold ions; however, additional metals can be employed for generation of ions in situ, including platinum, iridium, and zinc. The activity of the device and method of the present invention is extremely broad, and can kill pathogenic bacteria, viruses, fungi, and protozoa. The activity of the device and method of the present invention does not depend on specific interactions between the device and the surface of the pathogen being killed. Additionally, the activity of the device and method is not subject to the development of resistance by the pathogen, such as can occur subsequent to the administration of conventional antibacterial, antifungal, antiviral, or antiprotozoal agents.

One aspect of the present invention is a device for treatment or prevention of a bacterial, viral, fungal, or protozoan infection or the stimulation of the immune system comprising:
(a) a wire comprising a metal or metallic alloy that releases ions when a low-intensity direct current is applied to the wire;
(b) an insulated covering insulating the wire, wherein the wire and the insulated covering are inserted into the bloodstream of a patient to be treated via a catheter;
(c) a male connector;
(d) a female connector in operable contact with the male connector, such that the male connector and the female connector protect components of the device and enable connection to the catheter;
(e) a pin of conductive material within the male connector to transmit a low-intensity direct current from a power supply connected to the device; and
(f) optionally, a side port for introduction of a medication.

In another version of this alternative of the device, the catheter can include a cable that provides a direct connector to the wire so that the cable is actually part of the catheter.

In yet another version of this alternative of the device, the pin of conductive material, typically copper, can be replaced with a heat shrink connector that incorporates a circular solder ring. For the use of the heat shrink connector, two wires that are to be connected are placed in opposite ends of the connector so that the wires adjoin within the circular solder ring, heat is applied to the external connector surface which activates the solder ring to connect the wires, followed by the exterior plastic material shrinking down and encapsulating the connected wires. This alternative can be employed in other embodiments of the present invention described as having a copper conductive pin or a conductive pin of another conductive metal.

The device can further comprise a power supply in electrical contact with the device. In one alternative, the power supply in electrical contact with the device has the ability to adjust the current based on the therapeutic application of the device.

In this and similar devices as described below, the wire comprising a metal or metallic alloy is typically selected from the group consisting of:
(1) an alloy of gold and silver in which the alloy comprises 70% or more of gold and 30% or less of silver;
(2) an alloy of gold and silver in which the alloy comprises 70% or more of silver and 30% or less of gold;
(3) an alloy of gold, silver, and copper in which the alloy comprises 70% or more of gold and a total of 30% or less of silver and copper;
(4) an alloy of silver, copper, and gold in which the alloy comprises 70% or more of silver and a total of 30% or less of copper and gold;
(5) an alloy of silver, copper, and gold in which the alloy comprises 70% or more of copper and a total of 30% or less of silver and gold;
(6) an alloy of silver, copper, and gold in which the alloy comprises between 1% and 99% each of gold, silver, and copper;
(7) an alloy of gold and one or more of silver, copper, platinum, iridium, and zinc in which the alloy comprises 70% or more of gold, and a total of 30% or less of silver, copper, platinum, iridium, or zinc;
(8) an alloy of silver and one or more of gold, copper, platinum, iridium, and zinc in which the alloy comprises 70% or more of silver and a total of 30% or less of gold, copper, platinum, iridium, or zinc;
(9) an alloy of copper and one or more of gold, silver, platinum, iridium, and zinc in which the alloy comprises 70% or more of copper and a total of 30% or less of gold, silver, platinum, iridium, or zinc;
(10) an alloy of platinum and one or more of gold, silver, copper, iridium, and zinc in which the alloy comprises 70% or more of platinum and a total of 30% or less of gold, silver, copper, iridium, or zinc;
(11) an alloy of iridium and one or more of gold, silver, copper, platinum, and zinc in which the alloy comprises 70% or more of iridium and a total of 30% or less of gold, silver, copper, platinum, or zinc; and
(12) an alloy of zinc and one or more of gold, silver, copper, platinum, and iridium in which the alloy comprises 70% or more of zinc and a total of 30% or less of gold, silver, copper, platinum, or iridium. However, other alternatives for these wires are within the scope of the present invention.

Typically, the device releases from about 2×10⁹ to about 7×10¹² ions per second in operation. Typically, the device employs a voltage of about 1.2 V or less. Preferably, the device employs a voltage of from about 0.5 V to about 0.92 V; in some alternatives, a voltage of 0.87 V or 0.89 V is employed. Typically, the device employs a current of less than about 10 μA. Preferably, the device employs a current of less than about 6 μA. More preferably, the device employs a current of about 4.9 μA.

In one alternative, the device provides constant current with a cap on voltage. In another alternative, the device provides constant voltage with a cap on current.

Another aspect of the present invention is a device for treatment or prevention of a bacterial, viral, fungal, or protozoan infection or the stimulation of the immune system comprising:
(1) a wire comprising a metal or metallic alloy that releases ions when a low-intensity direct current is applied to the wire;
(2) an insulated covering insulating the wire, wherein the wire and the insulated covering are inserted into the bloodstream of a patient to be treated via a catheter;
(3) a male connector;
(4) a female connector in operable contact with the male connector, such that the male connector and the female connector protect components of the device and enable connection to the catheter;
(5) a pin of conductive material within the male connector to transmit a low-intensity direct current from a power supply connected to the device;
(6) a stylet for insertion into a vein of a subject to be treated;
(7) insulation to insulate the stylet; and
(8) optionally, a side port for introduction of a medication.

In another version of this alternative of the device, the stylet can include a cable that provides a direct connector to the wire so that the cable is actually part of the stylet.

In this alternative, the wire comprising the metal or metallic alloy is as described above. The rate of release of ions, the voltage, and the current are as described above.

This alternative for the device can further comprise a power supply as described above.

Yet another aspect of the present invention is a device for treatment or prevention of a bacterial, viral, fungal, or protozoan infection or the stimulation of the immune system comprising:
(a) a wire comprising a metal or metallic alloy that releases ions when a low-intensity direct current is applied to the wire;
(b) an insulated covering that insulates a portion of the wire;
(c) a connector; and
(d) an electrode that rests on top of the skin of a patient when the insulated covering and the wire are inserted into the vein of a patient.

In this alternative, the wire comprising the metal or metallic alloy is as described above. The rate of release of ions, the voltage, and the current are as described above. In this alternative, the electrode that rests on top of the skin of the patient is typically a conductive patch such as an EKG patch that, connected to a cable connecting to the power supply, completes the electric circuit. This electrode acts as the cathode.

This alternative for the device can further comprise a power supply as described above.

Still another aspect of the present invention is a device for treatment or prevention of a bacterial, viral, fungal, or protozoan infection or the stimulation of the immune system comprising:
(1) a wire comprising a metal or metallic alloy that releases ions when a low-intensity direct current is applied to the wire;
(2) an insulating covering that insulates a portion of the wire;
(3) a domed cap covering one end of the wire;
(4) one or more cut-outs that expose a portion of the wire for delivery of the ions when the low-intensity direct current is applied to the wire;
(5) a male connector;
(6) a female connector in operable contact with the male connector, such that the male connector and the female connector protect components of the device and enable connection to a catheter or stylet for insertion of the device; and
(7) optionally, a side port for introduction of a medication.

In another alternative of this embodiment of the device, the catheter or stylet can include a cable that provides a direct connection to the wire so that the cable is actually part of the catheter. Alternatively, one of the female connector or the male connector could include a pin of conductive material as described above.

In this aspect of the invention, the length of the insulated wire extending from the proximal or originating point of the male connector can range from:
(a) about 1.5 inches (3.81 cm) to about 6 inches (15.24 cm) for use with a peripheral venous electrode;
(b) about 4 inches (10.16 cm) to about 9 inches (22.86 cm) for a central venous electrode; or
(c) about 6 inches (15.24 cm) to about 32 inches (81.28 cm) for an electrode placed within a peripherally insulated central catheter (PICC).

In this alternative, the wire comprising the metal or metallic alloy is as described above. The rate of release of ions, the voltage, and the current are as described above.

This alternative for the device can further comprise a power supply as described above.

Yet another aspect of the present invention is a device for treatment or prevention of a bacterial, viral, fungal, or protozoan infection or the stimulation of the immune system comprising:
(1) a wire comprising a metal or metallic alloy that releases ions when a low-intensity direct current is applied to the wire, wherein the wire is coiled or braided around a catheter for insertion of the device into the bloodstream of a patient; and
(2) two cap connectors in electrical contact with the wire, wherein each cap connector is connected to a power supply connected to the device to transmit a low-intensity direct current to the device; and
(3) a power supply connected to the two cap connectors.

In this alternative, the wire can be arranged relative to the catheter in an alternative selected from the group consisting of:
(1) spirally externally to the catheter;
(2) spirally internally to the catheter;
(3) hexagonally externally to the catheter;
(4) hexagonally internally to the catheter;
(5) braided externally to the catheter; and
(6) braided internally to the catheter.

In this alternative, the wire comprising the metal or metallic alloy is as described above. The rate of release of ions, the voltage, and the current are as described above.

This alternative for the device can further comprise a power supply as described above.

Still another aspect of the present invention is a device for treatment or prevention of a bacterial, viral, fungal, or protozoan infection or the stimulation of the immune system comprising:
(1) a wire comprising a metal or metallic alloy that releases ions when a low-intensity direct current is applied to the wire;
(2) an insulated covering that covers the entire wire except for one or more cut-outs;
(3) a domed cap that seals the open end of the insulated covering; and
(4) a power supply that is connected to the device to transmit the low-intensity direct current to the device.

In this alternative, the domed cap covers the distal end of a stylet or catheter used to insert the wire into the vein, so that no bare wire is exposed to cause potential irritation or to damage the internal venous wall. In another alternative of this embodiment of the device, the device can include a cable that provides a direct connector to the wire so that the cable is actually part of the stylet. In yet another alternative of this device, the device can include a pin of conductive material as described above.

In this alternative, the wire comprising the metal or metallic alloy is as described above. The rate of release of ions, the voltage, and the current are as described above.

Yet another aspect of the present invention is a device for treatment or prevention of a bacterial, viral, fungal, or protozoan infection or the stimulation of the immune system comprising:
(1) a wire comprising a metal or metallic alloy that releases ions when a low-intensity direct current is applied to the wire, the wire being inserted into the vein of a patient via a catheter;
(2) an insulated covering to protect the wire;
(3) a cap connector;
(4) a male connector attached near one end of the wire;
(5) a female connector attached closer to the end of the wire to which the male connector is attached than the male connector;
(6) a pin of conductive material within the male connector to transmit a low-intensity direct current from a power supply connected to the device; and
(7) optionally, a side port for introduction of a medication.

In this alternative, the wire comprising the metal or metallic alloy is as described above. The rate of release of ions, the voltage, and the current are as described above.

In yet another version of this alternative of the device, the pin of conductive material, typically copper, can be replaced with a heat shrink connector that incorporates a circular solder ring as described above.

This alternative for the device can further comprise a power supply as described above.

Still another aspect of the present invention is a device for treatment or prevention of a bacterial, viral, fungal, or protozoan infection or the stimulation of the immune system comprising:
(1) a wire within a stylet comprising a metal or metallic alloy that releases ions when a low-intensity direct current is applied to the wire, the wire being inserted into the vein of a patient by insertion of the stylet;
(2) an insulated covering surrounding the wire;
(3) a domed cap, wherein the insulated covering extends all the way to the domed cap;
(4) one or more cut-outs providing access for the wire to the bloodstream of a patient;
(5) a male connector;
(6) a female connector;
(7) a pin of conductive material within the male connector to transmit a low-intensity direct current from a power supply connected to the device, the pin being protected by the male connector and the female connector; and
(8) optionally, a side port for introduction of a medication;
   wherein the device is adapted for use with a PICC catheter (peripherally insulated central catheter).

In this alternative, the wire comprising the metal or metallic alloy is as described above. The rate of release of ions, the voltage, and the current are as described above.

In yet another version of this alternative of the device, the pin of conductive material, typically copper, can be replaced with a heat shrink connector that incorporates a circular solder ring as described above.

This alternative for the device can further comprise a power supply as described above.

Yet another aspect of the present invention is a device for treatment or prevention of a bacterial, viral, fungal, or protozoan infection or the stimulation of the immune system comprising:
(1) an open lumen catheter;
(2) an hemostatic seal attached to the catheter to close off space around the catheter;
(3) a side port to allow the administration of an intravenous antibiotic or other medication; and
(4) a wire comprising a metal or metallic alloy that releases ions when a low-intensity direct current is applied to the wire, the wire being inserted into the vein of a patient via the catheter, wherein the inner diameter of the catheter is greater than the outer diameter of the wire to allow for both the insertion of the wire and as an auxiliary venous medication delivery port via the side port and in combination with a proximal hemostatic seal.

In this alternative, the wire comprising the metal or metallic alloy is as described above. The rate of release of ions, the voltage, and the current are as described above.

This alternative for the device can further comprise a power supply as described above.

Still another aspect of the present invention is a device for treatment or prevention of a bacterial, viral, fungal, or protozoan infection or the stimulation of the immune system comprising:
(1) a catheter;
(2) wiring surrounding the catheter, the configuration of the wiring selected from the group consisting of:
(a) external spiral-wound wiring surrounding the catheter;
(b) straight longitudinal wiring surrounding the catheter;
(c) wiring in the shape of rings at the distal end of the catheter, the proximal end of the catheter, or both the distal end and the proximal end of the catheter;
(d) wiring in a hexagonal pattern external to the catheter; and
(e) wiring with periodically exposed sections of the wire from the proximal end to the distal end of the catheter, or, alternatively, at the distal end of the catheter only;
   wherein the wiring comprises a metal or metallic alloy that releases ions when a low-intensity direct current is applied to the wire; and
(3) a connector connecting the wiring in the hex design to a power supply to transmit a low-intensity direct current to the device.

In this alternative, the wire comprising the metal or metallic alloy is as described above. The rate of release of ions, the voltage, and the current are as described above.

This alternative for the device can further comprise a power supply as described above.

Yet another aspect of the present invention is a device for treatment or prevention of a bacterial, viral, fungal, or protozoan infection or the stimulation of the immune system comprising:
(1) a catheter with either a single lumen or multiple lumens;
(2) wiring implanted into the catheter with skived windows implanted into the catheter, wherein the wiring comprises a wire of metal or metallic alloy that releases ions when a low-intensity direct current is applied to the wire; and
(3) a connector connecting the wiring to a power supply to transmit a low-intensity direct current to the device.

In this alternative, the wire comprising the metal or metallic alloy is as described above. The rate of release of ions, the voltage, and the current are as described above.

This alternative for the device can further comprise a power supply as described above.

Still another aspect of the present invention is a device for treatment or prevention of a bacterial, viral, fungal, or protozoan infection or the stimulation of the immune system comprising a wound care bandage or patch, wherein the wound care bandage or patch comprises:
(1) a substantially planar patch or bandage having a top side and a bottom side;
(2) a coating on the bottom side of the patch or bandage, the coating including therein a first wire that comprises a metal or metallic alloy that releases ions when a low-intensity direct current is applied to the first wire;
(3) a snap connector on the top side of the patch or bandage;
(4) a second wire in electrical contact with the snap connector; and
(5) a power supply to transmit a low-intensity direct current to the device, wherein the power supply is in electrical contact with the first and second wires.

In this alternative, the wire comprising the metal or metallic alloy is as described above. The rate of release of ions, the voltage, and the current are as described above.

Yet another aspect of the present invention is a device for treatment or prevention of a bacterial, viral, fungal, or protozoan infection or the stimulation of the immune system for incorporation into an intravenous line or other device intended to supply a fluid, the device comprising:
(1) a hubcap that acts as a cover for the device;
(2) a cell battery acting as a power source;
(3) a printed circuit board in electrical contact with the cell battery to control the current output;
(4) a cathode wire comprising a metal or metallic alloy;
(5) an anode wire comprising a metal or metallic alloy;
(6) a fluid-absorbing material that is in electrical contact with the cathode wire and the anode wire to provide an electrical circuit when fluid flows through the device;
(7) a first connector;
(8) a second connector, wherein the first connector and the second connector enable placement of the device into an intravenous line to supply fluid to a patient;
(9) an anode metallic pin joining the first connector and the second connector; and
(10) a cathode metallic pin joining the first connector and the second connector; wherein the device is adapted for insertion into an intravenous line or other device intended to supply a fluid.

In this alternative, the cathode wire and anode wire comprising the metal or metallic alloy are as described above. The rate of release of ions, the voltage, and the current are as described above.

Still another aspect of the present invention is a device for treatment or prevention of a bacterial, viral, fungal, or protozoan infection or the stimulation of the immune system adapted for attachment to a fluid source comprising:
(1) a hubcap that acts as a cover for the device;
(2) a cell battery acting as a power source;
(3) a printed circuit board in electrical contact with the cell battery to control the current output;
(4) a cathode wire comprising a metal or metallic alloy;
(5) an anode wire comprising a metal or metallic alloy;
(6) a fluid-absorbing material that is in electrical contact with the cathode wire and the anode wire to provide an electrical circuit when fluid flows through the device;
(7) a solid cap;
(8) a connector;
(9) a fluid source in operable contact with the connector;
(10) an anode metallic pin in operable contact with the connector; and
(11) a cathode metallic pin in operable contact with the connector.

In this alternative, the cathode wire and anode wire comprising the metal or metallic alloy are as described above. The rate of release of ions, the voltage, and the current are as described above.

Yet another aspect of the present invention is a device for treatment or prevention of a bacterial, viral, fungal, or protozoan infection or the stimulation of the immune system comprising:
(1) a generator attached to the skin of a patient;
(2) means for regulating the output of the generator; and
(3) an implanted stylet comprising a wire, wherein the generator is in electrical contact with the wire to transmit a low-intensity direct current through the wire and into the skin of the patient.

In this alternative, the cathode wire and anode wire comprising the metal or metallic alloy are as described above. The rate of release of ions, the voltage, and the current are as described above.

Still another aspect of the present invention is a device comprising a wafer or layer of a chewable gum that has particles of a metal or metallic alloy incorporated therein which are released on chewing with the hydrolytic action of saliva enabling the release of the particles so that they are absorbed into the bloodstream. The metal or metallic alloy is as described above. Such devices according to the present invention can be adjunctive to the infusion therapy described herein.

Yet another aspect of the present invention is a device for treatment or prevention of a bacterial, viral, fungal, or protozoan infection or the stimulation of the immune system comprising:
(1) a catheter;
(2) a wire internally run within the catheter;
(3) one to five external conductive rings surrounding the catheter;
(4) an insulated wire comprising a metal or metallic alloy;
(5) a connector in operable contact with the catheter;
(6) a wire placed inside the connector for connection to a power supply to transmit a low-intensity direct current to the device, wherein the power supply is in electrical contact with the wire; and
(7) optionally, a side port for introduction of a medication.

In this alternative, the wire comprising the metal or metallic alloy is as described above. The rate of release of ions, the voltage, and the current are as described above.

This alternative for the device can further comprise a power supply as described above.

Yet another aspect of the present invention is a device for treatment or prevention of a bacterial, viral, fungal, or protozoan infection or the stimulation of the immune system comprising:
(1) an introducer needle;
(2) a dual-lumen or multi-lumen catheter;
(3) a primary lumen for fluid flow within the dual-lumen or multi-lumen catheter;
(4) a secondary lumen within the dual-lumen or multi-lumen catheter;
(5) an insulated stylet for insertion into the secondary lumen of the dual-lumen or multi-lumen catheter;
(6) a wire comprised of a metal or metallic alloy in electrical contact with the insulated stylet;
(7) a removable spacer;
(8) a connector adjacent to the removable spacer to attach the insulated stylet to the dual-lumen or multi-lumen catheter;
(9) an internal copper pin for attachment to a power supply;
(10) an injection side port for adding a fluid; and
(11) a hemostatic seal to prevent the backflow of blood from the secondary lumen where stylet is placed.

In this alternative, the wire comprising the metal or metallic alloy is as described above. The rate of release of ions, the voltage, and the current are as described above.

In yet another version of this alternative of the device, the copper pin of conductive material can be replaced with a heat shrink connector that incorporates a circular solder ring.

This alternative for the device can further comprise a power supply as described above.

In this alternative, the device can be used with a catheter selected from the group consisting of a central catheter, a mid-line catheter, a PICC catheter, and a central venous catheter.

Still another aspect of the present invention is a needleless injector stylet comprising:
(1) a proximal end for connection to a fluid-containing device; and
(2) a distal end for insertion into a vein.

Yet another aspect of the present invention is a device for treatment or prevention of a bacterial, viral, fungal, or protozoan infection, stimulation of the immune system, or providing a zone of inhibition around a catheter placement to protect against the development of microbial biofilm or infection from the puncture site comprising an extracorporeal catheter comprising:
(1) a catheter for insertion into a vein, the catheter having a proximal end and a distal end;
(2) a battery-driven power supply;
(3) a circuit board in operable contact with the battery-driven power supply for the control of voltage and current supplied by the device;
(4) a cathode;
(5) an anticoagulant coating covering the cathode;
(6) ion-producing anode wires, which comprise from one to five wires comprising a metal or metallic alloy; and
(7) a pump to promote blood flow.

In this alternative, the wires comprising the metal or metallic alloy are as described above. The rate of release of ions, the voltage, and the current are as described above.

Yet another aspect of the present invention is a method of treating a blood-borne pathogenic infection, such as a bacterial, viral, fungal, or protozoan infection, stimulating the immune system, or providing a zone of inhibition around a catheter placement to protect against the development of microbial biofilm or infection from the puncture site, comprising the steps of:
(1) placing a device for treatment or prevention of a bacterial, viral, fungal, or protozoan infection, stimulation of the immune system, or providing a zone of inhibition around a catheter placement as described above into operable contact with a subject in need of treatment of a blood-borne pathogenic infection such that the device can provide metallic ions to the bloodstream of the subject; and
(2) causing the device to release metallic ions to the bloodstream of the subject to treat the infection, stimulate the immune system, or provide a zone of inhibition around a catheter placement. The current market for antimicrobial catheters involves utilization of some sort of coating that relies on a chemical reaction for antimicrobial release; in most cases the chemical reaction releases ions, most commonly silver ions. The use of such chemical reactions is uncontrolled, inconsistent, and results in diminishing release of the antimicrobial ions over time. In contrast, devices and methods according to the present invention utilize a current-driven system, so that the release of antimicrobial ions is controlled, consistent, and sustained over the life of the placement of the device according to the present invention. The antimicrobial components of devices according to the present invention are replaceable so that the device can provide superior protection for the desired life of the catheter insertion. Additionally, in some embodiments of a device according to the present invention, a stylet can be swapped out without removing the entire catheter, in contrast to prior art devices in which the entire catheter needs to be removed and another catheter inserted, which increases the risk of infection or other complications.

Typically, the device is placed into operable contact with the subject by placing a wire of the device into a vein of the subject so that the wire releases metallic ions into the bloodstream of the patient. The device can be placed in-line into an intravenous line, or placed in-line into a fluid source.

When the method is used to treat a bacterial infection, typically, the bacterial infection is an infection caused by a bacterium selected from the group consisting of Staphylococcus aureus, Staphylococcus epidermidis, Bacillus cereus, Enterococcus faecalis, Enterococcus faecium, Listeria monocytogenes, Streptococcus pneumoniae, Streptococcus pyogenes, Streptococcus agalactiae, Escherichia coli, Klebsiella pneumoniae, Haemophilus influenzae, Pseudomonas aeruginosa, Acetinobacter baumannii, Neisseria meningitidis, Bacteroides fragilis, Bacillus anthracis, Yersinia pestis, Francisella tularensis, and Brucella abortus. In particular, the method can be used to treat an infection caused by Staphylococcus aureus, wherein the Staphylococcus aureus is methicillin-resistant Staphylococcus aureus (MRSA).

Devices and methods according to the present invention can also be employed as: a stand-alone treatment for blood-borne pathogens, including bacteria, viruses, fungi, and protozoa; means to modulate the immune system; and means to provide a zone of inhibition around a catheter placement to fight against microbial biofilm and/or infection from the puncture site of the catheter.

When the method is used to treat a viral infection, typically the viral infection is an infection caused by a virus selected from the group consisting of human immunodeficiency virus (HIV), hepatitis A virus, hepatitis B virus, hepatitis C virus, Ebola virus, Zika virus, dengue virus, West Nile virus, dengue virus, and severe acute respiratory syndrome coronavirus 2.

When the method is used to treat a fungal infection, typically the fungal infection is an infection selected from the group consisting of candidiasis, aspergillosis, blastomycosis, coccidioidomycosis, infection by Cryptococcus neoformans, and infection by Cryptococcus gattii.

When the method is used to treat a protozoan infection, typically the protozoan infection is malaria; however, other protozoan infections can also be treated.

The method can further comprise administration of a therapeutically effective quantity of an antimicrobial agent, which can be selected from the group consisting of an antibacterial agent, an antiviral agent, an antifungal agent, and an antiprotozoan agent.

The method can further comprise the steps of:
(3) subsequent to the initiation of administration of treatment, determining the results of the treatment by detecting or determining the quantity or concentration of the blood-borne pathogen remaining in the blood of the subject; and
(4) if necessary, adjusting the treatment by altering the quantity of ions administered, the current, the voltage, or the waveform used, or, if an additional antibacterial, antiviral, or antifungal agent is administered, altering a factor selected from the group consisting of the identity of the additional agent being administered, the dosage of the additional agent, the frequency of administration of the additional agent, the duration of administration of the additional agent, the route of administration of the additional agent, or another factor associated with the administration of the additional agent.

Still another aspect of the present invention is a method of stimulating the production of natural killer (NK) cells comprising the steps of:
(a) placing a device for treatment or prevention of a bacterial, viral, fungal, or protozoan infection or the stimulation of the immune system as described above into operable contact with a subject in need of stimulating the production of natural killer cells such that the device can provide metallic ions to the bloodstream of the subject; and
(b) causing the device to release metallic ions to the bloodstream of the subject to stimulate the production of natural killer cells and other natural immune responses of the body to treat neoplasms, including breast cancer.

In methods according to the present invention, means to stimulate the immune system can involve the electric field created by the generator and transmitted via the catheter or stylet activating calcium influx and efflux channels on lymphocytes, which, in turn, activates signaling in the immune system.

Methods according to the present invention can also be used for immune system stimulation.

### BRIEF DESCRIPTION OF THE DRAWINGS

These and other features, aspects, and advantages of the present invention will become better understood with reference to the following description, appended claims, and accompanying drawings where:

FIG. 1 is a front view of a device for killing pathogens in the blood of a patient according to selected embodiments of the present invention.

FIG. 2 is another front view of a device for killing pathogens in the blood of a patient according to selected embodiments of the present invention.

FIG. 3 is a front view and a side view of a device for killing pathogens in the blood of a patient according to selected embodiments of the present invention.

FIG. 4 is a series of front views of a device for killing pathogens in the blood of a patient according to selected embodiments of the present invention.

FIG. 5 is another series of front views of a device for killing pathogens in the blood of a patient according to selected embodiments of the present invention.

FIG. 6 is a series of front perspective views of a device for killing pathogens in the blood of a patient according to selected embodiments of the present invention.

FIG. 7 is a series of front views of the device mounted with a catheter inserted into the vein or artery of a patient.

FIG. 8 is a series of front views of a PICC device.

FIG. 9 is a series of front views of an open lumen catheter for dual therapy. In this figure, an hemostatic seal has been attached to close off around the catheter, which in this case is a specifically designed catheter.

FIG. 10 is a diagram of several catheter designs for a “zone of inhibition” that deliver positively charged ions to stimulate the immune system and to kill pathogens in the blood: FIG. 10A has spiral-wound wiring, either internal or external; FIG. 10B has straight longitudinal wiring, with three to five wires that are either internally exposed, externally exposed, or both internally and externally exposed; FIG. 10C has rings at the distal end, the proximal end, or both; all rings can be anode, or anodes and cathodes can alternate every other ring, or the catheter can be used with an external catheter cathode, such as used in an EKG path; FIG. 10D has a hex design, distal, proximal, or both, with a proximal external surface, and a distal internal or external surface, or with a proximal internal surface and a distal internal or external surface, with a cathode externally applied, such as used in an EKG path; FIG. 10E has periodically exposed sections of wire from the proximal end to the distal end, or at the distal end only, with windows from 1 mm to 10 mm in length and from 35°-180° exposure; and FIG. 10F shows end views of the arrangements (i) with 3 30° windows; and (ii) with 1 180° window.

FIG. 11 shows alternatives for designs of a catheter according to the present invention using either a single- or multi-lumen design that could be either intended for peripheral, central, midline and central, or peripherally inserted venous catheters (PICC) insertion: FIG. 11A shows: (i) a side view of a dual lumen with metallic alloy implanted into the catheter with skived windows at the distal end for ionic release to: (A) create a zone of inhibition (antimicrobial activity); and (B) release positively charged ions (cations) to stimulate the immune system utilizing an external cathode such as used in an EKG path; and (ii) an end view of the single lumen and electrode; FIG. 11B is an end view of the catheter of FIG. 11A; and FIG. 11C shows an end view of a similar multi-lumen catheter.

FIG. 12 is a schematic diagram of a wound care or burn care bandage according to the present invention. FIG. 12A shows a patch of varying size for application to an area of skin that incorporates into the dermal side of the patch (the side that comes into contact with the skin), an ion-releasing metallic alloy as described further below. The ion-releasing metallic alloy receives current when connected to a low-intensity direct current generator as described further below. FIG. 12B shows the lower side of the wound care or burn care bandage, with the incorporated ion-releasing metallic alloy showing the cathode and anode.

FIG. 13 is a diagram of a device according to the present invention that can be connected to other devices such as a catheter, an intravenous line, or another device through Luer locks, IV hose clamps, IV hose attachments, or other conventional fittings for connecting medical fluid flow components. In FIG. 13, X and Y show a pair of connectors for connection of the device to other devices, such as an intravenous line, with fluid flow through the device. The device has anode and cathode metallic pins which go to opposite sides of the device and a hubcap on the top surface of the device. The device contains a sintered porous plastic sheet (Porex®); the cathode is surrounded by the sintered porous plastic sheet to absorb fluid and to allow the completion of the circuit, anode to cathode. The device also contains a small printed circuit board to control current output. When fluid passes through the connector and through the sintered porous plastic sheet, a complete circuit is formed and ions are released. The anode and cathode are constructed ion-releasing metallic alloys as described above. In one alternative, one of the connections is made to a fluid source such as a water bottle or container for intravenous fluid.

FIG. 14 is a diagram of an alternative embodiment of a device according to the present invention intended for insertion into a vein, typically in a limb such as an arm. In this alternative embodiment, a generator producing a low-intensity direct current is attached to an ion-releasing alloy as described above. A temporarily implanted stylet or catheter is inserted into the vein. This alternative embodiment of a device according to the present invention can also be inserted into an intravenous line to release ions into the line. In this application, the device is attached to a small container of distilled water or other fluid suitable for administration via an intravenous line into which ions can be released; when turned upside down, the fluid activates the release of ions for a desired period, creating an ion-filled solution.

FIG. 15 is a diagram of another alternative embodiment of a device according to the present invention that is intended to transmit metallic ions as described above into the vein of a patient via a temporarily implanted stylet or catheter.

FIG. 16 is a diagram of another alternative embodiment of a device according to the present invention that is a chewable gum that continuously releases metallic ions into the bloodstream. Absorption can, alternatively occur orally.

FIG. 17 is a diagram of yet another alternative embodiment of a device according to the present invention that is a cathode catheter.

FIG. 18 is a diagram of a needleless injector stylet according to the present invention.

FIG. 19 is a diagram of yet another alternative embodiment of a device according to the present invention that incorporates a dual lumen catheter for fluid delivery, an injection side port, and a hemostasis valve or hemostatic seal to prevent the backflow of blood from the secondary lumen of the catheter.

FIG. 20 is a diagram of an extracorporeal catheter according to the present invention that includes a battery as a power supply, a circuit board to regulate the voltage and amperage, cathode and anode wiring with the cathode covered by an anticoagulant coating, and a pump to promote blood flow. The extracorporeal catheter is designed to be inserted into larger veins for the purposes of immune modulation and treatment of blood-borne pathogens.

FIG. 21 is a chart showing the results of treatment of various diseases by devices according to the present invention is shown in FIG. 21. The diseases treatable by devices according to the present invention as shown in FIG. 21 include HIV, dengue fever, infection with HSV-1, and infection with HSV-2. The results in FIG. 21 include the results of detailed blood work.

### DETAILED DESCRIPTION OF THE INVENTION

The present invention employs the use of ions generated by an ion-producing metal by passage of a low-intensity direct current through the ion-producing metal. As used herein, the term “ion-producing metal” refers to: (i) a metal or metalloid element selected from the group consisting of silver, copper, gold, platinum, iridium, zinc, palladium, and osmium; and (ii) an alloy of two or more metal or metalloid elements, each of which is selected from the group consisting of silver, copper, gold, platinum, iridium, zinc, palladium, and osmium. As detailed below, the ion-producing metal is formed into an electrode for insertion into the patient; typically, as described below, the electrode is inserted into the venous system, such as through a catheter. As used herein, the term “catheter” refers to a conventional catheter, typically Teflon, polyurethane, silastic, or some other form of plastic or other biologically compatible materials, of a length and diameter consistent with the devices described herein. Catheters of a length and diameter consistent with the devices described herein and constructed of materials described herein can also be used to drain body fluids to protect the patient from nosocomial infections; such catheters include urinary catheters.

Although Applicants do not intend to be bound by this theory, it is believed that silver ions, in particular, can attach to the membranes of immune cells and depolarize them from about −70 mV to about −50 mV, where a current of positively charged sodium and calcium ions flow back into the cells through pores in the membrane called ion channels. This constitutes a signal that results in altered transcription of genes that lead to the cells becoming activated. Therefore, a current creates metal ions, such as, but not limited to, silver ions in the bloodstream which, in turn, lead to the immune cells becoming activated.

Additionally, devices and methods according to the present invention use a current that is substantially less than in devices such as pacemakers, neurostimulators, or TENS units. One aspect of the function of devices and methods according to the present invention is to facilitate antigen presentation along with current-induced calcium ion influx in lymphocytes which, in return, activates lymphocyte activation and proliferation. Moreover, virus-infected cells are more vulnerable to current than are healthy eukaryotic cells. It is well known that excess calcium ion influx and depolarization are hallmarks of many viral infections. For this reason, although Applicants do not intend to be bound by this theory, current from devices and methods according to the present invention would have a greater impact on viral-infected cells as compared with non-infected eukaryotic cells.

In another aspect of the invention, the penetration of ions produced by devices and methods according to the present invention in the outer membrane of bacteria is enhanced by porins in the outer membrane. This increases the ability of the ions to prevent the replication of the bacteria. Additionally, the penetration of the ions into the bacteria can induce denaturation of the bacteria, which facilitates antigen production and the subsequent presentation of antigens to lymphocytes.

Although Applicants do not intend to be bound by this theory, devices and methods according to the present invention are hypothesized to modulate the immune system by inducing calcium influx in lymphocytes which in return signals the immune system. When antigens bind to a lymphocyte, potassium channels open and the efflux of K⁺ ions out of the cell create a hyperpolarization that makes the cell even more negatively charged. This causes voltage-sensitive Ca²⁺ channels to open to allow an influx of Ca²⁺ ions that creates a signal inside the lymphocyte. This signal causes the lymphocyte to do the following things: (i) change the shape or volume of the cell; (ii) activate certain enzymes in the activation cascade; and (iii) turn on genes that express K⁺ and Ca²⁺ ion channels. In addition, the Ca²⁺ influx has also been shown to activate both T cells and NK cells which play a significant role in fighting infections and cancer within the body. The more K⁺ and Ca²⁺ channels present in a cell membrane, the greater the effect of Ca²⁺ in activating lymphocytes. This is associated with the activity of the adaptive immune system.

Furthermore, T cells may be activated or inactivated by modulating their bioelectric state. Activation occurs when ion channels are overexpressed and thus opened up to allow an inward current of calcium ions into the cells and a reverse current of potassium and chloride ions out of the cell. The net effect is a depolarization where the potential across the T-cell cell membrane is lowered. This has the following consequences: (1) the ion channels (and hence the associated ion currents) in the T-cell cell membrane preferentially cluster at the immunological synapse (i.e., where T cells bind to antigens), which is analogous to ion channel accumulation at neuronal synapses; (2) there is a memory effect for T-cell depolarization, as subsequent binding to antigens occasions inducement of a greater calcium ion current; (3) a similar phenomenon has been observed in NK cells in the innate arm of the immune system: depolarization across cell membranes via inwardly-flowing calcium ion currents can activate NK cells; and (4) silver ions can reversibly depolarize cell membranes by promoting a similar inward calcium ion current.

When Ca²⁺ is pumped into a lymphocyte, therefore, it signals the cell to change shape, activate enzymes, and turn on genes that express K+ and Ca²⁺ ion channels. This then causes the cell membrane to open up and pump K+ ion out of the cell creating a negatively charged lymphocyte (−85 mV).

Therefore, although Applicants do not intend to be bound by this theory, devices and methods according to the present invention function by two interacting mechanisms: (1) the production of antigens from pathogens that can be presented by lymphocytes; and (2) the stimulation of T cells by activation induced by ions generated by devices and methods according to the present invention.

For the ion-producing metal that is an alloy, the percentage of each metal in the alloy can depend on the result desired. Generally, however, when an alloy is used, the major metal in the alloy must be present in a proportion equal to or greater than 70% for optimum results in destroying pathogens. The goal is to destroy blood-borne pathogens using ions produced from these ion-producing metals that have been inserted either directly into the bloodstream or are inserted in an arrangement such that they come into contact with the bloodstream of a patient to be treated. Appropriate concentrations and durations of administration of metal ions can kill pathogens without affecting the cells of the patient such as erythrocytes (red blood cells) and leukocytes (white blood cells, including lymphocytes that play a key role in establishment and maintenance of the cellular and humoral immune response).

In one alternative according to the present invention, the ion-producing metal is an alloy of gold and silver in which the alloy comprises 70% or more of gold and 30% or less of silver. In another alternative according to the present invention, the ion-producing metal is an alloy of gold and silver in which the alloy comprises 70% or more of silver and 30% or less of gold. In still another alternative according to the present invention, the ion-producing metal is an alloy of gold, silver, and copper in which the alloy comprises 70% or more of gold and a total of 30% or less of silver and copper. In still another alternative according to the present invention, the ion-producing metal is an alloy of silver, copper, and gold in which the alloy comprises 70% or more of silver and a total of 30% or less of copper and gold. In still another alternative according to the present invention, the ion-producing metal is an alloy of silver, copper, and gold in which the alloy comprises 70% or more of copper and a total of 30% or less of silver and gold.

Typically, the electrode has between 1% and 99% each of gold, silver, and copper.

In other alternatives according to the present invention, the ion-producing metal can include one or more of platinum, iridium, and zinc. Therefore, the possible additional combinations of metals are within the scope of the present invention: (1) an alloy of gold and one or more of silver, copper, platinum, iridium, and zinc in which the alloy comprises 70% or more of gold, and a total of 30% or less of silver, copper, platinum, iridium, or zinc; (2) an alloy of silver and one or more of gold, copper, platinum, iridium, and zinc in which the alloy comprises 70% or more of silver and a total of 30% or less of gold, copper, platinum, iridium, or zinc; (3) an alloy of copper and one or more of gold, silver, platinum, iridium, and zinc in which the alloy comprises 70% or more of copper and a total of 30% or less of gold, silver, platinum, iridium, or zinc; (4) an alloy of platinum and one or more of gold, silver, copper, iridium, and zinc in which the alloy comprises 70% or more of platinum and a total of 30% or less of gold, silver, copper, iridium, or zinc; (5) an alloy of iridium and one or more of gold, silver, copper, platinum, and zinc in which the alloy comprises 70% or more of iridium and a total of 30% or less of gold, silver, copper, platinum, or zinc; and (6) an alloy of zinc and one or more of gold, silver, copper, platinum, and iridium in which the alloy comprises 70% or more of zinc and a total of 30% or less of gold, silver, copper, platinum, or iridium. Other combinations of metals can be used in alloys according to the present invention.

Use of devices and methods according to the present invention will greatly reduce exposure to life-threatening complications resulting from infection, and will also provide a substantial reduction in duration in time required for treatment. Methods and devices according to the present invention can be used at a doctor's office, at home, at a hospital or clinic, on a battlefield, or as a vacation extended treatment.

The system is fairly basic and simple in both its construction and use. A wire, either manufactured as part of a catheter or manufactured with an insulating cover is inserted into the patient's blood flow for the treatment of blood-borne pathogens. The details of the design depend on the characteristics of the patient, the particular pathogen being treated, and the duration of treatment. The duration and intensity of the release of ions is controlled by a proprietary control system or other suitable controlling unit; the control unit has within it a PCB (printed circuit board) with proprietary software that controls current, waveforms, and output for various programs for treating blood-borne pathogens, employing an external software program.

Typically, in operation, the device releases from about 2×10⁹ to about 7×10¹² ions per second for the treatment of blood-borne pathogens or stimulation of the immune system. In methods and devices according to the present invention, the voltage applied to generate the metallic ions is typically about 1.2 V or less. Preferably, the voltage applied is from about 0.5 V to about 0.92 V; in some alternatives, a voltage of 0.87 V or 0.89 V is employed. The voltage applied depends on the resistance of the tissue. Typically, the current employed is less than about 10 μA, more typically about 6 μA or less, preferably about 4.9 μA. In most applications, it is important to keep the voltage applied to generate the metallic ions at about 1.2 V or less; a voltage above that range tends to result in the binding of ions generated with chlorides in the bloodstream and to result in the formation of particles that lack therapeutic efficacy.

In one alternative, the device provides constant current with a cap on voltage. In another alternative, the device provides constant voltage with a cap on current.

Typically, the length of exposed metallic wire from the distal end of the fully exposed wire design is from about 0.1 inch (0.254 cm) to about 1.0 inch (2.54 cm). Typically, as further described below, the wire is skived to protect against breakage and for safety. Typically, the range of exposed surface area of the metallic wire for the proper release of ions is from about 0.0005 square inches (0.0032 cm²) to about 0.70 square inches (4.52 cm²). Typically, the electrode is placed into the venous system so that a length of the electrode of at least 0.5″ (1.27 cm) is within the venous system.

When a skived electrode is employed, it is preferable to protect the extended tip with a domed end. This allows a safer, more comfortable insertion placement of the electrode. The extended insulation beyond the last skived or open window that exposes the metal is, at a minimum, 0.5″ (1.27 cm) and have a domed end and also cover a blunt end where the actual tip of the wire can be seen.

As used herein, the term “patient” refers not only to a human, but also to mammals and also animals in general. Devices and methods according to the present invention can be used to treat pathogenic infections not only in humans, but also in socially or economically important animals including, but not limited to, dogs, cats, horses, sheep, cattle, pigs, rabbits, and goats.

As used herein, the term “wire” refers to a thin, straight, and rigid or flexible conductor that has the characteristics described above and further described in detail below. Typically, the wire is circular or oval in cross-section. The term “aleated” means completely coated or insulated except for a small portion. Typically, insulation of the insulated portions of the electrode is accomplished by use of electrical insulation for insulation of low-intensity direct current, typically silicone-based insulation suitable for medical use. The silicone-based insulation suitable for medical use is approved by the FDA or by comparable regulatory agencies in other countries.

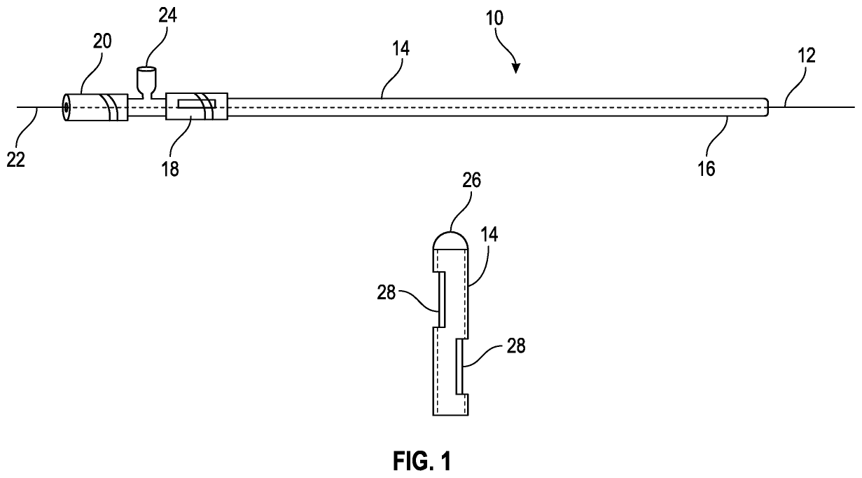

FIG. 1 is a front view of a device and method for killing pathogens, such as bacteria, virus, fungi, or protozoa, modulating the immune system, or creating a zone of inhibition to protect against microbial buildup on catheters or microbial infection at puncture sites of catheters in the blood of a patient according to an embodiment of the present invention. The device 10 includes a wire 12 that is protected by an insulated covering 14. The wire 12 and insulated covering 14 are inserted into the bloodstream of a patient via a catheter 16. As detailed above, the actual composition of the wire 12 can vary depending on the treatment desired. Once the wire 12 is in contact with the bloodstream of the patient, the wire 12 releases ions that kill pathogens which reside within the bloodstream that flows around the wire 12. The insulated covering 14 protects the wire 12 from being damaged. The insulated covering 14 also protects the patient from being injured by the wire 12 breaking off inside the patient's bloodstream and also ensures that the proper release of ions for therapy is delivered to destroy the pathogen or pathogens that constitute the therapeutic target. The proximal portion of the device has a male connector 18 connecting with a female connector 20. The connectors 18 and 20 protect the more sensitive components and provide threads or other mating mechanisms to allow them to be connected to the catheter 16. Within the male connector 18 is a copper pin 22 or a pin of another conductive material for transmission of the low density direct current by which the device 10 is connected to a power supply (not shown). The purpose of the pin 22 is to supply low intensity direct current to the device 10 which creates or accelerates the release of ions. The wire 12 in the blood will release ions on its own, but not at the rate or volume required to effectively destroy pathogens. The device can optionally include a side port 24 for introduction of a medication.

In an alternative for this device, the pin of conductive material, typically copper, can be replaced with a heat shrink connector that incorporates a circular solder ring. For the use of the heat shrink connector, two wires that are to be connected are placed in opposite ends of the connector so that the wires adjoin within the circular solder ring, heat is applied to the external connector surface which activates the solder ring to connect the wires, followed by the exterior plastic material shrinking down and encapsulating the connected wires.

FIG. 1 also shows a different embodiment of the same general idea. Rather than having an insulated covering 14 covering all of the wire 12 other than its tip, this embodiment has the insulated covering 14 covering all of the wire, with a domed cap 26 completing the protection of the wire 12. This embodiment has one or more cutouts 28, which expose the wire 12 to the bloodstream, and allow the wire 12 to release the desired ions. This second embodiment is more secure than the first, as there is no barren wire 12 within the blood stream.

The device of FIG. 1 generally works according to the following method. The electrode wire is annealed and insulated up to the exposed tip or cutouts (if the latter are present) so that no ions are released from any portion of the wire other than the intended release point, critical to the therapy, which is well into the vein and the flow of blood. This ensures that there will not be any exposed prior wire prior to the intended release point where ionic release is intended to occur, to prevent ionic release resulting in a less than ideal volume of ions released to reach and treat the target pathogen. Due to the rapid release of up to trillions of ions per second, the proximity of the free or bare metal or wire must be a minimum of 0.5″ (1.27 cm) or more beyond the tip of the introducer needle or catheter or from the entry point of the vein. This distance is chosen to prevent the insertion of an excessive length of an exposed metallic surface into the vein for ionic release.

It has been found that the volume of ions released by the wire is dependent on the length and/or surface area of the exposed metal, such that having the proper length of exposed wire, either in terms of its length or its surface area, is directly related to the potential or efficacy for killing the target pathogens. As such, a variety of lengths of exposed wire and variations on how this proper length is exposed are contemplated, because the lengths and surface areas of the exposed wire is critical to the actual ionic release mediated with low-intensity direct current (LIDC). It has been shown that different pathogens require different loads or releases or volumes of ions, similar to different doses of a pharmaceutical drug that may be required to kill specific pathogens; typically, for a pharmaceutical drug, these doses are expressed in mg, although the dosage will vary according to the particular drug, the infection being treated, pharmacokinetic factors such as kidney and liver function, and other factors well known in the art.

The present invention contemplates the use of three different application electrodes, each more or less efficient depending on the pathogen targeted. These electrodes include: a Central Venous Electrode (CVE), a Peripheral Venous Electrode (PVE), and a PICC Electrode (PICCE), which is an electrode placed within a peripherally inserted central catheter (PICC). The length of the annealed or insulated wire extending from the proximal or originating point of the male and female connectors is critical to proper placement and delivery of the metallic ions for the treatment of blood-borne pathogens. Therefore, the length of the aleated wire in the different electrode designs is different, but is generally from about 1.5″ (3.81 cm) to about 32″ (81.28 cm) (about 1.5″ (3.81 cm) to about 6″ (15.24 cm) for PVE; about 4″ (10.16 cm) to about 9″ (22.86 cm) for CVE; and about 6″ (15.24 cm) to about 32″ (81.28 cm) for PICC.

Devices according to the present invention, including, but not limited to the device depicted in FIG. 1, also rely on waveforms to enhance and increase safety during treatment with devices and methods according to the present invention. The background current waveforms from devices according to the present invention are delivered from the power supply to enhance blood flow to the area wherein the electrode is placed and enhance distribution of metallic ions within the venous system to enhance the efficacy of the therapy.

Typically, a device according to the present invention provides a range of current that generates from about 2.0×10⁹ ions per second to about 7.0×10¹² ions per second. As time passes, the current varies so that the number of ions generated per second ranges from about 2.0×10⁹ ions per second to about 7.0×10¹² ions per second, going up to about 7.0×10¹² ions per second, then slowly down to about 2.0×10⁹ ions per second, then slowly back up to about 7.0×10¹² ions per second, in a repeating cycle. The variable of how long it takes to ramp up and ramp down the volume of ions released is a variable that can be set from the device controller. This can be, for example, a fixed cycle of increasing the number of ions generated from about 2.0×10⁹ ions per second to about 7.0×10¹² ions per second and then decreasing the number of ions generated back to about 2.0×10⁹ ions per second at a preset interval, or, alternatively a variable cycle of increasing the number of ions generated from about 2.0×10⁹ ions per second to about 7.0×10¹² ions per second and then decreasing the number of ions generated back to about 2.0×10⁹ ions per second so that the duration of the increase of the number of ions generated and then the decrease of the number of the ions generated is varied. A number of alternatives exist for variable cycles; for example, the duration of a cycle can vary linearly, either increasing linearly or decreasing linearly from a fixed initial value. Alternatively, the duration can first increase and then decrease. Other alternatives for variable cycles are possible.

Crucial to the operation of the device is its use of phased output current. A timed or phased release of output current allows more a rapid release of metallic ions over certain periods of the treatment, and a slower release of metallic ions over the remaining periods of time during the duration of the treatment. One such example is to lower metallic ion release as factors as bacteremia (the concentration of a particular pathogenic bacterium such as Staphylococcus present in the blood) or viremia (the concentration of a particular pathogenic virus such as severe acute respiratory syndrome coronavirus 2 present in the blood) are improved during the course of therapy. Another example is to lower metallic ion release during hours of sleeping, both making the therapy more efficient and comfortable for the patient.

The rate of ionic release, and the particular cycle of release employed, can be varied as needed depending on the age, sex, and weight of the patient, the particular pathogen infecting the patient (bacteria, viruses, or fungi), the severity of the infection, particularly the concentration of the pathogen present in the blood, other drugs being administered to the patient, and factors such as liver and kidney function of the patient that may affect pharmacokinetics.

In some alternatives, patients to be treated by devices and methods according to the present application may be infected with more than one pathogen. For example, it is common for patients infected by an influenza virus or other respiratory virus to also acquire a secondary bacterial infection. This is more likely to occur in patients who have immunosuppression, such as can be induced by administration of anti-neoplastic drugs that suppress bone marrow function. In such cases, the rate of ionic release, and the particular cycle of release employed, can also be varied depending on the particular pathogens infecting the patient and the concentration of the pathogens in the blood of the patient, among other factors as described above.

The present invention also contemplates a combination catheter that allows for both the simultaneous release of metallic ions as described above and the administration of an intravenous antibiotic, antiviral agent, antifungal agent, or other medicament via a different or separate lumen within the same catheter for the treatment of blood-borne pathogens including, but not limited to, antibiotic-resistant bacteria. As stated above, the treatment can also accomplish the activation of the immune system and protection against microbial buildup on catheters or microbial infection at puncture sites of catheters. The combination catheter ranges from 12-26 gauge or sheath introducers up to 12 FR (French) to properly introduce electrodes. This range of catheter sizes allows for a range of electrode diameters to be inserted into the blood flow of the patient. This range of catheter sizes allows a range of electrode diameters in order to treat a range of patients including both youth and adults. The connection will allow a screw-on connector or a universal connector to accept the multiple available intravenous piggyback systems. The external or internal surface of the catheter has a spiral-wound metallic wire as described above that is designed for the specific ionic release necessary for the applicable therapy. The metallic wire can alternatively be arranged in a mesh, braided, or hexagonal pattern. The factors affecting the optimal ionic release are as stated above. Typically, in this alternative, the exposed spiral wire has a range covering a length of from about 0.5″ (1.27 cm) to about 2″ (5.08 cm) of the distal end of this form of the catheter with either a wide spiral or a tight spiral. The spiral can be either internal or external. In another alternative, the wire can be braided into the internal or external surface of the catheter. Typically, the distance of the braid from the end of the catheter is from about 0.5″ (1.27 cm) to about 2″ (5.08 cm) from the distal end of the catheter. In this alternative, the ranges of spiral or braided wire incorporated into the catheter are the ranges necessary to achieve the proper volume of ionic release as determined by the factors stated above with respect to the characteristics of the patient, the infection or infections affecting the patient, and other medications being administered to the patient. The basic design of this embodiment of the present invention provides that the inserted catheter has a spiral or mesh wound metallic wire as part of the construction of the catheter, allowing it to have the required flexibility so that it can be inserted into the venous system. The catheter also has enough metal exposed for the desired volume and rate of release of metal ions for treatment of the blood-borne infection. The catheter also has a connection point at the proximal end of the catheter to connect the source of the low-intensity direct current, such as a generator, to the catheter so that once the current is turned on the metal ions are released for distribution into the venous system. Additionally, such a single-lumen or multi-lumen catheter has a connection to an external intravenous antibiotic or therapy bag so that the desired solution can be administered through a lumen within the catheter and go directly into the venous system. Such a combination catheter according to the present invention allows the simultaneous release of metallic ions and the administration of an intravenous antibiotic, antiviral, or antifungal agent for the treatment of blood-borne pathogenic infections as described above. Typically, the dimensional range of the diameter of the spiral wound or braided wire is from about 0.001″ (0.00254 cm) to about 0.020″ (0.0508 cm).

The power supply used to supply the low-intensity direct current typically comprises a clamp to regulate input voltage, a regulator to regulate the actual current flowing to a control board (a printed circuit board (PCB)) that prevents excess voltage or current from flowing to the control board for its protection, and an output current and voltage regulator to protect the electrode and to prevent excessive ionic release by the electrode in order to provide an optimal therapeutic effect and to protect the patient from excess current.

The goal of the present invention is to combine technologies in order to allow devices and methods according to the present invention to be used to treat infections caused by a greater range of pathogens. The alternatives described above enable a user of the invention to customize the iontophoretic release of metal ions for the target pathogen or pathogens, along with considerations of other factors such as the age, weight, and sex of the patient, the severity of the infection or infections, or pharmacokinetic considerations affecting other drugs administered concomitantly with the use of devices and methods according to the present invention. This combination also lowers the production cost of the electrode thus making the therapy more affordable. This combination also provides a wider therapeutic window for treatment. This combination also makes the placement of the electrode safer and more comfortable to the patient. This combination also makes the use of the device less invasive for the patient.

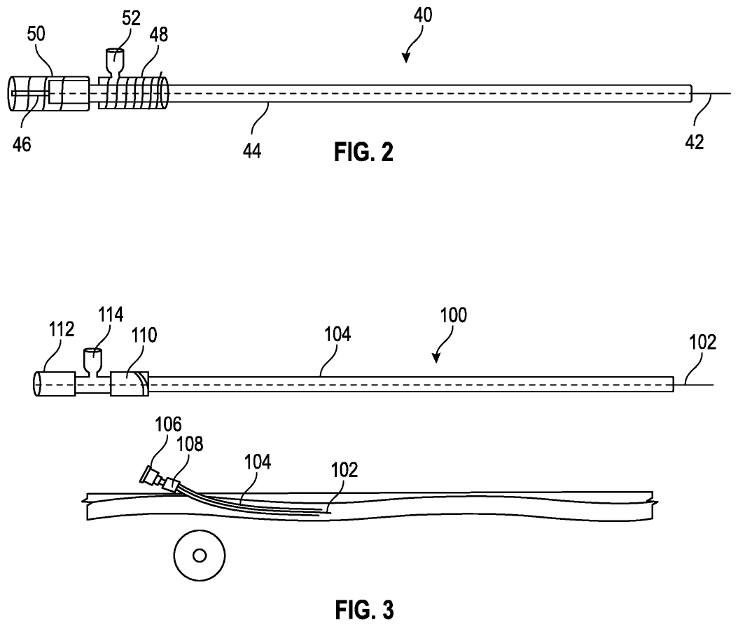

FIG. 2 is a front view of another embodiment of a device according to the present invention for killing bacteria, viruses, or fungi in the blood, modulating the immune system, or creating a zone of inhibition to protect against microbial buildup on catheters or microbial infection at puncture sites of catheters. The device 40 includes a wire 42 as in FIG. 1, an insulated covering 44 as in FIG. 1, a copper pin 46 or a pin of another conductive material as in FIG. 1, a male Luer lock or other connector 48, a female Luer lock or other connector 50, and, optionally, a side port 52 for the administration of intravenous antibiotics or other desired medications. The copper pin or pin of another conductive material can be replaced by a heat shrink connector incorporating a circular solder ring as described above.

FIG. 3 is a front view (top panel) and a side view (bottom panel) of another embodiment of a device according to the present invention showing insertion of the device into the arm of a patient. In the top panel, the device 100 includes a wire 102 as in FIGS. 1 and 2; the figure shows how the length of the wire 102 that is not covered by an insulated covering 104 can vary. In a preferred embodiment, the length of the wire 102 that is not covered by the insulated covering 104 can vary from about 0.1″ (0.254 cm) to about 1.0″ (2.54 cm). In FIG. 3, a Luer lock or other connector 106 is connected to an electrode 108. The electrode 108 rests on top of the patient's skin, while the insulated covering 104 and the wire 102 have been inserted into the patient's vein, such that the voltage and current delivered to the wire 102 can be electronically controlled to release metallic ions as described above to kill blood-borne pathogens. The width of the wire 102 and the insulating covering 104 can vary. The general range is from about 0.005″ (0.127 cm) to about 0.025″ (0.635 cm), while a preferred embodiment calls for about 0.010″ (0.254 cm) of insulation. The device of FIG. 3 further includes a male Luer lock or other connector 110, a female Luer lock or other connector 112, and, optionally, a side port 114 for the administration of intravenous antibiotics or other desired medications.

As can be seen from FIG. 3, the introduction of metal ions via insertion of a wire into the venous system of a patient is superior to the prior art of intravenous drip and iontophoretic release through skin applied pads, as those methods do not produce enough ions and also do not get the ions into the venous system in high enough volumes to be effective in denaturing, destroying, or otherwise inactivating the target blood-borne pathogens. Therefore, to effectively treat the target pathogens, it is typically required to have in excess of 2×10⁹ ions per second produced directly within the venous system.

Research by the inventors have shown, especially with certain pathogens, that the time for treatment may exceed 1-2 days, and that having the ionic release in the chest area can be superior and faster for therapies that require an extended period of time. To facilitate this method of treatment, devices and methods according to the present invention can rely on a peripherally inserted central catheter (PICC). The use of a PICC requires the user of the device to measure the proper length required for the patient and to cut it to the proper length during placement; thus, the electrode will also have to be modified.

The inventors have also discovered, without being bound by this theory, that the actual volume of ions release per second is important to the success of the resulting therapy. Metallic ions, such as, but not limited to, gold, silver, or copper, have a short lifetime within the body and the venous system. After a short period, their charge is neutralized and they are subsequently filtered and excreted from the body. Therefore, it is important that a certain level of concentration of metallic ions be achieved to have the most efficiency for treating the targeted pathogen; therefore, the iontophoretic constant release of metallic ions directly into the bloodstream in the range of from about 2×10⁹ ions/second to about 7×10¹² ions/second is preferred.

Research has also established that with a range of wire diameters, exposed wire of various lengths, annealed wire and volume of ions required, that to achieve the appropriate iontophoretic release of metallic cations with the variables stated, an output current from the device in the range from about 1.25 μA to about 6.0 μA of current is optimal for release of metallic cations for the treatment of blood-borne pathogens. Preferably, the current is about 4.9 μA.

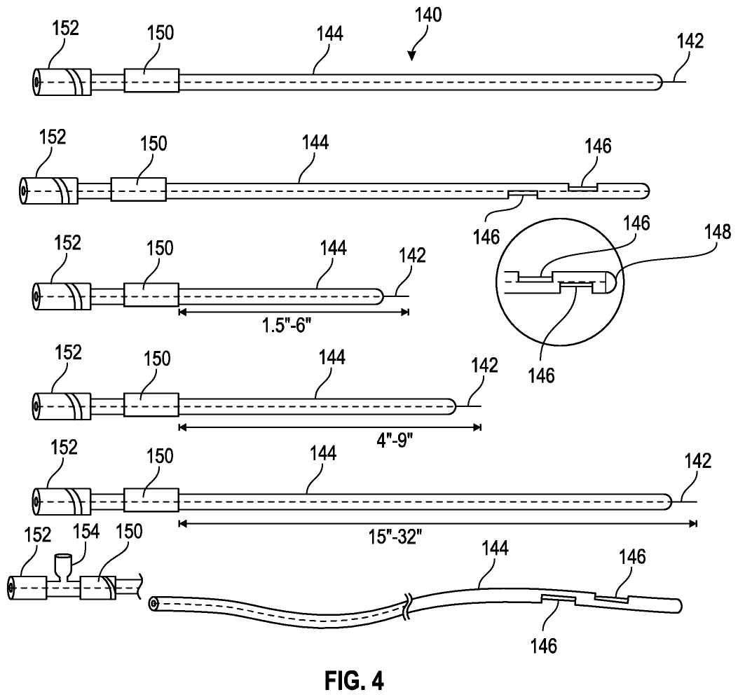

FIG. 4 is a series of front views of a device 140 for treatment of blood-borne pathogens in blood, activating the immune system, or creating a zone of inhibition to protect against microbial buildup on catheters or microbial infection at puncture sites of catheters. The device 140 includes a wire 142 and an insulating covering 144. FIG. 4 shows how the length of the wire 142 and the insulating covering 144 can be varied. FIG. 4 also shows that, when the wire 142 has been completely covered by the insulating covering 144 and a domed cap 148 except for one or more cut-outs 146, the length of exposed wire can be relatively equal to the exposed wire when the wire is allowed to trail from the end of the insulated covering 144. This figure also shows a number of variations in the length of the insulating covering 144 and the wire 142 for different end uses. The device of FIG. 4 also includes a male Luer lock or other connector 150 and a female Luer lock or other connector 152. The device of FIG. 4 can optionally include a side port 154 for the administration of intravenous antibiotics or other desired medications.

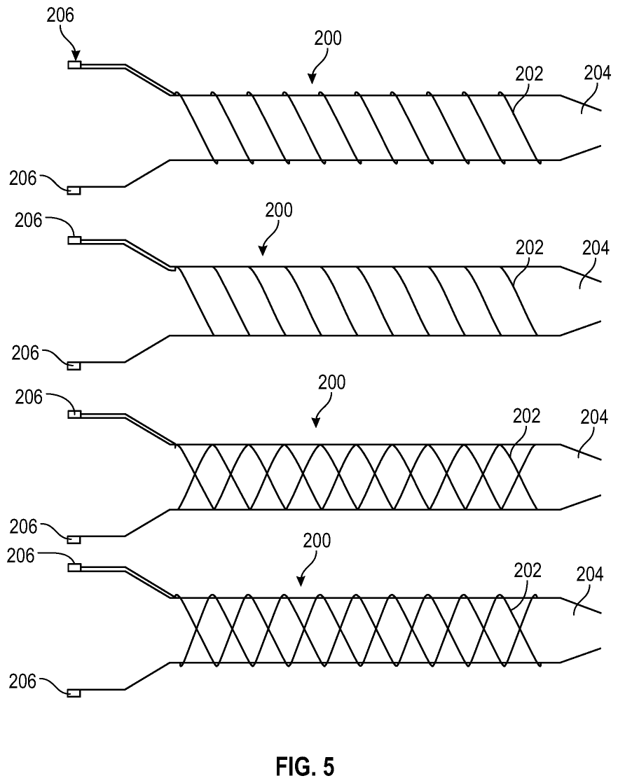

FIG. 5 is a series of front views of a device for killing antibiotic resistant bacteria or other blood-borne pathogens, activating the immune system, or creating a zone of inhibition to protect against microbial buildup on catheters or microbial infection at puncture sites of catheters according to the present invention. In FIG. 5, the device 200 includes a wire 202 that is coiled or braided around a catheter 204 and the entire unit inserted into a patient's bloodstream. Each end of the wire 202 is connected to a cap connector 206 which is in turn connected to a source of power as described above controlling the energization of the wire 202 for the release of metallic ions to effect treatment.

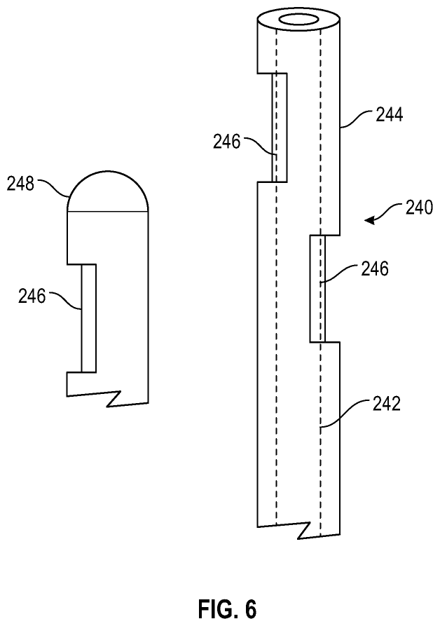

FIG. 6 is a series of front perspective views of a device for killing antibiotic resistant bacteria or other blood-borne pathogens, activating the immune system, or creating a zone of inhibition to protect against microbial buildup on catheters or microbial infection at puncture sites of catheters according to the present invention. FIG. 6 provides some detailed illustrations for a device 240 according to the present invention in which the insulated covering 244 covers the entire wire 242, other than for one or more cut-outs 246. The wire 242 is further retained within the insulated covering 244 by a domed cap 248 that seals the open end of the insulated covering 244.

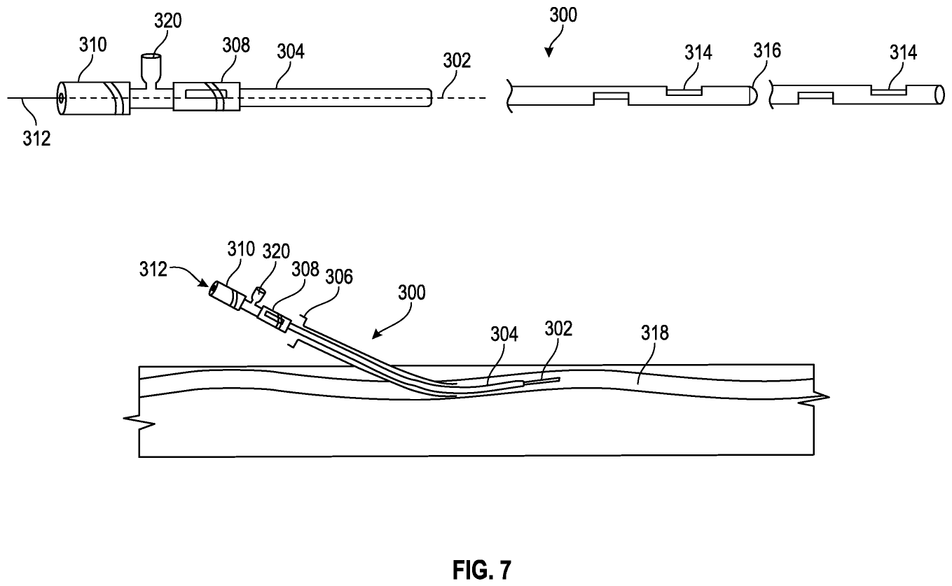

FIG. 7 is a series of front views of a device according to the present invention mounted with a catheter inserted into the vein of a patient. The device 300 includes a wire 302 that has been inserted into the vein of the patient and is protected by the insulated covering 304. A cap connector 306, male Luer lock or other connector 308 and female Luer lock or other connector 310 protect a copper pin 312 or a pin of another conductive material for connection to a power supply as described above. The insulated covering 304 can include one or more cut-outs 314 and can be retained by a domed cap 316. The device 300 is mounted into a catheter 318. The device 300 can further include a side port 320 for the administration of intravenous antibiotics or other desired medications.

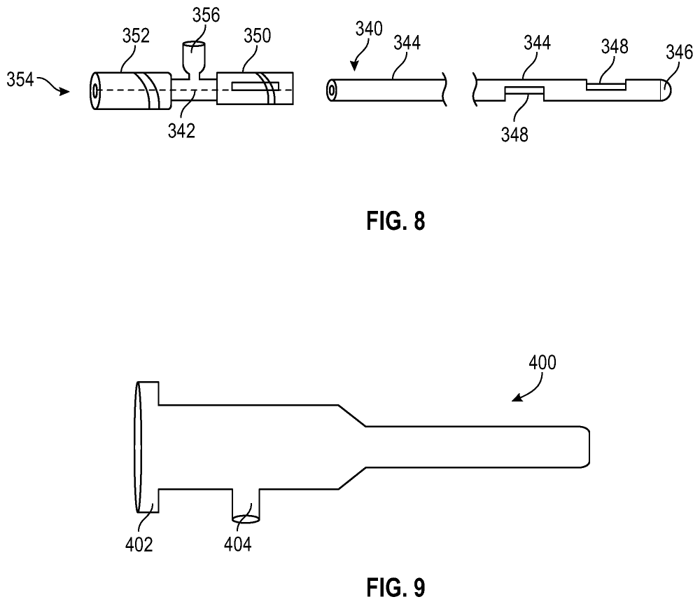

FIG. 8 is a series of front views of a PICC device according to the present invention. In this embodiment of the invention, the device 340 has a wire 342 surrounded by an insulated covering 344. The insulated covering 344 extends all the way to a domed cap 346. One or more cut-outs 348 provide access for the wire 342 to the bloodstream of the patient. The device 340 also has a male Luer lock or other connector 350 and female Luer lock or other connector 352 that protect a copper pin 354 or a pin of another conductive material. Optionally, the device has a side port 356 for the administration of intravenous antibiotics or other desired medications. Typically, the device is in the form of a single-lumen catheter such that the intravenous antibiotics or other desired medications can be administered through the side port 356. The insulated covering 344, which can be incorporated in the catheter, is larger in diameter than the wire 342 and typically tapers toward its distal end that is inserted into the vein of the patient. The side port 356 can be valved or covered.

FIG. 9 is a series of front views of an auxiliary lumen catheter for dual therapy according to the present invention. The auxiliary lumen catheter 400 has an hemostatic seal or side-port hemostasis valve 402 attached to close off space around the catheter 400. There is a side port 404 to allow for the administration of intravenous antibiotics or other desired medications. The inner diameter of the catheter 400 is greater than the outer diameter of an inserted electrode (not shown) so that anything added through the side port 404 will easily flow into the patient's bloodstream. This alternative of a device according to the claimed invention can be used for treatment of blood-borne pathogens in blood, activating the immune system, or creating a zone of inhibition to protect against microbial buildup on catheters or microbial infection at puncture sites of catheters.

A particularly preferred embodiment of the invention is described below. This particularly preferred embodiment is a device for treating a blood-borne pathogen within a bloodstream of a patient comprising:
(1) an iontophoretic wire, wherein the iontophoretic wire comprises an electrode comprising one or more metals, wherein the electrode comprises a vein portion and an exterior portion, the vein portion being inserted into a vein of the patient, the exterior portion being located outside of the vein of the patient, and wherein the vein portion is at least about 0.5″ (1.27 cm) beyond the tip of an introducer needle and an introducer catheter, the vein portion having a length of between about 0.2″ (0.508 cm) and about 1.0″ (2.54 cm), the vein portion having a vein portion surface area of between about 0.005 square inches (0.0322 cm²) and about 0.70 square inches (4.52 cm²);
(2) an exterior insulation that insulates the exterior portion of the electrode such that no ions are released from anywhere other than an intended release point, wherein the intended release point is located inside the vein of the patient, wherein the exterior insulation is an FDA-approved material selected from the group consisting of a synthetic and a plastic material, the exterior insulation having a durometer sufficiently low that the exterior insulation is sufficiently flexible to be placed safely within the venous system of the patient without causing significant irritation;
(3) a transformer to supply low-intensity direct current, wherein the transformer comprises one or more resistors and a transducer, wherein the transformer is in electrical contact with the iontophoretic wire; and
(4) a power supply in electrical connection with the transformer;
   wherein the device has an output current from about 1.25 μA to about 6 μA; and
   wherein the device produces at least 2×10⁹ ions per second within the bloodstream of the patient.

The one or more metals comprising the electrode are as described above.

In another alternative, the electrode is a two-part electrode with a first electrode part and a second electrode part. The first electrode part is a proximal end unit containing a conductive pin; the conductive pin comprises copper or another conductive material. The conductive pin is held within a Luer lock or other connector. The conductive pin allows the transfer of a low-intensity direct current to the iontophoretic wire from the power supply. The conductive pin can be tightened onto the second electrode part by use of a tightening device. The second electrode part has a length of exposed wire at its distal end.

In an alternative, the pin of conductive material, typically copper, can be replaced with a heat shrink connector that incorporates a circular solder ring as described above.

In alternative embodiments, the tightening device can be a set screw or a sufficient quantity of biocompatible conductive adhesive. Biocompatible conductive adhesives are described in U.S. Pat. No. 8,660,645 to Stevenson et al.

In other alternative embodiments, the length of exposed wire at the distal end is a bare wire, such as, but not limited to, a silver wire or a skived window exposing the wire.

In additional alternative embodiments, the wire can be tempered and/or annealed for proper ionic release and for the safety of usage of the wire within the venous system.

In still other alternative embodiments, a device according to the present invention can comprise a combination catheter for dual therapy, wherein the combination catheter allows the simultaneous release and administration of a quantity of metallic ions sufficient to treat blood-borne pathogens and a quantity of another intravenous medication, such as an antibiotic, another antibacterial agent that can be administered intravenously, an antiviral agent that can be administered intravenously, or an antifungal agent that can be administered intravenously. The metallic ions and the additional intravenous medication are administered to treat blood-borne pathogens such as, but not limited to, antibiotic-resistant bacteria. In these alternative embodiments, the gauge of the device typically ranges from 12 gauge to 26 gauge to properly introduce electrodes. This range of catheter sizes allows for a range of electrode diameters to be inserted into the blood flow of the patient. This range of catheter sizes allows a range of electrode diameters in order to treat a range of patients including both youth and adults. The device includes an intravenous connection that allows a universal connector as is conventionally used in the art to accept multiple available intravenous piggyback systems. In this alternative, an external surface of the catheter comprises a spiral wound metallic wire, such as, but not limited to, a silver wire, where the exposed spiral wire has a length in the range from about 0.5″ (1.27 cm) to about 2.0″ (2.54 cm), and a spiral diameter in the range from about 0.001″ (0.00254 cm) to about 0.1″ (0.254 cm). The interior diameter of the catheter is greater than the outside diameter of the inserted electrode, allowing the added intravenous administration as described above, such as an antibiotic, to easily flow down and around the electrode and thus flow into the bloodstream of the patient.

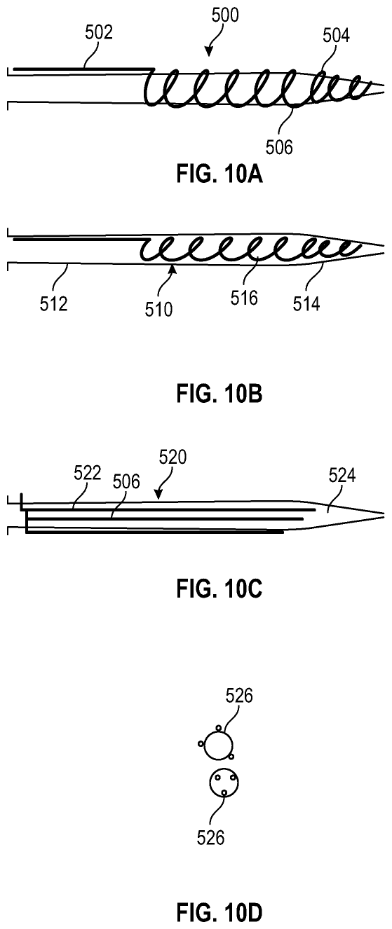

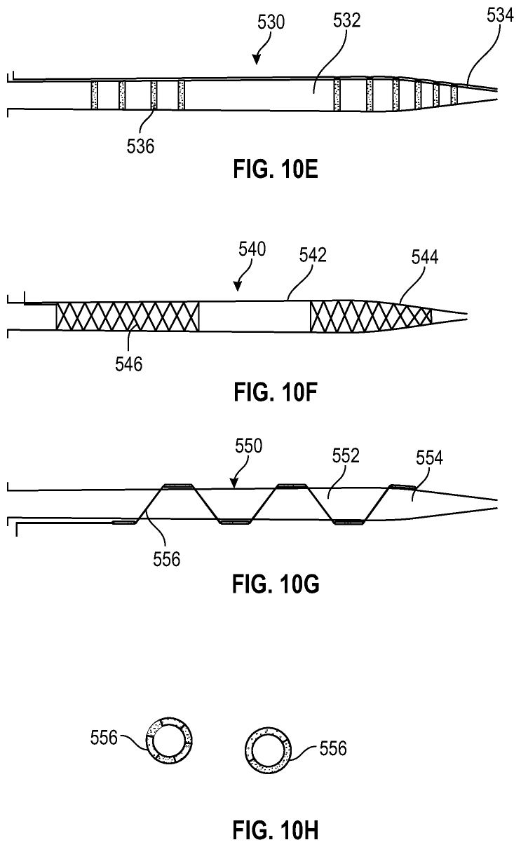

FIG. 10 is a diagram of several catheter designs for a “zone of inhibition” that deliver positively charged ions to stimulate the immune system and to kill pathogens in the blood. FIG. 10A has external spiral-wound wiring. FIG. 10B has internal spiral-wound wiring. FIG. 10C has straight longitudinal wiring, with three to five wires that are either internally exposed, externally exposed, or both internally and externally exposed; FIG. 10D is an end view of the catheter of FIG. 10C showing the two alternatives (externally exposed at the top, internally exposed at the bottom). FIG. 10E has rings at the distal end, the proximal end, or both; all rings can be anode, or anodes and cathodes can alternate every other ring, or the catheter can be used with an external catheter cathode, such as used in an EKG path; FIG. 10F has a hex design, distal, proximal, or both, with a proximal external surface, and a distal internal or external surface, or with a proximal internal surface and a distal internal or external surface, with a cathode externally applied, such as used in an EKG path; FIG. 10G has periodically exposed sections of wire from the proximal end to the distal end, or at the distal end only, with windows from 1 mm to 10 mm in length and from 35°-180° exposure; and FIG. 10H shows end views of the arrangements (i) with 3 30° windows; and (ii) with 1 180° window.

In the catheter design depicted in FIG. 10A, the catheter 500 has a wider proximal section 502 and a narrowing distal section 504 and is wrapped with spiral-wound wiring 506.

In the catheter design depicted in FIG. 10B, the catheter 510 has a wider proximal section 512 and a narrowing distal section 514 and includes internal spiral-wound wiring 516.

In the catheter design depicted in FIG. 10C, the catheter 520 has a wider proximal section 522 and a narrowing distal section 524 and includes wiring 526 that is either internally exposed, externally exposed, or both internally exposed and externally exposed.

In the catheter design depicted in FIG. 10D, which is an end view of the catheter design depicted in FIG. 10C, the top panel shows the wiring 526 that is externally exposed, while the bottom panel shows the wiring 526 that is internally exposed.

In the catheter design depicted in FIG. 10E, the catheter 530 has a wider proximal section 532 and a narrower distal section 534 and includes rings 536. The rings 536 can be located at the proximal section 532, the distal section 534, or both the proximal section 532 and the distal section 534. The rings 536 can be anodes for all rings, or can alternate between anodes and cathodes; alternatively, an external catheter cathode such as an EKG patch can be included.

In the catheter design depicted in FIG. 10F, the catheter 540 has a wider proximal section 542 and a narrower distal section 544 and includes wiring 546 arranged in a hexagonal pattern. The wiring 546 can be located at the external surface of the proximal section 542 and either at the external or internal surface of the distal section 544; alternatively, the wiring 546 can be located at the internal surface of the proximal section 542 and either at the external or internal surface of the distal section 544. As in the catheter design depicted in FIG. 10E, alternatively, an external catheter cathode such as an EKG patch can be included.

In the catheter design depicted in FIG. 10G, the catheter 550 has a wider proximal section 552 and a narrower distal section 554 and includes wiring 556 with periodically exposed sections of wire, either from the proximal end to the distal end or at the distal end only, providing windows from 1 mm to 10 mm in length and from 30° to 180° of exposure.

In the catheter design depicted in FIG. 10H, which is an end view of the catheter design depicted in FIG. 10E, the left panel shows the wiring 556 with three 30° windows while the right panel shows the wiring 556 with one 180° window.

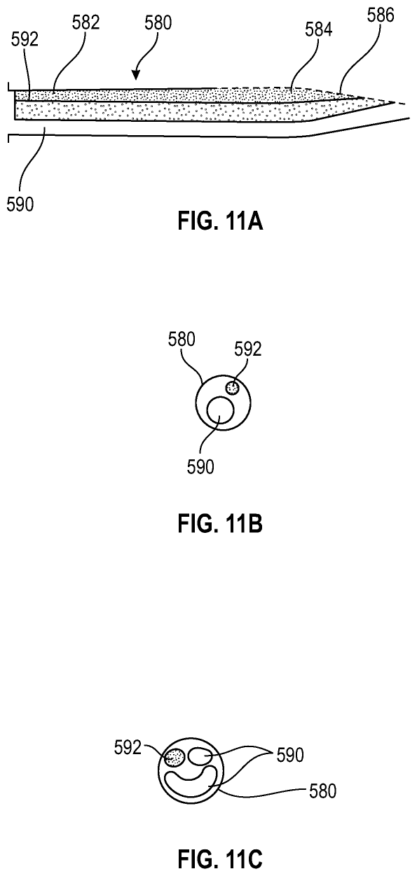

FIG. 11 shows alternatives for designs of a catheter according to the present invention using either a single- or multi-lumen design that could be either intended for peripheral, central, midline, or PICC insertion. FIG. 11A shows: (i) a side view of a dual lumen with metallic alloy implanted into the catheter with skived windows at the distal end for ionic release to: (A) create a zone of inhibition (antimicrobial activity); and (B) release positively charged ions (cations) to stimulate the immune system utilizing an external cathode such as used in an EKG path; and (ii) an end view of the single lumen and electrode; and FIG. 11B shows an end view of a similar multi-lumen catheter.

FIG. 11A shows a side view of a catheter 580 with a wider proximal section 582 and a narrower distal section 584 with a skived window 586 at the distal end with a dual lumen 588 with an open section 590 and a metallic alloy 592. FIG. 11B is an end view of the catheter of FIG. 11A.

FIG. 110 is an end view of a similar multi-lumen catheter with two open lumens and one lumen for insertion of the metallic alloy. The catheter 600 has a first open lumen 602, a second open lumen 604, and a third lumen 606 for insertion of the metallic alloy 608.

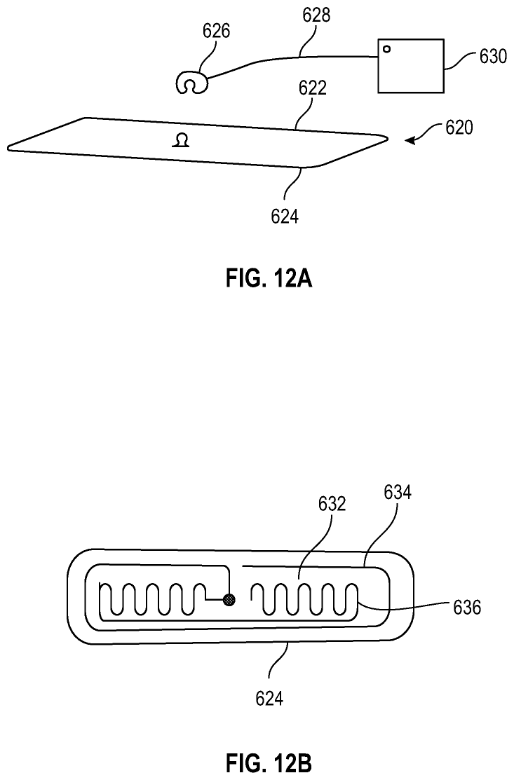

FIG. 12 is a schematic diagram of a wound care or burn care bandage, also described as a patch, according to the present invention. FIG. 12A shows a patch of varying size for application to an area of skin that incorporates into the dermal side of the patch (the side that comes into contact with the skin), an ion-releasing metallic alloy as described further below. The ion-releasing metallic alloy receives current when connected to a low-intensity direct current generator as described further below. FIG. 12B shows the bottom side of the wound care or burn care bandage, with the incorporated ion-releasing metallic alloy showing the cathode and anode.

In FIG. 12A, the patch 620 has a top side 622 and a bottom side 624. The top side 622 has a snap connector 626 attached; the snap connector is connected to a wire 628 which in turn is connected to a power supply 630 to provide low-intensity direct current to the patch 620.

In FIG. 12B, the bottom side 624 of the patch has a medicated coating incorporating a metallic alloy 632 to release metallic ions as described above when low-intensity direct current is applied to the metallic alloy. The metallic alloy has a cathode 634 and an anode 636. The material to be used on the dermal side of the patch is typically a hydrogel. A hydrogel is a network of crosslinked polymer chains that are hydrophilic, sometimes found as a colloidal gel in which water is the dispersion medium. A three-dimensional solid results from the hydrophilic polymer chains being held together by cross-links. The crosslinks which bond the polymers of a hydrogel fall under two general categories: physical and chemical. Physical crosslinks consist of hydrogen bonds, hydrophobic interactions, and chain entanglements (among others). Because of the inherent cross-links, the structural integrity of the hydrogel network does not dissolve as a result of the high concentration of water. Hydrogels are highly absorbent (they can contain over 90% water) natural or synthetic polymeric networks. Other alternatives for materials used on the dermal side of the patch can be used and are known in the art.

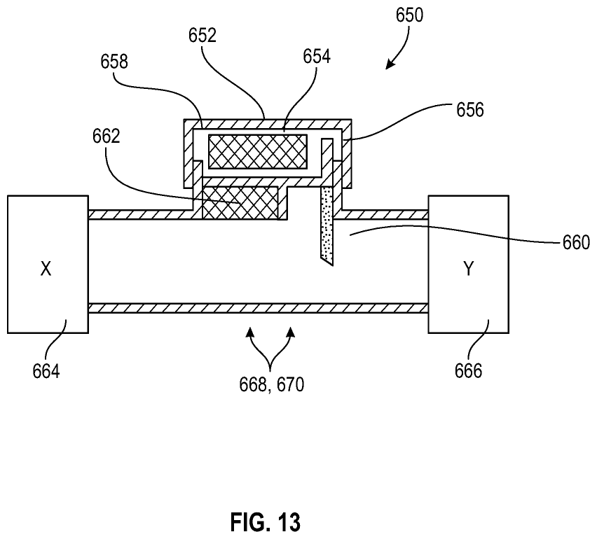

FIG. 13 depicts an alternative embodiment of the invention intended for incorporation into an intravenous line or other device intended to supply a fluid, such as, but not limited to, plasma, medications, nutritional supplements, or electrolytes, to a patient.

In FIG. 13, the device 650 has a hubcap 652 to cover the device. In the interior of the device 650 is located a cell battery 654 and a printed circuit board 656 to control the current output. The device 650 also includes a cathode wire 658 and an anode wire 660. The cathode wire 658 is in electrical contact with a fluid-absorbing material 662 that is also in electrical contact with the anode wire 660 to provide an electrical circuit when fluid flows through the device 650. The device 650 also includes a first connector 664 and a second connector 666 to enable placement of the device 650 into an intravenous line or other device to supply fluid to a patient as described above; the connectors are shown as “X” and “Y” in FIG. 13. The first connector 664 and the second connector 666 can be female or male Luer locks, intravenous hose clamps, intravenous hose connectors, or other connectors conventionally used in the art. The first connector 664 and the second connector 666 are joined by an anode metallic pin 668 and a cathode metallic pin 670.

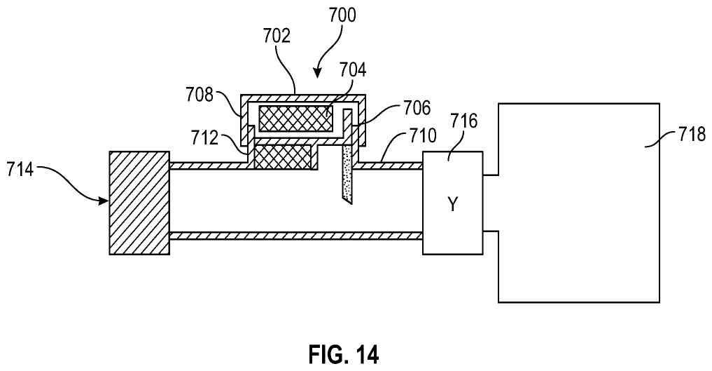

Another version of this device is shown in FIG. 14. This version of the device has the device attached to a fluid source that enables the production of metallic ions without insertion of the device into an intravenous line. In the version of the device shown in FIG. 14, the device 700 has a hubcap 702 to cover the device. In the interior of the device 700 is located a cell battery 704 and a printed circuit board 706 to control the current output. The device 700 also includes a cathode wire 708 and an anode wire 710. The cathode wire 708 is in electrical contact with a fluid-absorbing material 712 that is also in electrical contact with the anode wire 710 to provide an electrical circuit when fluid flows through the device 700. The device 700 also includes a solid cap 714 and a connector 716. The connector 716 is connected to a fluid source 718. The fluid source 718 provides distilled water, physiological saline, or another biologically compatible aqueous solution to enable the device 700 to produce metallic ions. The solid cap 714 and the connector 716 are joined by an anode metallic pin 720 and a cathode metallic pin 722.

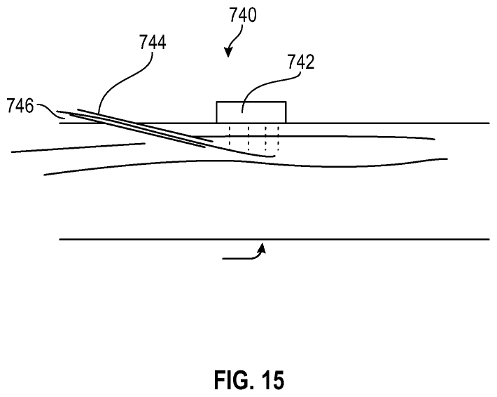

Yet another embodiment of a device according to the present invention is shown in FIG. 15. The device of FIG. 15 is intended to transmit metallic ions as described above into the vein of a patient via a temporarily implanted stylet or catheter.

In the version of the device shown in FIG. 15, the device 740 includes a generator 742 attached to the skin of a patient. The generator 742 transmits a current through an implanted stylet 744 with a wire 746 to produce metallic ions as described above. The implanted stylet 744 is typically implanted into a vein of a patient, such as in the arm of the patient. Instead of a cable providing the current from the generator 742 to the stylet 744, typically the generator 742 is attached to the skin of the patient and transmits the signal through the skin to the implanted stylet 744, which will activate the release of metallic ions.

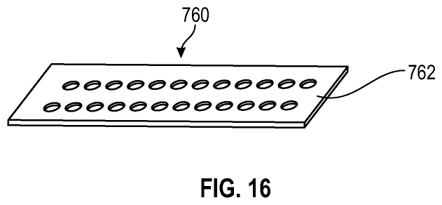

Yet another embodiment of a device according to the present invention is shown in FIG. 16. The device of FIG. 16 is a chewable gum that continuously releases silver or metallic ions to be quickly absorbed into the bloodstream via the mouth for health reasons or to provide a supplement.

In the device of FIG. 16, the device 760 comprises a wafer or layer 762 of a chewable gum that has particles of a metal or metallic alloy as described above incorporated therein, which are released on chewing, with the hydrolytic action of saliva enabling the release of the particles so that they are absorbed into the bloodstream where ions are released from the particles for therapeutic or immunostimulatory activity.

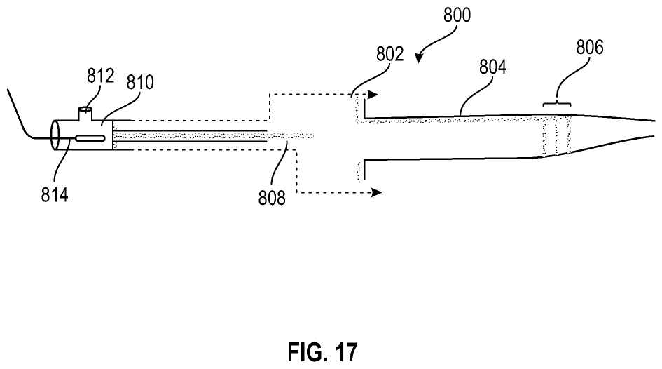

Still another embodiment of a device according to the present invention is shown in FIG. 17. In the device of FIG. 17, the catheter can be a single-lumen or multi-lumen catheter that can be used with a generator as described above or as a device for supplying intravenous fluids in conjunction with the release of metallic ions as stimulated by the generator as described above. The premise behind this embodiment is that the cathode, with 1 to 5 rings, is built into the catheter so that there is no need for an externally applied patch to complete the circuit with the generator. This provides a more constant resistance and assures a steadier, more constant delivery of voltage from the generator. In this alternative, the anode is constituted by a stylet that is inserted into the venous system via the catheter. In this alternative, there is a metallic ring built into the catheter that is attached to the surface point at the proximal end of the catheter. Within the connector hub of the anode stylet, there is a metallic surface area so that once the stylet is locked down via this connector it makes electrical contact with the catheter cathode completing the circuit.

In the device of FIG. 17, the device 800 includes a catheter 802 with an internally run wire 804 and one to five external conductive rings 806. The device 800 further includes an insulated wire 808 comprised of a metal or metallic alloy as described above. The device further includes a connector 810 in operable contact with the catheter, which optionally has a side port 812 for the administration of intravenous antibiotics or other desired medications. Inside the connector 810 is a wire 814 for connection to the generator. The device 800 is depicted with a single-lumen catheter, but, alternatively, a dual-lumen or multi-lumen catheter can be used.

In devices according to the present invention, the metal alloy as described above can be incorporated into a catheter in a number of alternative ways. The catheter can be a central venous catheter, hemodialysis central venous catheter, a peripheral catheter, a PICC (peripherally inserted central catheter), a mid-line catheter, a urinary catheter, or a drainage catheter. The alternatives are as follows: (1) a wire exposed both internally and/or externally; (2) a wire coiled throughout the catheter, internally and/or externally; (3) a wire running the length of the catheter, internally and/or externally; (4) a wire exposed only at the distal tip of the catheter, internally and/or externally; (5) a wire exposed only at the surface of the skin, internally and/or externally; (6) a wire exposed in multiple locations; (7) a wire arranged in a cuff to protect a puncture site; the cuff is situated on the catheter and slides, but will not go into the insertion site for the catheter; (8) a wire arranged in rings or bands with periodic spacing down the length of the catheter; (9) a wire that is spiral wound, in a hex design, internally and/or externally; or (10) a wire arranged with periodic exposure of the wire down the length of the catheter.

Other alternatives in devices according to the present invention include: (1) a catheter wrapped in wire externally to control the release of the metallic ions; (2) a coated catheter where a conductive material has metallic particles within (such as, but not limited to, silver particles) arranged such that when current is run through the coated catheter, it releases metallic ions (such as, but not limited to, silver ions).

In devices and methods according to the present invention, the metallic ions that can be released by device and employed in the method is typically an ion of copper, silver, gold, platinum, iridium, tin, osmium, palladium, or osmium. When the metallic ion is an ion of copper, the ion can have an oxidation state of +1 (cuprous ion) or +2 (cupric ion). When the metallic ion is an ion of silver, the ion can have an oxidation state of +1, +2, or +3; typically, the ion has an oxidation state of +1. When the metallic ion is an ion of gold, the ion can have an oxidation state of +1, +2, +3, or +5; typically, the ion has an oxidation state of +1 or +3. When the metallic ion is an ion of platinum, the ion can have an oxidation state of +1, +2, +3, or +4; typically, the ion has an oxidation state of +1 or +3. When the metallic ion is an ion of iridium, the ion can have an oxidation state of +1, +2, +3, +4, +5, +6, +7, +8, or +9; typically, the ion has an oxidation state of +3 or +4. When the metallic ion is an ion of tin, the ion can have an oxidation state of +2 or +4. When the metallic ion is an ion of palladium, the ion can have an oxidation state of +2, +3, or +4; typically, the ion has an oxidation state of +2. When the metallic ion is an ion of osmium, the ion can have an oxidation state of +1, +2, +3, +4, +5, +6, +7, or +8; typically, the ion has an oxidation state of +2, +3, +4, or +8.

In methods and devices according to the present invention, the voltage applied to generate the metallic ions is typically about 1.2 V or less. Preferably, the voltage applied is from about 0.5 V to about 0.92 V. The voltage applied depends on the resistance of the tissue. Typically, the resistance of the tissue is from about 5×10⁴ ohms to about 3×10⁵ ohms, more typically from about 1×10⁵ ohms to about 2×10⁵ ohms. Typically, the current employed is less than about 10 μA or less, more typically about 6 μA or less, preferably about 5 μA or less, most preferably about 4.9 μA. In most applications, preferably, the minimum output current is about 1.25 μA. In most applications, it is important to keep the voltage applied to generate the metallic ions at about 1.2 V or less; a voltage above that range tends to result in the binding of ions generated with chlorides in the bloodstream and to result in the formation of particles that lack therapeutic efficacy.

In devices according to the present invention as described above, the generator or other power source typically has the ability to adjust the current based on the therapeutic application. As detailed below, the current can be held constant, alternatively, the voltage can be held constant. The device automatically can hold the current constant. The firmware uses Ohm's law to maintain the current at, for example, 4.9 μA, but, if the resistance of the body, which is typically less than about 200 kΩ, rises to a value such that the output voltage must go higher than 0.87 V, 0.89V, or 0.92 V to maintain this value of the current, then the device will hold the output voltage at 0.87 V, 0.89 V, or 0.92 V and, dependent on the resistance, will lower output current, so that therapy remains constant, just at a lower output current and thus at a lower rate of ionic release.

In devices according to the present invention as described above, the device typically has the capability of wireless communication with a smartphone, employing NFC connectivity. NFC is a set of communication protocols for communication between two electronic devices over a range of about 4 cm (1.5 inches) or less. NFC offers a low-speed connection with a simple setup. NFC is standardized in ECMA-340 and ISO/IEC 10892. Alternatively, the NFC connectivity can be replaced with a PCB modem to be activated on a cellular service to allow both incoming and outgoing external communication. The NFC allows a smartphone apparatus to come near the device to upload or download data, change program parameters, and activate and start a therapy session. In another alternative, communication could be via Bluetooth. Other alternatives for communication are known in the art.

In devices according to the present invention as described above, the device typically also has the capability of wireless communication via a cellular phone system to download and upload data, firmware, or other information. The firmware can be programmed to control or adjust the pulse, frequency, current, voltage, pattern of oscillation, burst, or variable time associated with the administration of therapeutic ions to the patient. The information that can be downloaded to the device for modification of the function of the device can include, but is not limited to, the age of the patient, the ethnicity of the patient (particularly where relevant with respect to susceptibility to a particular disease or condition), the weight of the patient, genetic information such as the HLA haplotype, which can affect susceptibility of the patient to particular infective agents, and relevant medical history of the patient, such as the occurrence of diabetes, the occurrence of hypertension, a history of tobacco smoking, a history of alcohol use, or other relevant medical history that may affect susceptibility to a particular disease or condition, and the particular disease or condition that is to be treated or prevented by use of a device according to the present invention, such as, but not limited to stimulation of leukocytes to promote an immune response, severe acute respiratory syndrome coronavirus 2, infection, HIV infection, hepatitis A infection, hepatitis B infection, hepatitis C infection, dengue virus infection, Zika virus infection, bacterial infection, or fungal infection. This data transfer is fully compliant with HIPAA such that no personal information is collected or stored that would enable anyone using or otherwise working with the device to identify the particular patient. The relevant information described above would be input before starting the unit so that, for the unit session, the unit will store the average voltage, current, and resistance, and the length of therapy. The relevant information is typically input by NFC connectivity, although other connectivity routes can alternatively be used, as described herein. This input is used to start the operation of the device. Subsequent to use of the unit, the unit can upload all stored data from the session to a central server to enable the operator of the central server to gather significant global data from use of the device to determine the uses being made of the device including the disease, condition, or other indication being treated, the outcomes, and other information that will enable verification and possible modification of the technology. Again, this information transfer will be HIPAA-compliant with respect to the collection of data such as age, sex, ethnicity, the pathogen to be treated, with these parameters, to the extent necessary, to be linked to the actual time of therapy, the resistance, voltage, and current over that time period, in order to seek recognizable trends for pathogenic conditions and possible differences in types of individuals treated with the device. This information can be used to improve the efficiency of treatment and therapeutic results and will be anonymized so that individuals cannot be identified as the result of the transmission, analysis, or other use of the data. Typically, the device includes firmware to control or fix the output amperage while allowing resistance to control output voltage, until or when the resistance reaches a point that the output current, based on Ohm's law, would have to rise above the maximum allowed output current, at which point the firmware locks in the maximum output voltage and then lowers the output current based on resistance. At a certain point, which is controllable by the operator of the device, an alarm can notify the patient or an attendant that resistance is too high and the output current has hit a low set point, meaning that optimum release of ions for therapeutic activity is not occurring at that point.

In other alternatives, a device according to the present invention can be portable.

In devices according to the present invention as described above, the device typically also has the capability of providing different waveforms or of providing an oscillating or pulsing signal. With respect to signal pulsing, preferably the rate of pulsation is sufficiently fast so that the central nervous system (CNS) or the peripheral nervous system (PNS). Preferably, the rate of pulsation is also sufficiently fast to reduce or eliminate the risk of thrombosis. In another alternative, burst waveforms can be used. In yet another alternative, oscillating waveforms can be used. In yet another alternative, the current employed can be ramping or timed. Typically, when pulsing is employed, the rate of pulsing is sufficiently rapid so that the central nervous system (CNS) or the peripheral nervous system (PNS) does not recognize it and also is sufficiently rapid to suppress thrombosis. Typically, pulse widths range from about 50 microseconds to about 1000 microseconds. When burst waveforms are employed, combinations of pulses are combined with frequencies in rapid order to stimulate the immune system. An example would be five 1-microsecond pulses with a 1-microsecond interpulse interval at 500 Hz, applied at a 40-Hz frequency with passive regeneration of the charge balance at the end of the pulse burst. Frequencies employed can range from about 40 Hz up to about 10 kHz. Typically, a frequency of about 10 kHz is preferred for the background current in order to enhance blood flow to reduce the risk of phlebitis and to enhance ion signal distribution intravenously. When oscillating waveforms are used, the temporal pattern of application of current is similar to what is described above with respect to burst waveforms but can also include changes in current intensity to vary the stimulation patterns. An example would be an oscillating current from 3.0 μA to 4.9 μA for a specified period of time, then oscillating from 1.5 μA to 3.0 μA for the remaining time. This could be used in late therapy, for example with the lower oscillating current (a maximum of 3.0 μA) to be used in the evening or when the patient would be asleep. In one alternative, an optimum would be to oscillate over a 16-hour period followed by an 8-hour oscillating setting. When ramping or timed waveforms are used, the current is slowly ramped up in a linear fashion and then ramped down in a linear fashion to control the current delivered. Typically, this is done over a period of from about 5 seconds to about 10 seconds. For example, 5 to 10 seconds would be used to ramp up to full power, then 5 to 10 seconds to ramp down. In general, these alternatives would be programmable by a technician or other operator in order to optimize the efficacy of treatment. Other alternatives for the use of ramping or timed waveforms can be used.

In some alternatives according to the present invention, the system is programmable, and is able to program different settings for current application to take into account factors such as, but not limited to, time of day, body position, site of application, age, sex, and weight of the patient, the particular disease or condition to be treated, its severity, other conditions affecting the patient, or other factors.

In one alternative, the system provides constant current with a cap on voltage. This means that the system will maintain a set constant current output as long as the impedance of the system does not reach the point where the voltage cap is reached. Should the voltage reach the cap, the system will then reduce or allow the output current to decrease so as not to surpass the set capped voltage.

In another alternative, the system fixes the output voltage at a value from about 0.55 V to about 0.95 V. Preferably, in this alternative, the output voltage is fixed at about 0.92 V; in some alternatives, a fixed output voltage of 0.87 V or 0.89 V can be employed. The initial output current is at about 6 μA or less, typically at about 4.9 μA. In the event that resistance rises such that the increase in resistance would require the voltage to exceed about 0.95 V, then the voltage is capped and the output current is lowered to maintain the output voltage at a value no greater than about 0.95 V, preferably at about 0.92 V.

In yet another alternative, a second, high-frequency background signal, which overlaps with the constant current with a cap on the voltage, is introduced to enhance blood flow in order to enhance ion distribution and reduce the risk of phlebitis. The frequency of the high-frequency background signal is as stated above.

Another embodiment of the present invention is a small metallic ion generator to produce a solution containing metallic ions for various purposes, including treatment or prevention of bacterial, viral, or fungal infections, stimulating the immune response, or providing a zone of inhibition around a catheter placement to protect against the development of microbial biofilm or infection from the puncture site. The device incorporates a fixed small cell battery which is attached to a small printed circuit board that controls both the output voltage and output current to one or more permanently attached metal or metal alloy anodes and to a single cathode. From one to four anodes can be attached. The cathode is covered in a sponge-type material such as Porex from Porex Filtration Group (Fairburn, GA). The generator has a twist connector around the open end of the exposed anode and covered cathode which will allow for various attachments to be placed which will hold fluid that will be treated with metallic ions generated by the generator. Once a fluid-containing container is contacted to the metallic ion generator, the fluid will complete the contact between the anode and the cathode and allow for the controlled release of metallic ions within the fluid, creating an antimicrobial solution that can be used for various purposes. These purposes include, but are not limited to, a solution to be applied to the skin for treatment of conditions such as acne, a solution to be administered by a nebulizer for nasal therapy or lung therapy, or a solution to be swallowed or used as a mouth rinse. The antimicrobial solution can be used in multiple other uses. Typically, in this device, the battery is a 1.5 V to 3.5 V lithium cell battery. The dimensions of the battery typically range from about 0.5 cm to about 3.0 cm in diameter. The printed circuit board has a surface area of 5 cm² or smaller. The printed circuit board functions similarly to those described above and typically produces a fixed current outflow of 4.9 μA with a cap of 0.92 V; in some alternatives, a voltage of 0.87 V or 0.89 V is employed. Other alternatives for current and voltage are possible.

Another embodiment of the present invention is a battery-powered, reusable or disposable device used to provide a constant current and a capped voltage to a component such as the stylet of FIG. 14, above, for the purpose of controlling the continuous release of metallic ions. The metallic ions whose release is triggered by the battery-powered device can be generated from an alloy or a pure metal, as described above. The controlled current is preferably in the range of from about 1.5 μA to about 10 μA, more preferably in the range from about 1.5 μA to about 6 μA, still more preferably in the range from about 1.5 μA to about 5 μA, most preferably about 4.9 μA. This range is to ensure that only ions are produced by the current and not larger particles. The capped voltage is preferably less than about 1.2 V and is more preferably about 0.92 V; however, other voltages can be used. This battery-powered device has a single female plug connector that supplies current to the anode, typically to the stylet as described above, which functions as the anode, with the metallic alloy and a female snap connector for the cathode EKG-type pad for return of the supplied electrical current to complete the circuit. Alternatively, the stylet can have a direct wire connection to the metal within the stylet. The battery-powered device attaches to a standard EKG electrode. The battery-powered device has the ability to adjust its constant current output based on input from the therapist as described above. The battery-powered device also has the capability of wireless communication with a smartphone, employing NFC connectivity as described above. The battery-powered device also has the capability of wireless communication via a cellular phone system to download or upload data or firmware as described above. The battery-powered device also has the capability of providing different current levels, voltage levels, waveforms, or oscillating or pulsing signals as described above.

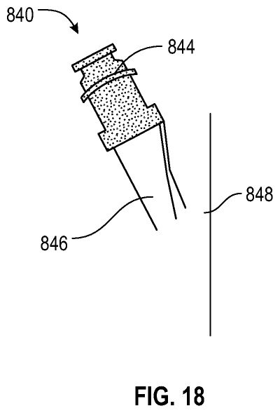

In alternatives of the present invention employing a stylet, such as, but not limited to those described in FIG. 14 or FIG. 15, the device can further comprise a needleless injector stylet so that fluids such as heparin can be injected through the catheter and stylet installation to flush the stylet and reduce fibrin buildup for longer administration times and/or where patients have a high vitamin D concentration in the blood. The needleless injector stylet is shown in FIG. 18.

In FIG. 18, the needleless injector stylet 840 has a proximal end 842 for connection to a fluid-containing device such as is commonly used for fluid supply and injection and a distal end 844 for insertion into a vein 846. The vein 846 is not part of the needleless injector stylet.

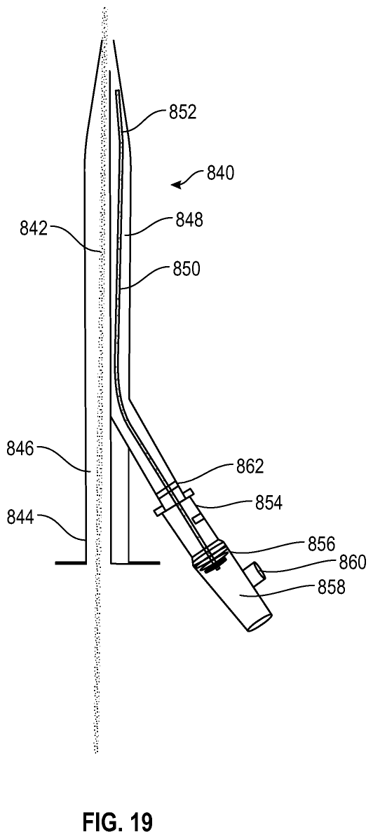

Still another embodiment of a device according to the present invention is shown in FIG. 19, which is a diagram of yet another alternative embodiment of a device according to the present invention that incorporates a dual-lumen or multi-lumen catheter for fluid delivery with a secondary lumen, an injection side port, and a hemostatic seal to prevent the backflow of blood from the secondary lumen of the dual-lumen or multi-lumen catheter.

In the device of FIG. 19, the device employs a dual-lumen or multi-lumen catheter wherein the primary lumen of the dual-lumen or multi-lumen catheter is to supply intravenous fluids and a secondary lumen of the dual-lumen or multi-lumen catheter is to house and incorporate the stylet that is connected to the wire comprised of a metal or metallic alloy as described above for the sustained release of ions generated by voltage provided by a power supply as described above. The stylet is built into the catheter and is held into place via a red pull tab or removable spacer, that once removed, allows the user of the device to move the stylet forward, exposing the wire comprised of a metal or metallic alloy that, once connected to a power supply as described above, provides a consistent, sustained release of ions for treatment of bacterial, viral, fungal, or protozoan infections, activation of the immune response, or providing a zone of inhibition around a catheter placement to protect against the development of microbial biofilm or infection from the puncture site. This alternative provides a design that is more consistent, safer, and more effective than existing catheters with coatings that contain antimicrobial metals for antimicrobial protection. The keys to the design for this alternative are: (i) the built-in stylet; (ii) the removable spacer that holds the stylet in place and that, when retracted from the distal tip, allows the introducer needle to place the catheter; and (iii) the stylet is precisely designed for the specific catheter so that, once in place, the removable spacer is removed and, once the stylet is moved forward, and locked into place, the distal tip will be exposed to enable the wire comprised of a metal or metallic alloy to be exposed for ion release. Typically, the length that is exposed is from about 1 cm (0.39 inch) to about 4 cm (about 1.56 inch). This embodiment of a device according to the present invention can be used with central catheters, mid-line catheters, PICC catheters, central venous catheters, and other catheter types.

In the device of FIG. 19, the device 840 comprises an introducer needle 842 and a dual-lumen or multi-lumen catheter 844. The dual-lumen or multi-lumen catheter 844 can be of various sizes to meet the requirements of fluid flow and insertion into a vein. The dual-lumen or multi-lumen catheter 844 has a primary lumen 846 for fluid flow. The dual-lumen or multi-lumen catheter 844 also has a secondary lumen 848 for insertion of an insulated stylet 850. The insulated stylet 850 is in electrical contact with a wire 852 comprised of a metal or metallic alloy as described above. The device 840 further comprises a removable spacer 854. The removable spacer 854 is adjacent to a connector 856 to attach the insulated stylet 850 to the catheter 844 for ion delivery. The device 840 further comprises an internal copper pin 858 for attachment to a power supply. The device 840 also further comprises an injection side port 860 to add fluids such as heparin to clean and flush the line and prevent the buildup of fibrin. The device further comprises a hemostatic seal 862 to prevent the backflow of blood from the secondary lumen. In one alternative, the pin of conductive material, typically copper, can be replaced with a heat shrink connector that incorporates a circular solder ring.

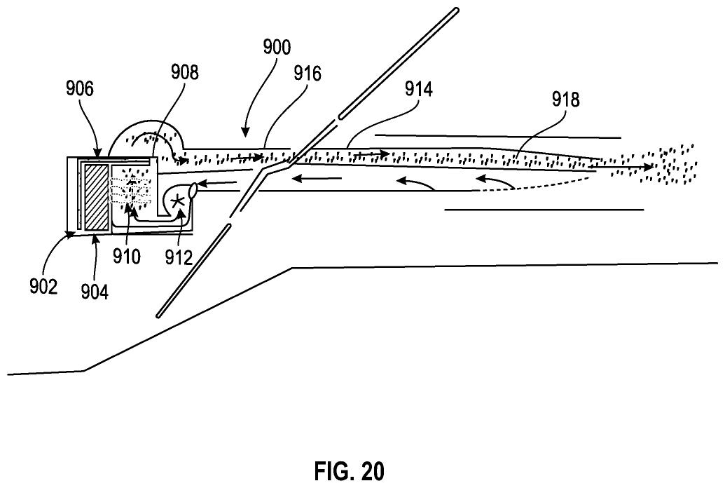

Yet another embodiment of a device according to the present invention is an extracorporeal catheter as shown in FIG. 20. FIG. 20 is a diagram of an extracorporeal catheter according to the present invention that includes a battery as a power supply, a circuit board to regulate the voltage and amperage, cathode and anode wiring with the cathode covered by an anticoagulant coating, and a pump to promote blood flow. The extracorporeal catheter is designed to be inserted into larger veins for the purposes of immune modulation and treatment of blood-borne pathogens.

In the device of FIG. 20, the device 900 includes a battery-driven power supply 902, and a circuit board 904 in operable contact with the battery-driven power supply 902 for the control of voltage and current supplied by the device. The device 900 further includes a cathode 906 covered by an anticoagulant coating 908 and anode wires 910, which may comprise from one to five wires. The device 900 further includes a pump 912 to promote blood flow. The device 900 further includes a catheter 914 with a proximal end 916 and a distal end 918 with the distal end 918 being narrower than the proximal end 916 for insertion into a vein of a subject to be treated by the device 900. The device 900 is intended to be inserted into a larger vein of the subject.

Other embodiments of the present invention include a device incorporating a dual-lumen or multi-lumen catheter where the primary function of the catheter is to deliver fluid. A range of gauges for the first lumen that delivers the fluid can be employed, depending, for example, on the volume of fluid to be delivered, the viscosity of the fluid to be delivered, and the disease or condition to be treated or prevented. A second lumen is typically 20 gauge and has a metal or metallic alloy wire built in with skived windows at its distal end to allow ionic release intravenously. This embodiment includes a built-in stylet that is integral with the catheter. In an alternative, the dual-lumen or multi-lumen catheter has a replaceable stylet rather than a built-in stylet; this alternative also has windows at the distal end of the catheter to allow the distal bare wire of the stylet to be exposed for ionic release via the skived windows built into the catheter.

In other alternative embodiments according to the present invention, a device such as an alternative of the devices described above is incorporated into a connector for an intravenous line to supply metallic ions for continuous or substantially continuous delivery.

Accordingly, one embodiment of the present invention is a device for treatment or prevention of a bacterial, viral, fungal, or protozoan infection, stimulation of the immune system, or providing a zone of inhibition around a catheter placement to protect against the development of microbial biofilm or infection from the puncture site. The device comprises:
(1) a wire comprising a metal or metallic alloy that releases ions when a low-intensity direct current is applied to the wire;
(2) an insulated covering insulating the wire, wherein the wire and the insulated covering are inserted into the bloodstream of a patient to be treated via a catheter;
(3) a male connector;
(4) a female connector in operable contact with the male connector, such that the male connector and the female connector protect components of the device and enable connection to the catheter;
(5) a conductive element selected from the group consisting of: (i) a pin of conductive material within the male connector and (ii) a heat shrink connector incorporating a circular solder ring to transmit a low-intensity direct current from a power supply connected to the device; and
(6) optionally, a side port for introduction of a medication.

The device can further comprise a power supply in electrical contact with the device.

This alternative is generally shown in FIG. 1.

In this embodiment of a device according to the present invention, the ion-producing metal wire is as described above, and can be, for example: (i) a metal or metalloid element selected from the group consisting of silver, copper, gold, platinum, iridium, zinc, palladium, and osmium; and (ii) an alloy of two or more metal or metalloid elements, each of which is selected from the group consisting of silver, copper, gold, platinum, iridium, zinc, palladium, and osmium.

Typically, in operation, the device releases from about 2×10⁹ to about 7×10¹² ions per second for the treatment of blood-borne pathogens or stimulation of the immune system as described above.

Typically, as described above, the voltage applied to generate the metallic ions is about 1.2 V or less. Preferably, the voltage applied is from about 0.5 V to about 0.92 V. As stated above, the voltage applied depends on the resistance of the tissue. Typically, the current applied to generate the metallic ions is less than about 10 μA, more typically about 6 μA or less, preferably about 4.9 μA.

Typically, the power supply typically has the ability to adjust the current based on the therapeutic application as described above. Typically, the device also has the capability of wireless communication via a cellular phone system to download and upload data, firmware, or other information as described above; alternatively, other communication routes can be used as described above. The device can be portable as described above. The device can also have the capability of providing different waveforms or of providing an oscillating or pulsing signal.

As stated above, in one alternative, the device provides constant current with a cap on voltage. In another alternative, the device provides constant voltage with a cap on current, as stated above.

Another embodiment of the present invention is another alternative of a device for treatment or prevention of a bacterial, viral, fungal, or protozoan infection, the stimulation of the immune system, or providing a zone of inhibition around a catheter placement to protect against the development of microbial biofilm or infection from the puncture site. The device comprises:
(1) a wire comprising a metal or metallic alloy that releases ions when a low-intensity direct current is applied to the wire;
(2) an insulated covering insulating the wire, wherein the wire and the insulated covering are inserted into the bloodstream of a patient to be treated via a catheter;
(3) a male connector;
(4) a female connector in operable contact with the male connector, such that the male connector and the female connector protect components of the device and enable connection to the catheter;
(5) a conductive element selected from the group consisting of: (i) a pin of conductive material within the male connector and (ii) a heat shrink connector incorporating a circular solder ring to transmit a low-intensity direct current from a power supply connected to the device;
(6) a stylet for insertion into a vein of a subject to be treated;
(7) insulation to insulate the stylet; and
(8) optionally, a side port for introduction of a medication.

The device can further comprise a power supply in electrical contact with the device.

This alternative is generally shown in FIG. 2.

In this alternative, the ion-producing metal wire is as described above.

In this alternative, the rate of release of ions for the treatment of blood-borne pathogens is as described above.

In this alternative, the voltage to generate the metallic ions is as described above.

In this alternative, the capabilities of the power supply and the capabilities of the device for communication are as described above. In this alternative, the device can be portable as described above. In this alternative, the device can provide different waveforms or can provide an oscillating or pulsing signal as described above.

As stated above, in one alternative, the device provides constant current with a cap on voltage. In another alternative, the device provides constant voltage with a cap on current, as stated above.

Another embodiment of the present invention is another alternative of a device for treatment or prevention of a bacterial, viral, fungal, or protozoan infection, the stimulation of the immune system, or providing a zone of inhibition around a catheter placement to protect against the development of microbial biofilm or infection from the puncture site. The device comprises:
(1) a wire comprising a metal or metallic alloy that releases ions when a low-intensity direct current is applied to the wire;
(2) an insulated covering that insulates a portion of the wire;
(3) a connector; and
(4) an electrode that rests on top of the skin of a patient when the insulated covering and the wire are inserted into the vein of a patient.

The device can further comprise a power supply in electrical contact with the device. Details and functioning of the power supply are as described above.

This alternative is generally shown in FIG. 3.

In this alternative, the ion-producing metal wire is as described above.

In this alternative, the rate of release of ions for the treatment of blood-borne pathogens is as described above.

In this alternative, the voltage to generate the metallic ions is as described above.

In this alternative, the capabilities of the power supply and the capabilities of the device for communication are as described above. In this alternative, the device can be portable as described above. In this alternative, the device can provide different waveforms or can provide an oscillating or pulsing signal as described above.

As stated above, in one alternative, the device provides constant current with a cap on voltage. In another alternative, the device provides constant voltage with a cap on current, as stated above.

Another embodiment of the present invention is another alternative of a device for treatment or prevention of a bacterial, viral, fungal, or protozoan infection, the stimulation of the immune system, or providing a zone of inhibition around a catheter placement to protect against the development of microbial biofilm or infection from the puncture site. The device comprises:
(1) a wire comprising a metal or metallic alloy that releases ions when a low-intensity direct current is applied to the wire;
(2) an insulating covering that insulates a portion of the wire;
(3) a domed cap covering one end of the wire;
(4) one or more skived regions of the insulating covering that expose a portion of the wire for delivery of the ions when the low-intensity direct current is applied to the wire;
(5) a male connector;
(6) a female connector in operable contact with the male connector, such that the male connector and the female connector protect components of the device and enable connection to a catheter for insertion of the device; and
(7) optionally, a side port for introduction of a medication.

This alternative is generally shown in FIG. 4. Various options for this alternative are shown in FIG. 4. In one alternative shown in FIG. 4, the length of the insulated wire extending from the proximal or originating point of the male connector can range from about 1.5 inches (3.81 cm) to about 6 inches (15.24 cm) for use with a peripheral venous electrode; from about 4 inches (10.16 cm) to about 9 inches (22.86 cm) for a central venous electrode; and from about 6 inches (15.24 cm) to about 32 inches (81.28 cm) for an electrode placed within a peripherally insulated central catheter (PICC).

In this alternative, the ion-producing metal wire is as described above.

In this alternative, the rate of release of ions for the treatment of blood-borne pathogens is as described above.

In this alternative, the voltage to generate the metallic ions is as described above.

The device can further comprise a power supply in electrical contact with the device. Details and functioning of the power supply are as described above.

In this alternative, the capabilities of the power supply and the capabilities of the device for communication are as described above. In this alternative, the device can be portable as described above. In this alternative, the device can provide different waveforms or can provide an oscillating or pulsing signal as described above.

As stated above, in one alternative, the device provides constant current with a cap on voltage. In another alternative, the device provides constant voltage with a cap on current, as stated above.

Yet another embodiment of the present invention is another alternative of a device for treatment or prevention of a bacterial, viral, fungal, or protozoan infection, the stimulation of the immune system, or providing a zone of inhibition around a catheter placement to protect against the development of microbial biofilm or infection from the puncture site. The device comprises:
(1) a wire comprising a metal or metallic alloy that releases ions when a low-intensity direct current is applied to the wire, wherein the wire is coiled or braided around a catheter for insertion of the device into the bloodstream of a patient;
(2) two cap connectors in electrical contact with the wire, wherein each cap connector is connected to a power supply connected to the device to transmit a low-intensity direct current to the device; and
(3) a power supply connected to the two cap connectors.

This alternative is generally shown in FIG. 5. Various options for this alternative are shown in FIG. 5. The arrangement of the wire in relation to the catheter can vary. The wire can be arranged spirally externally to the catheter. Alternatively, the wire can be arranged spirally internally to the catheter. In yet another alternative, the wire can be arranged in a hexagonal arrangement externally to the catheter. In still another alternative, the wire can be arranged in a hexagonal arrangement internally to the catheter. In yet another alternative, the wire can be braided externally to the catheter. In still another alternative, the wire can be braided internally to the catheter.

In this alternative, the ion-producing metal wire is as described above.

In this alternative, the rate of release of ions for the treatment of blood-borne pathogens is as described above.

In this alternative, the voltage to generate the metallic ions is as described above.

In this alternative, the capabilities of the power supply and the capabilities of the device for communication are as described above. In this alternative, the device can be portable as described above. In this alternative, the device can provide different waveforms or can provide an oscillating or pulsing signal as described above.

As stated above, in one alternative, the device provides constant current with a cap on voltage. In another alternative, the device provides constant voltage with a cap on current, as stated above.

Yet another embodiment of the device is another alternative of a device for treatment or prevention of a bacterial, viral, fungal, or protozoan infection, the stimulation of the immune system, or providing a zone of inhibition around a catheter placement to protect against the development of microbial biofilm or infection from the puncture site. The device comprises:
(1) a wire comprising a metal or metallic alloy that releases ions when a low-intensity direct current is applied to the wire;
(2) an insulated covering that covers the entire wire except for one or more cut-outs;
(3) a domed cap that seals the open end of the insulated covering; and
(4) a power supply that is connected to the device to transmit the low-intensity direct current to the device.

The power supply is as described above in order to transmit the low-intensity direct current to the device. Details and functioning of the power supply are as described above.

This alternative is generally shown in FIG. 6.

In this alternative, the ion-producing metal wire is as described above.

In this alternative, the rate of release of ions for the treatment of blood-borne pathogens is as described above.

In this alternative, the voltage to generate the metallic ions is as described above.

In this alternative, the capabilities of the power supply and the capabilities of the device for communication are as described above. In this alternative, the device can be portable as described above. In this alternative, the device can provide different waveforms or can provide an oscillating or pulsing signal as described above.

As stated above, in one alternative, the device provides constant current with a cap on voltage. In another alternative, the device provides constant voltage with a cap on current, as stated above.

Yet another embodiment of the device is another alternative of a device for treatment or prevention of a bacterial, viral, fungal, or protozoan infection, the stimulation of the immune system, or providing a zone of inhibition around a catheter placement to protect against the development of microbial biofilm or infection from the puncture site. The device comprises:
(1) a wire comprising a metal or metallic alloy that releases ions when a low-intensity direct current is applied to the wire, the wire being inserted into the vein of a patient via a catheter;
(2) an insulated covering to protect the wire;
(3) a cap connector;
(4) a male connector attached near one end of the wire;
(5) a female connector attached closer to the end of the wire to which the male connector is attached than the male connector;
(6) a pin of conductive material within the male connector to transmit a low-intensity direct current from a power supply connected to the device; and
(7) optionally, a side port for introduction of a medication.

A power supply as described above is connected to the device to transmit a low-intensity direct current to the device as described above. Details and functioning of the power supply are as described above.

In this alternative, the device is inserted within a catheter, which in turn, is inserted into the vein of a patient. This alternative is generally shown in FIG. 7.

In this alternative, the ion-producing metal wire is as described above.

In this alternative, the rate of release of ions for the treatment of blood-borne pathogens is as described above.

In this alternative, the voltage to generate the metallic ions is as described above.

In this alternative, the capabilities of the power supply and the capabilities of the device for communication are as described above. In this alternative, the device can be portable as described above. In this alternative, the device can provide different waveforms or can provide an oscillating or pulsing signal as described above.

As stated above, in one alternative, the device provides constant current with a cap on voltage. In another alternative, the device provides constant voltage with a cap on current, as stated above.

Yet another embodiment of the device is another alternative of a device for treatment or prevention of a bacterial, viral, fungal, or protozoan infection, the stimulation of the immune system, or providing a zone of inhibition around a catheter placement to protect against the development of microbial biofilm or infection from the puncture site. The device is intended for use with a PICC catheter (peripherally insulated central catheter). The device comprises:
(1) a wire comprising a metal or metallic alloy that releases ions when a low-intensity direct current is applied to the wire, the wire being inserted into the vein of a patient;
(2) an insulated covering surrounding the wire;
(3) a domed cap, wherein the insulated covering extends all the way to the domed cap;
(4) one or more cut-outs providing access for the wire to the bloodstream of a patient;
(5) a male connector;
(6) a female connector;
(7) a pin of conductive material within the male connector to transmit a low-intensity direct current from a power supply connected to the device, the pin being protected by the male connector and the female connector; and
(8) optionally, a side port for introduction of a medication.

In an alternative, the pin of conductive material, typically copper, can be replaced with a heat shrink connector that incorporates a circular solder ring as described above.

A power supply as described above is connected to the device to transmit a low-intensity direct current to the device as described above. Details and functioning of the power supply are as described above.

In this alternative, the device is inserted within a PICC catheter, which in turn, is inserted into the vein of a patient. This alternative is generally shown in FIG. 8.

In this alternative, the ion-producing metal wire is as described above.

In this alternative, the rate of release of ions for the treatment of blood-borne pathogens is as described above.

In this alternative, the voltage to generate the metallic ions is as described above.

In this alternative, the capabilities of the power supply and the capabilities of the device for communication are as described above. In this alternative, the device can be portable as described above. In this alternative, the device can provide different waveforms or can provide an oscillating or pulsing signal as described above.

As stated above, in one alternative, the device provides constant current with a cap on voltage. In another alternative, the device provides constant voltage with a cap on current, as stated above.

Yet another embodiment of the device is another alternative of a device for treatment or prevention of a bacterial, viral, fungal, or protozoan infection, the stimulation of the immune system, or providing a zone of inhibition around a catheter placement to protect against the development of microbial biofilm or infection from the puncture site. This alternative employs an auxiliary use side port catheter for dual therapy. The device comprises:
(1) an auxiliary use side port catheter;
(2) an hemostatic seal attached to the catheter to close off space around the catheter;
(3) a side port to allow the administration of an intravenous antibiotic or other medication; and
(4) a wire comprising a metal or metallic alloy that releases ions when a low-intensity direct current is applied to the wire, the wire being inserted into the vein of a patient via the catheter, wherein the inner diameter of the catheter is greater than the outer diameter of the wire to allow for both the insertion of the wire and as an auxiliary venous medication delivery port via the side port and in combination with a proximal hemostatic seal.

In this alternative, the low-intensity direct current is provided by a power supply connected to the device. Details and functioning of the power supply are as described above.

This alternative is generally shown in FIG. 9.

In this alternative, the ion-producing metal wire is as described above.

In this alternative, the rate of release of ions for the treatment of blood-borne pathogens is as described above.

In this alternative, the voltage to generate the metallic ions is as described above.

In this alternative, the capabilities of the power supply and the capabilities of the device for communication are as described above. In this alternative, the device can be portable as described above. In this alternative, the device can provide different waveforms or can provide an oscillating or pulsing signal as described above.

As stated above, in one alternative, the device provides constant current with a cap on voltage. In another alternative, the device provides constant voltage with a cap on current, as stated above.

Yet another embodiment of the device is another alternative of a device for treatment or prevention of a bacterial, viral, fungal, or protozoan infection, the stimulation of the immune system, or providing a zone of inhibition around a catheter placement to protect against the development of microbial biofilm or infection from the puncture site. The device includes a catheter with wiring in various configurations as described below.

In a first configuration of this embodiment, the device comprises:
(1) a catheter;
(2) external spiral-wound wiring surrounding the catheter, wherein the wiring comprises a metal or metallic alloy that releases ions when a low-intensity direct current is applied to the wire; and
(3) a connector connecting the external spiral-wound wiring to a power supply as described above to transmit a low-intensity direct current to the device.

In a second configuration of this embodiment, the device comprises:
(1) a catheter;
(2) straight longitudinal wiring surrounding the catheter, wherein the wiring comprises a metal or metallic alloy that releases ions when a low-intensity direct current is applied to the wire, wherein the wiring includes three to five wires that are either internally exposed, externally exposed, or both internally and externally exposed;
and (3) a connector connecting the straight longitudinal wiring to a power supply as described above to transmit a low-intensity direct current to the device.

In a third configuration of this embodiment, the device comprises:
(1) a catheter;
(2) wiring in the shape of rings at the distal end of the catheter, the proximal end of the catheter, or both the distal end and the proximal end of the catheter;
all rings can be anode, or anodes and cathodes can alternate every other ring, or the catheter can be used with an external catheter cathode, such as used in an EKG path, wherein the wiring comprises a metal or metallic alloy that releases ions when a low-intensity direct current is applied to the wire; and
(3) a connector connecting the wiring in the shape of rings to a power supply as described above to transmit a low-intensity direct current to the device.

In a fourth configuration of this embodiment, the device comprises:
(1) a catheter;
(2) wiring in a hexagonal pattern (“hex design”) external to the catheter, such as used in an EKG path; the hex design can be distal, proximal, or both, with a proximal external surface, and a distal internal or external surface, or with a proximal internal surface and a distal internal or external surface, with a cathode externally applied, such as used in an EKG path, wherein the wiring comprises a metal or metallic alloy that releases ions when a low-intensity direct current is applied to the wire; and
(3) a connector connecting the wiring in the hex design to a power supply as described above to transmit a low-intensity direct current to the device.

In a fifth configuration of this embodiment, the device comprises:
(1) a catheter;
(2) wiring with periodically exposed sections of the wire from the proximal end to the distal end of the catheter, or, alternatively, at the distal end of the catheter only, with windows from about 1 mm to about 10 mm in length and from about 35° to about 180° exposure, wherein the wiring comprises a metal or metallic alloy that releases ions when a low-intensity direct current is applied to the wire; and
(3) a connector connecting the wiring with periodically exposed sections to a power supply as described above to transmit a low-intensity direct current to the device.

These alternatives are generally shown in FIG. 10, including FIGS. 10A, 10B, 10C, 10D, 10E, 10F, 10G, and 10H.

A power supply as described above is connected to the device to transmit a low-intensity direct current to the device as described above. Details and functioning of the power supply are as described above.

In this alternative, the ion-producing metal wire is as described above.

In this alternative, the rate of release of ions for the treatment of blood-borne pathogens is as described above.

In this alternative, the voltage to generate the metallic ions is as described above.

In this alternative, the capabilities of the power supply and the capabilities of the device for communication are as described above. In this alternative, the device can be portable as described above. In this alternative, the device can provide different waveforms or can provide an oscillating or pulsing signal as described above.

As stated above, in one alternative, the device provides constant current with a cap on voltage. In another alternative, the device provides constant voltage with a cap on current, as stated above.

Yet another embodiment of the device is another alternative of a device for treatment or prevention of a bacterial, viral, fungal, or protozoan infection, the stimulation of the immune system, or providing a zone of inhibition around a catheter placement to protect against the development of microbial biofilm or infection from the puncture site. The device includes a catheter with wiring in various configurations as described below. The device can use a catheter with a single lumen. Alternatively, the device can use a catheter with multiple lumens. The catheter can be intended for peripheral, central, or PICC insertion.

The device comprises:
(1) a catheter with either a single lumen or multiple lumens;
(2) wiring implanted into the catheter with skived windows implanted into the catheter, wherein the wiring comprises a metal or metallic alloy that releases ions when a low-intensity direct current is applied to the wire; and
(3) a connector connecting the wiring with periodically to a power supply as described above to transmit a low-intensity direct current to the device.

These alternatives are generally shown in FIG. 11, including FIGS. 11A and 11B.

A power supply as described above is connected to the device to transmit a low-intensity direct current to the device as described above. Details and functioning of the power supply are as described above.

In this alternative, the ion-producing metal wire is as described above.

In this alternative, the rate of release of ions for the treatment of blood-borne pathogens is as described above.

In this alternative, the voltage to generate the metallic ions is as described above.

In this alternative, the capabilities of the power supply and the capabilities of the device for communication are as described above. In this alternative, the device can be portable as described above. In this alternative, the device can provide different waveforms or can provide an oscillating or pulsing signal as described above.

As stated above, in one alternative, the device provides constant current with a cap on voltage. In another alternative, the device provides constant voltage with a cap on current, as stated above.

Yet another embodiment of the device is another alternative of a device for treatment or prevention of a bacterial, viral, fungal, or protozoan infection, the stimulation of the immune system, or providing a zone of inhibition around a catheter placement to protect against the development of microbial biofilm or infection from the puncture site. This device is a wound care bandage or patch intended to prevent or treat infection associated with a wound.

The device comprises:
(1) a substantially planar patch or bandage having a top side and a bottom side;
(2) a coating on the bottom side of the patch or bandage, the coating including therein a first wire that comprises a metal or metallic alloy that releases ions when a low-intensity direct current is applied to the first wire;
(3) a snap connector on the top side of the patch or bandage;
(4) a second wire in electrical contact with the snap connector; and
(5) a power supply to transmit a low-intensity direct current to the device, wherein the power supply is in electrical contact with the first and second wires.

The material of the patch is typically a hydrogel. A hydrogel is a network of crosslinked polymer chains that are hydrophilic, sometimes found as a colloidal gel in which water is the dispersion medium, A three-dimensional solid results from the hydrophilic polymer chains being held together by crosslinks. The crosslinks which bond the polymers of a hydrogel fall under two general categories: physical and chemical. Physical crosslinks consist of hydrogen bonds, hydrophobic interactions, and chain entanglements (among others), Because of the inherent crosslinks, the structural integrity of the hydrogel network does not dissolve from the high concentration of water. Hydrogels are highly absorbent (they can contain over 90% water) natural or synthetic polymeric networks. Other alternatives are known in the art.

This alternative is generally shown in FIGS. 12A and 12B.

In this alternative, the ion-producing metal wire is as described above.

Details and functioning of the power supply are as described above.

In this alternative, the rate of release of ions for the treatment of blood-borne pathogens is as described above.

In this alternative, the voltage to generate the metallic ions is as described above.

In this alternative, the capabilities of the power supply and the capabilities of the device for communication are as described above. In this alternative, the device can be portable as described above. In this alternative, the device can provide different waveforms or can provide an oscillating or pulsing signal as described above.

As stated above, in one alternative, the device provides constant current with a cap on voltage. In another alternative, the device provides constant voltage with a cap on current, as stated above.

Yet another embodiment of the device is another alternative intended for incorporation into an intravenous line or other device intended to supply a fluid, such as, but not limited to, plasma, medications, nutritional supplements, or electrolytes, to a patient.

The device comprises:
(1) a hubcap that acts as a cover for the device;
(2) a cell battery acting as a power source;
(3) a printed circuit board in electrical contact with the cell battery to control the current output;
(4) a cathode wire;
(5) an anode wire;
(6) a fluid-absorbing material that is in electrical contact with the cathode wire and the anode wire to provide an electrical circuit when fluid flows through the device;
(7) a first connector;
(8) a second connector, wherein the first connector and the second connector enable placement of the device into an intravenous line to supply fluid to a patient;
(9) an anode metallic pin joining the first connector and the second connector; and
(10) a cathode metallic pin joining the first connector and the second connector.

The first and second connectors can be female or male Luer locks, intravenous hose clamps, intravenous hose connectors, or other connectors conventionally used in the art.

This alternative is generally shown in FIG. 13.

In this alternative, the ion-producing metal wire is as described above.

In this alternative, the rate of release of ions for the treatment of blood-borne pathogens, for the modulation of the immune system, or for providing a zone of inhibition around a catheter placement to fight against microbial biofilm and/or infection from the puncture site of the catheter is as described above.

In this alternative, the voltage to generate the metallic ions is as described above.

In this alternative, the capabilities of the power source and the capabilities of the device for communication are as described above. In this alternative, the device can be portable as described above. In this alternative, the device can provide different waveforms or can provide an oscillating or pulsing signal as described above.

As stated above, in one alternative, the device provides constant current with a cap on voltage. In another alternative, the device provides constant voltage with a cap on current, as stated above.

Yet another embodiment of the device is a device that is attached to a fluid source that enables the production of metallic ions without insertion of the device into an intravenous line.

The device comprises:
(1) a hubcap that acts as a cover for the device;
(2) a cell battery acting as a power source;
(3) a printed circuit board in electrical contact with the cell battery to control the current output;
(4) a cathode wire;
(5) an anode wire;
(6) a fluid-absorbing material that is in electrical contact with the cathode wire and the anode wire to provide an electrical circuit when fluid flows through the device;
(7) a solid cap;
(8) a connector;
(9) a fluid source in operable contact with the connector;
(10) an anode metallic pin in operable contact with the connector; and
(11) a cathode metallic pin in operable contact with the connector.

This alternative is generally shown in FIG. 14.

In this alternative, the ion-producing metal wire is as described above.

In this alternative, the rate of release of ions for the treatment of blood-borne pathogens is as described above.

In this alternative, the voltage to generate the metallic ions is as described above.

In this alternative, the capabilities of the power source and the capabilities of the device for communication are as described above. In this alternative, the device can be portable as described above. In this alternative, the device can provide different waveforms or can provide an oscillating or pulsing signal as described above.

As stated above, in one alternative, the device provides constant current with a cap on voltage. In another alternative, the device provides constant voltage with a cap on current, as stated above.

Yet another embodiment of the device is intended to transmit metallic ions as described above into the vein of a patient via a temporarily implanted stylet or catheter.

The device comprises:
(1) a generator attached to the skin of a patient;
(2) means for regulating the output of the generator; and
(3) an implanted stylet comprising a wire, wherein the generator is in electrical contact with the wire to transmit a low-intensity direct current through the wire and into the skin of the patient. Typically, in this device, the generator is attached to the skin of a patient to be treated and transmits the signal through the skin to the implanted stylet, which will activate the release of metallic ions.

This alternative is generally shown in FIG. 15.

In this alternative, the ion-producing metal wire is as described above.

In this alternative, the rate of release of ions for the treatment of blood-borne pathogens is as described above.

In this alternative, the voltage to generate the metallic ions is as described above.

In this alternative, the capabilities of the generator and the capabilities of the device for communication are as described above. In this alternative, the device can be portable as described above. In this alternative, the device can provide different waveforms or can provide an oscillating or pulsing signal as described above.

As stated above, in one alternative, the device provides constant current with a cap on voltage. In another alternative, the device provides constant voltage with a cap on current, as stated above.

Yet another embodiment of the present invention is a chewable gum that continuously releases silver or metallic ions to be quickly absorbed into the bloodstream via the mouth for health reasons or to provide a supplement.

This embodiment is generally shown in FIG. 16.

The embodiment comprises a wafer or layer of a chewable gum that has particles of silver or another metal as described above incorporated therein, which are released on chewing, with the hydrolytic action of saliva enabling the release of the particles so that they can be absorbed into the bloodstream where ions are released from the particles for therapeutic or immunostimulatory activity.

Yet another alternative of a device according to the present invention is a device with the cathode built directly into the catheter.

This alternative is generally shown in FIG. 17.

The device comprises:
(1) a catheter, which can be either a single-lumen catheter or a multi-lumen catheter;
(2) a wire internally run within the catheter;
(3) one to five external rings surrounding the catheter;
(4) insulated wiring wherein the wiring comprises a metal or metallic alloy that releases ions when a low-intensity direct current is applied to the wire;
(5) a connector;
(6) optionally, a port for the administration of intravenous antibiotics or other desired medications; and
(7) a wire for connection to a power supply as described above to transmit a low-intensity direct current to the device, wherein the power supply is in electrical contact with the wire.

The device can further comprise a power supply as described above. Details and functioning of the power supply are as described above.

In this alternative, the ion-producing metal wire is as described above.

In this alternative, the rate of release of ions for the treatment of blood-borne pathogens is as described above.

In this alternative, the voltage to generate the metallic ions is as described above.

In this alternative, the capabilities of the power source and the capabilities of the device for communication are as described above. In this alternative, the device can be portable as described above. In this alternative, the device can provide different waveforms or can provide an oscillating or pulsing signal as described above.

As stated above, in one alternative, the device provides constant current with a cap on voltage. In another alternative, the device provides constant voltage with a cap on current, as stated above.

Yet another alternative of a device according to the present invention is a device for treatment or prevention of a bacterial, viral, fungal, or protozoan infection or the stimulation of the immune system employing an introducer needle and a dual-lumen catheter. This alternative is generally shown in FIG. 19.

The device comprises:
(1) an introducer needle;
(2) a dual-lumen or multi-lumen or multi-lumen catheter;
(3) a primary lumen for fluid flow within the dual-lumen or multi-lumen catheter;
(4) at least one secondary lumen within the dual-lumen or multi-lumen catheter;
(5) an insulated stylet for insertion into a secondary lumen;
(6) a wire comprised of a metal or metallic alloy in electrical contact with the insulated stylet;
(7) a removable spacer;
(8) a connector adjacent to the removable spacer to attach the insulated stylet to the dual-lumen or multi-lumen catheter;
(9) an internal copper pin for attachment to a power supply;
(10) an injection side port for adding a fluid; and
(11) a hemostatic seal to prevent the backflow of blood from the secondary lumen.

A power supply as described above is connected to the device to transmit a low-intensity direct current to the device as described above. Details and functioning of the power supply are as described above.

In this alternative, the ion-producing metal wire is as described above.

In this alternative, the rate of release of ions for the treatment of blood-borne pathogens is as described above.

In this alternative, the voltage to generate the metallic ions is as described above.

In this alternative, the capabilities of the power supply and the capabilities of the device for communication are as described above. In this alternative, the device can be portable as described above. In this alternative, the device can provide different waveforms or can provide an oscillating or pulsing signal as described above.

As stated above, in one alternative, the device provides constant current with a cap on voltage. In another alternative, the device provides constant voltage with a cap on current, as stated above.

This alternative of a device according to the present invention can be used with a catheter selected from the group consisting of a central catheter, a mid-line catheter, a PICC catheter, and a central venous catheter. As stated above, a dual-lumen or multi-lumen catheter can be used; when a multi-lumen catheter is used, more than one secondary lumen can be included in the catheter.

Another embodiment of the present invention is a needleless injector stylet. The needleless injector stylet can be used in conjunction with a device according to the present invention employing a stylet. This device is shown generally in FIG. 18. The needleless injector stylet comprises:
(1) a proximal end for connection to a fluid-containing device; and
(2) a distal end for insertion into a vein.

Yet another alternative of a device according to the present invention is a device for treatment or prevention of a bacterial, viral, fungal, or protozoan infection or the stimulation of the immune system employing an extracorporeal catheter. This alternative is generally shown in FIG. 20. The extracorporeal catheter comprises:
(1) a catheter for insertion into a vein, the catheter having a proximal end and a distal end;
(2) a battery-driven power supply;
(3) a circuit board in operable contact with the battery-driven power supply for the control of voltage and current supplied by the device;
(4) a cathode;
(5) an anticoagulant coating covering the cathode;
(6) ion-producing anode wires, which comprise from one to five wires comprising a metal or metallic alloy; and
(7) a pump to promote blood flow.

In this alternative, the ion-producing metal wires are as described above.

In this alternative, the rate of release of ions for the treatment of blood-borne pathogens is as described above.

In this alternative, the voltage to generate the metallic ions is as described above.

Yet another aspect of the present invention is a method of treating a blood-borne pathogenic infection.

In general, this method comprises:
(1) placing a device according to the present invention as described above into operable contact with a subject in need of treatment of a blood-borne pathogenic infection such that the device can provide metallic ions to the bloodstream of the subject; and
(2) causing the device to release metallic ions to the bloodstream of the subject to treat the infection.

Typically, the device is placed into operable contact with the subject by placing a wire of the device into a vein of the subject so that the wire releases metallic ions into the bloodstream of the patient. As used herein, the term “operable contact” refers to contact sufficient to provide sufficient current and voltage to the site of operation of the device so that sufficient ions are released to carry out the function of the device. However, as described above, other arrangements are possible. For example, the device can be placed in-line into an intravenous line so that the device releases metallic ions into fluid being delivered to the subject by the intravenous line such that the metallic ions reach the bloodstream of the patient. Alternatively, the device can be placed in-line in another fluid source so that the device releases metallic ions into the fluid source such that the metallic ions reach the bloodstream of the patient.

In this method, the voltage and current employed to release the metallic ions are as described above.

In this method, the rate of release of ions is as described above.

In this method, the mechanisms for adjustment of current and voltage and of the waveforms is as described above.

Methods according to the present invention can be used to treat infections caused by bacteria, viruses, fungi, and protozoa.

When the method is used to treat an infection caused by bacteria, the bacterial infection can be, but is not limited to, an infection caused by Staphylococcus aureus, Staphylococcus epidermidis, Bacillus cereus, Enterococcus faecalis, Enterococcus faecium, Listeria monocytogenes, Streptococcus pneumoniae, Streptococcus pyogenes, Streptococcus agalactiae, Escherichia coli, Klebsiella pneumoniae, Haemophilus influenzae, Pseudomonas aeruginosa, Acetinobacter baumannii, Neisseria meningitidis, Bacteroides fragilis, Bacillus anthracis, Yersinia pestis, Francisella tularensis, Brucella abortus, or Mycobacterium tuberculosis. A particularly significant infection is methicillin-resistant Staphylococcus aureus (MRSA). Another particularly significant infection is tuberculosis, typically caused by Mycobacterium tuberculosis, or, in some cases, by Mycobacterium bovis.

When the method is used to treat an infection caused by viruses, the viral infection can be, but is not limited to, an infection caused by human immunodeficiency virus (HIV), hepatitis A virus, hepatitis B virus, hepatitis C virus, Ebola virus, Zika virus, dengue virus, West Nile virus, dengue virus, or severe acute respiratory syndrome coronavirus 2.

When the method is used to treat an infection caused by fungi, the fungal infection can be, but is not limited to, candidiasis, aspergillosis, blastomycosis, coccidioidomycosis, infection by Cryptococcus neoformans, or infection by Cryptococcus gattii. When the fungal infection is candidiasis, the candidiasis can be caused by Candida auris, but, alternatively, can be caused by other strains of Candida.

When the method is used to treat an infection caused by protozoa, the protozoan infection can be, but is not limited to, amoebiasis, malaria, trypanosomiasis, Chagas disease, leishmaniasis, toxoplasmosis, and cryptosporidiosis. A particularly significant infection is malaria, typically caused by Plasmodium falciparum; malaria can also be caused by Plasmodium vivax, Plasmodium ovale, or Plasmodium malariae.

In another alternative, the method further comprises administration of a therapeutically effective quantity of an antimicrobial agent. The antimicrobial agent can be selected from the group consisting of an antibacterial agent, an antiviral agent, an antifungal agent, and an antiprotozoal agent, depending on the type of infection in the patient. More than one suitable antimicrobial agent can be administered.

When the antimicrobial agent is an antibacterial agent, the selection of the particular antibacterial agent depends on the particular bacterium infecting the patient, as well as other factors such as the severity of the infection, the age, weight, and sex of the patient, pharmacokinetic factors such as the liver function and kidney function of the patient, other drugs being administered to the patient, the immune function of the patient, the toxicity of the antibacterial agent administered, any sensitivities or allergic reactions to the particular antibacterial agent or the class of antibacterial agents being considered, as well as other factors known in the art and considered by those skilled in the art of prescribing and administering antibacterial agents. One of the factors to be considered in determining suitable antibacterial agents is whether the bacterium infecting the patient is Gram-negative or Gram-positive. Several classes of antibacterial agents have been designed to target Gram-negative bacteria, including aminopenicillins, ureidopenicillins, cephalosporins, beta-lactam-beta-lactamase inhibitor combinations, folate antagonists, quinolones, and carbapenems. The drugs that specifically target Gram-negative organisms include aminoglycosides, monobactams, and ciprofloxacin. The drugs that specifically target Gram-positive organisms include vancomycin, teicoplanin, quinupristin/dalfopristin, oxazolidinones, daptomycin, telavancin, and ceftaroline.

The following antibiotics are effective against methicillin-resistant Staphylococcus aureus (MRSA): vancomycin, teicoplanin, linezolid, daptomycin, trimethoprim/sulfamethoxazole, doxycycline, ceftobiprole, ceftaroline, clindamycin, dalbavancin, fusidic acid, mupirocin, omadacycline, oritavancin, tedizolid, telavancin, and tigecycline.

The following antibiotics and classes of antibiotics are effective against Pseudomonas aeruginosa: aminoglycosides, including kanamycin A, amikacin, tobramycin, dibekacin, gentamicin, sisomicin, netilmicin, neomycin B, neomycin C, paromomycin, streptomycin, and plazomicin; carbapenems, including thienamycin, imipenem, meropenem, ertapenem, doripenem, panipenem (typically administered with betamiprom), biapenem, tebipenem, razupenem, lenapenem, and tomopenem; ceftazidime; cefepime; ceftobiprole; ceftolozane (typically administered with tazobactam); fluoroquinolones, including oxolinic acid, rosoxacin, ciprofloxacin, fleroxacin, lomefloxacin, nadifloxacin, norfloxacin, ofloxacin, pefloxacin, rufloxacin, balofloxacin, grepafloxacin, levofloxacin, pazufloxacin, sparfloxacin, temafloxacin, clinafloxacin, gatifloxacin, moxifloxacin, sitafloxacin, prulifloxacin, besifloxacin, delafloxacin, and ozenoxacin; piperacillin (typically administered with tazobactam); ticarcillin; and clavulanic acid.

The following antibiotics and classes of antibiotics are effective against vancomycin-resistant Enterococcus: linezolid; streptogramins, including quinupristin, dalfopristin, pristinamycin, and virginiamycin; tigecycline; and daptomycin.

When the antimicrobial agent is an antiviral agent, the selection of the particular antiviral agent depends on the particular virus infecting the patient, as well as other factors such as the severity of the infection, the age, weight, and sex of the patient, pharmacokinetic factors such as the liver function and kidney function of the patient, other drugs being administered to the patient, the immune function of the patient, the toxicity of the antiviral agent administered, any sensitivities or allergic reactions to the particular antiviral agent or the class of antiviral agents being considered, whether the virus is a DNA virus or an RNA virus, whether the predominant form of the genome of the virus is single-stranded or double-stranded, the life cycle of the virus, particularly with respect to whether the genome of the virus can become integrated into the DNA of the cells of the patient, as well as other factors known in the art and considered by those skilled in the art of prescribing and administering antiviral agents. Additionally, as stated above, it is known in the art that many antiviral agents have a relatively limited spectrum of action as compared with the relatively broad spectra of action for many antibacterial antibiotics such as penicillins or azithromycin because of the structural and biological diversity of the viruses. Additionally, as stated above, it must be taken into account that some RNA viruses, such as the retroviruses, can become integrated in the form of DNA in the genome of the individuals that they infect, while other RNA viruses, such as severe acute respiratory syndrome coronavirus 2, lack this capacity.

Antiviral agents include, but are not limited to, the following agents: abacavir, acyclovir, adefovir, amantadine, amprenavir, umifenovir, atazanavir, baloxavir marboxil, bictegravir, emtricitabine, tenofovir alafenamide, boceprevir, cidofovir, cobicistat, lamivudine, zidovudine, daclatasvir, sofosbuvir, ribavirin, darunavir, delavirdine, didanosine, docosanol, dolutegravir, doravirine, edoxudine, efavirenz, elvitegravir, enfuvirtide, entecavir, etravirine, famciclovir, fomivirsen, fosamprenivir, foscarnet, ganciclovir, ibacitabine, ibalizumab, idoxuridine, indinavir, letermovir, lopinavir, loviride, maraviroc, methisazone, moroxydine, nelfinavir, nitazoxanide, ritonavir, oseltamivir, penciclovir, peramivir, pleconaril, raltegravir, remdesivir, rilpivirine, rimantadine, saquinavir, simprevir, stavudine, telaprevir, telbivudine, tipranavir, trifluridine, tromantadine, valaciclovir, valganciclovir, vicriviroc, vidarabine, viramidine, zalcitabine, and zanamivir.

When the antimicrobial agent is an antifungal agent, the selection of the particular antifungal agent depends on the particular fungus infecting the patient, as well as other factors such as the severity of the infection, the age, weight, and sex of the patient, pharmacokinetic factors such as the liver function and kidney function of the patient, other drugs being administered to the patient, the immune function of the patient, the toxicity of the antifungal agent administered, and any sensitivities or allergic reactions to the particular antifungal agent or the class of antifungal agents being considered, as well as other factors known in the art and considered by those skilled in the art of prescribing and administering antifungal agents.

Antifungal agents include the following: amphotericin B, candicidin, filipin, hamycin, natamycin, nystatin, bifonazole, butoconazole, clotrimazole, econazole, fenticonazole, isoconazole, ketoconazole, luticonazole, miconazole, omoconazole, oxiconazole, sertaconazole, sulconazole, tioconazole, albazonazole, efinaconazole, epoxiconazole, fluconazole, isavuconazole, itraconazole, posaconazole, propiconazole, ravuconazole, terconazole, voriconazole, abafungin, amorolfine, butenafine, naftifine, terbinafine, anidulafungin, caspofungin, micafungin, ciclopirox, 5-fluorocytosine, griseofulvin, tolnaftate, undecylenic acid, triacetin, orotomide, miltefosine, nikkomycin, piroctone olamine, and clioquinol.

Antiprotozoal agents include the following: eflornithine, furazolidone, chloroquine, hydroxychloroquine, melarsoprol, metronidazole, nifursemizone, nitazoxanide, ornidazole, paromomycin sulfate, pentamidine, pyrimethamine, quinapyramine, ronidazole, tinidazole, amodiaquine, proguanil, sulfonamides, mefloquine, atovaquone, primaquine, artemisinin, artemether, artesunate, dihydroartemisinin, arteether, halofantrine, lumefantrine, doxycycline, and clindamycin; these agents include agents effective against malaria.

Another aspect of the invention is a method of using a device according to the present invention as described above to stimulate the production of natural killer (NK) cells. NK cells are cytotoxic lymphocytes critical to the innate immune system; they provide rapid responses to virus-infected cells, acting at around 3 days after infection, and respond to tumor formation. NK cells are unique, however, as they have the ability to recognize and kill stressed cells in the absence of antibodies and MHC, allowing for a much faster immune reaction. The cells were named “natural killers” because they do not require activation to kill cells that are missing “self” markers of MHC class 1. This role is especially important because harmful cells that are missing MHC I markers cannot be detected and destroyed by other immune cells, such as T lymphocyte cells. NK cells can be recognized by their CD56⁺CD3⁻ phenotype.

NKs are known to play a role in tumor immunosurveillance by directly inducing the death of tumor cells (NKs act as cytolytic effector lymphocytes), even in the absence of surface adhesion molecules and antigenic peptides. This role of NK cells is critical to immune success particularly because T cells are unable to recognize pathogens in the absence of surface antigens. Tumor cell detection results in activation of NK cells and consequent cytokine production and release. If tumor cells do not cause inflammation, they will also be regarded as self and will not induce a T cell response. A number of cytokines are produced by NKs, including tumor necrosis factor α (TNFα), IFNγ, and interleukin 10 (IL-10). TNFα and IL-10 act as proinflammatory and immunosuppressors, respectively. The activation of NK cells and subsequent production of cytolytic effector cells impacts macrophages, dendritic cells, and neutrophils, which subsequently enables antigen-specific T and B cell responses. Instead of acting via antigen-specific receptors, lysis of tumor cells by NK cells is mediated by alternative receptors, including NKG2D, NKp44, NKp46, NKp30, and DNAM. NKG2D is a disulfide-linked homodimer which recognizes a number of ligands, including ULBP and MICA, which are typically expressed on tumor cells.

The activation of NK cells by methods according to the present invention can be used to treat malignancies at an early stage, particularly breast cancer. For the treatment of breast cancer, the activation of NK cells can be used together with other methods of treatment, including other immunotherapeutic methods, surgery, radiation, and chemotherapy. In methods according to the present invention, including methods for the activation of NK cells, means to stimulate the immune system can involve the electric field created by the generator and transmitted via the catheter or stylet activating calcium influx and efflux channels on lymphocytes, which, in turn, activates signaling in the immune system.

In general, this method comprises:
(1) placing a device according to the present invention as described above into operable contact with a subject in need of stimulating the production of natural killer cells such that the device can provide metallic ions to the bloodstream of the subject; and
(2) causing the device to release metallic ions to the bloodstream of the subject to stimulate the production of natural killer cells.

Yet another aspect of the present invention is a method for stimulating the immune system comprising the steps of:
(1) placing a device according to the present invention as described above into operable contact with a subject in need of stimulating the immune system such that the device can provide metallic ions to the bloodstream of the subject; and
(2) causing the device to release metallic ions to the bloodstream of the subject to stimulate the immune system.

In methods according to the present invention, the methods can further comprise the steps of:
(1) subsequent to the initiation of administration of treatment, determining the results of the treatment by detecting or determining the quantity or concentration of the blood-borne pathogen remaining in the blood of the subject; and
(2) if necessary, adjusting the treatment by altering the quantity of ions administered, the current, the voltage, or the waveform used, or, if an additional antibacterial, antiviral, or antifungal agent is administered, altering a factor selected from the group consisting of the identity of the additional agent being administered, the dosage of the additional agent, the frequency of administration of the additional agent, the duration of administration of the additional agent, the route of administration of the additional agent, or another factor associated with the administration of the additional agent.

Typically, the quantity or concentration of the blood-borne pathogen is detected or determined by conventional methods known in the art, such as, but not limited to, immunological assays for an antigen associated with the blood-borne pathogen, PCR assays or other nucleic acid-based assays for the genome of the blood-borne pathogen, culture methods for bacteria, histological methods based on staining of bacteria, or other conventional methods.

The activation of NK cells by methods according to the present invention as described can particularly be used to treat malignancies at an early stage, particularly breast cancer. For the treatment of breast cancer, the activation of NK cells can be used together with other methods of treatment, including other immunotherapeutic methods, surgery, radiation, and chemotherapy. Such additional methods of treatment are known in the art.

Another method according to the present invention employing devices as described above is use of these devices for extracorporeal or intravenous ion release to clean the blood of a patient in a manner similar to that of an apheresis unit.

The invention is illustrated by the following Examples. These Examples are included for illustrative purposes only, and are not intended to limit the invention.

### EXAMPLES

### Example 1

### Treatment of Dengue Fever

Dengue fever is a mosquito-borne viral disease caused by an RNA virus of the family Flaviviridae, genus Flavivirus. The virus causing dengue fever is related to yellow fever virus, West Nile virus, Zika virus, St. Louis encephalitis virus, and viruses causing other diseases. Dengue fever is endemic in many tropical regions and causes about 390 million infections per year, with about 500,000 of those infected requiring hospitalization, resulting in about 40,000 deaths. Due to climate change and increased mobility, the occurrence of dengue fever has been rapidly increasing in more temperate regions such as Europe and the southern United States.

A device according to the present invention was used to treat dengue fever. The device employed a 20 gauge×10 cm mid-line peripherally inserted catheter (PICC) with an inserted stylet as described above. The device used a silver/copper alloy as the metal or alloy. For the treatment, the rate of release of ions was about 2.5×10¹² per second. The voltage used was 0.92 V. The current used was 2.5 μA. The duration of treatment was 4 hours.

The results are shown in Table 1.

TABLE 1
Dengue Fever Trial Data

| | | | | TEMP | | PLATLETS | | | HEMATOCRIT | | |
|---|---|---|---|---|---|---|---|---|---|---|---|
| | Patient | Age | Sex | Before | After | Before | After | [symbol] | Before | After | [symbol] |
| ORIGINAL | P1 | [symbol] | M | [symbol] | [symbol] | 60,000 | [symbol] | 160,000 | 41 | 40 | 38 |
| TRIAL | P2 | [symbol] | M | Elevated | Normal | 80,000 | [symbol] | [symbol] | 40 | 40 | [symbol] |
| | P3 | [symbol] | F | Elevated | Normal | 100,000 | 145,000 | 170,000 | [symbol] | [symbol] | [symbol] |
| | P4 | [symbol] | F | Elevated | Normal | 55,000 | | 198,000 | 41 | | [symbol] |
| | P5 | [symbol] | F | Elevated | Normal | 75,000 | 110,000 | 165,000 | 36 | 37 | [symbol] |
| | P6 | [symbol] | M | Elevated | Normal | 100,000 | 140,000 | 175,000 | 47 | [symbol] | 42 |
| | P7 | [symbol] | F | Elevated | Normal | 60,000 | 100,000 | 155,000 | 32 | [symbol] | [symbol] |
| | P8 | [symbol] | F | Elevated | Normal | 100,000 | [symbol] | 198,000 | [symbol] | [symbol] | [symbol] |
| | P9 | [symbol] | F | Elevated | Normal | 70,000 | 105,000 | 150,000 | 35 | [symbol] | [symbol] |
| | P10 | [symbol] | F | Elevated | Normal | 115,000 | 150,000 | 160,000 | 40 | 40 | 50 |
| | P11 | [symbol] | M | Elevated | Normal | 100,000 | 140,000 | 210,000 | 58 | 45 | 56 |
| | P12 | [symbol] | M | Elevated | Normal | 40,000 | 80,000 | 150,000 | 35 | 30 | [symbol] |
| | P13 | [symbol] | M | Elevated | Normal | 14,000 | 80,000 | 199,000 | [symbol] | 42 | 48.7 |
| | P14 | 34 | F | Elevated | Normal | 105,000 | 145,000 | 205,000 | 40 | [symbol] | 46 |

| | | | | LEUCOCYTES | | | LYMPHOCYTES | | | HEMOGLOBIN | | |
|---|---|---|---|---|---|---|---|---|---|---|---|---|
| | Patient | Age | Sex | Before | After | [symbol] | Before | After | [symbol] | Before | After | [symbol] |
| ORIGINAL | P1 | [symbol] | M | [symbol] | [symbol] | 7.1 | 45 | 40 | [symbol] | [symbol] | 12.1 | 13.5 |
| TRIAL | P2 | [symbol] | M | 5 | 5.5 | 6.8 | 30 | [symbol] | 22 | [symbol] | [symbol] | 14 |
| | P3 | [symbol] | F | 7 | 7 | [symbol] | 42 | 41 | [symbol] | [symbol] | 12.1 | 13 |
| | P4 | [symbol] | F | 9.5 | | [symbol] | 44 | | [symbol] | [symbol] | [symbol] | [symbol] |
| | P5 | [symbol] | F | 7 | 5.5 | [symbol] | 40 | 35 | 19 | [symbol] | [symbol] | [symbol] |
| | P6 | [symbol] | M | 9.5 | 9 | 8 | 47 | 40 | 34 | 12.8 | [symbol] | 14 |
| | P7 | [symbol] | F | 10 | 8.5 | [symbol] | 43 | [symbol] | [symbol] | [symbol] | 11.5 | 11.8 |
| | P8 | [symbol] | F | [symbol] | 9 | 8.1 | 40 | 38 | 33 | [symbol] | 11.5 | 13.5 |
| | P9 | [symbol] | F | [symbol] | 7.5 | [symbol] | [symbol] | 30 | 30 | [symbol] | [symbol] | [symbol] |
| | P10 | [symbol] | F | 8.2 | [symbol] | 3.4 | 50 | [symbol] | 20 | [symbol] | [symbol] | 12.1 |
| | P11 | [symbol] | M | 8 | 8 | [symbol] | [symbol] | [symbol] | [symbol] | [symbol] | [symbol] | 14 |
| | P12 | [symbol] | M | [symbol] | 4 | 3.2 | [symbol] | 19 | 19 | 11 | [symbol] | 12 |
| | P13 | [symbol] | M | [symbol] | 5 | [symbol] | 40 | 40 | [symbol] | 12.3 | [symbol] | 14 |
| | P14 | 34 | F | [symbol] | 7 | 5.4 | [symbol] | 42 | 38 | [symbol] | [symbol] | [symbol] |

[symbol]

indicates data missing or illegible when filed

The results of Table 1 indicate effective treatment of dengue fever by the device described above. The temperature of the patients fell to normal, the platelet count returned to normal, the lymphocyte count also returned to normal, as an elevated lymphocyte count is evidence of an active infection and the drop of the lymphocyte count indicated the clearing of the infection, and the hematocrit increased. These clinical improvements were maintained for 8 weeks or more.

### Example 2

### Treatment of Various Diseases by Devices According to the Present Invention

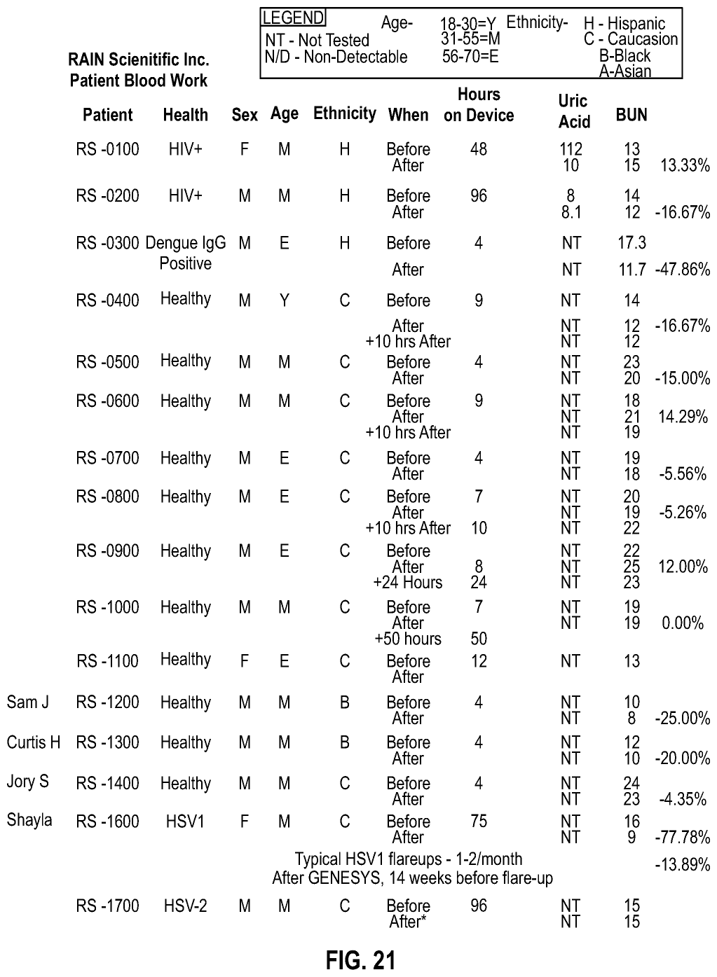

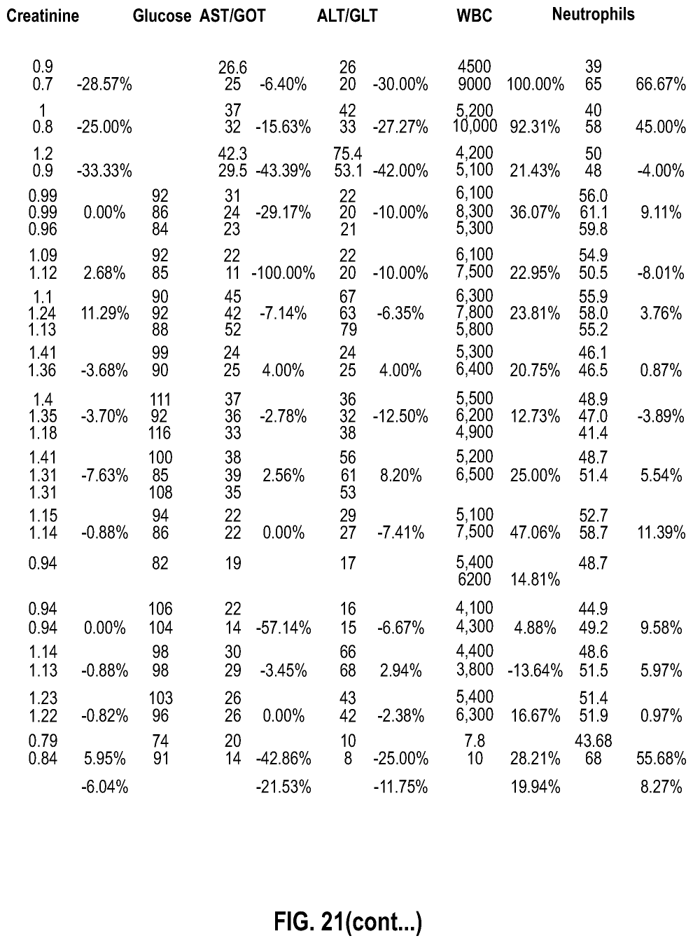

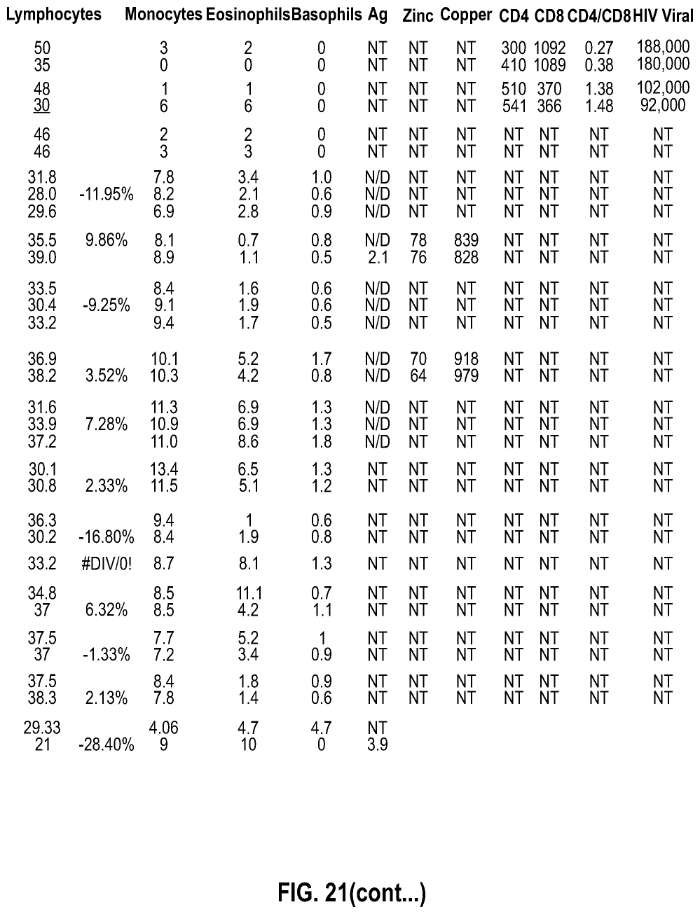

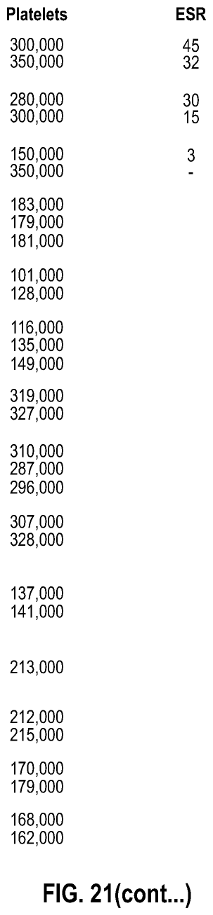

The treatment of various diseases by devices according to the present invention is shown in FIG. 21. The diseases treatable by devices according to the present invention as shown in FIG. 21 include HIV, dengue fever, infection with HSV-1, and infection with HSV-2.

In FIG. 21, “BUN” is blood urea nitrogen; “AST/GOT” is aspartate aminotransferase, a measure of liver function (an elevated value can mean liver damage); “ALT/GLT” is alanine aminotransferase, another measure of liver function (an elevated value can mean liver damage); “WBC” is white blood cell (leukocyte) count (an elevated value can mean the presence of an active infection); and “ESR” is erythrocyte sedimentation rate (an elevated value is an indication of inflammation, which can be due to infection or other causes such as an autoimmune disease).

For the results for dengue and HIV shown in FIG. 21, the device employed a 20 gauge×10 cm mid-line peripherally inserted catheter (PICC) with an inserted stylet as described above. The device used a silver/copper alloy as the metal or alloy. For the treatment, the rate of release of ions was about 2.5×10¹² per second. The voltage used was 0.92 V. The current used was 2.5 μA. The duration of treatment was 4 hours for dengue and either 24 hours or 72 hours for HIV.

For the treatment of infection with HSV-1 or infection with HSV-2 shown in FIG. 21, the device employed a 20 gauge×10 cm mid-line peripherally inserted catheter (PICC) with an inserted stylet as described above. The device used a silver/copper alloy as the metal or alloy. For the treatment, the rate of release of ions was about 5.5×10¹² per second. The voltage used was 0.89 V. The current used was 4.9 μA.

The results of Example 2 show effective treatment of HIV, dengue fever, infection with HSV-1, and infection with HSV-2.

Specific results from Example 2 for a dengue fever patient are shown in Table 2.

TABLE 2

H. Dengue Patient
RS-0300 (4 hours)

| | Before | After - +10 hours |
|---|---|---|
| BUN | 17.3 | 11.7 |
| CREA | 1.2 | 0.9 |
| BIT | 1.1 | 0.8 |
| BID | 0.9 | 0.2 |
| GOT (AST) | 43.3 | 29.5 |
| GPT (ALT) | 75.4 | 53.1 |
| ALK | 152 | 93 |
| HB | 12.3 | 13.0 |
| HCT | 37.0 | 39.0 |
| WBC | 4,200 | 5,100 |
| B | 0 | 0 |
| S | 50 | 48 |
| L | 46 | 46 |
| M | 2 | 3 |
| E | 2 | 3 |
| Platelets | 150,000 | 350,000 |

In Table 2, “BIT” is bilirubin total, “BID” is bilirubin direct, and “ALK” is alkaline phosphatase. Abnormally high levels of bilirubin can indicate different types of liver or bile duct problems and may be associated with the presence of hemolysis as bilirubin is produced during the normal breakdown of red blood cells. Alkaline phosphate concentration is (another measure of liver damage; elevated values can indicate liver damage.

Table 3 shows a comparison of platelet value in patients with dengue fever after treatment with the NANDI system versus the alternative known as GENESYS. For the NANDI system, the device employed a 20 gauge×10 cm mid-line peripherally inserted catheter (PICC) with an inserted stylet as described above. The device used a silver/copper alloy as the metal or alloy. For the treatment, the rate of release of ions was about 2.5×10¹² per second. The voltage used was 0.92 V. The current used was 2.5 μA. For the GENESYS system, the device employed a 20 gauge×10 cm mid-line peripherally inserted catheter (PICC) with an inserted stylet as described above. The device used a silver/copper alloy as the metal or alloy. For the treatment, the rate of release of ions was about 5.5×10¹² per second. The voltage used was 0.89 V. The current used was 4.9 μA.

TABLE 3
Pathogen - DENV (Dengue Fever)

| | | | |
|---|---|---|---|
| Patient | Before Platelets | After Platelets | # Days |

NANDI

| | | | |
|---|---|---|---|
| 1 | 60000 | 90000 | 4 |
| 2 | 80000 | 110000 | 4 |
| 3 | 100000 | 145000 | 4 |
| 4 | 55000 | 90000 | 5 |
| 5 | 75000 | 100000 | 4 |
| 6 | 100000 | 140000 | 4 |
| 7 | 60000 | 100000 | 4 |
| 8 | 100000 | 130000 | 4 |
| 9 | 70000 | 105000 | 4 |
| 10 | 115000 | 150000 | 3 |
| 11 | 100000 | 140000 | 3 |
| 12 | 40000 | 80000 | 3 |
| 13 | 14000 | 80000 | 4 |
| 14 | 105000 | 145000 | 3 |
| | 76714 | 114643 | 3.79 |
| | | 149.44% | |

GENESYS

| | | | |
|---|---|---|---|
| 1 | 7614 | 350000 | 1 |
| | | 233.33% | 1 |

Summation: NANDI took nearly four days to achieve a 150% increase in blood platelets, whereas improved GENESYS took only one day to achieve 233% increase in blood platelets

### Example 3

### Improvement in Liver and Renal Function and Leukocyte Count

Improvement in liver and renal function by devices according to the present invention is shown in Table 4.

TABLE 4

| | | | | | | Hours | Uric | KIDNEY | | | LIVER | |
|---|---|---|---|---|---|---|---|---|---|---|---|---|
| Patient | Health | Sex | Age | Ethnicity | When | on Device | Acid | BUN | Creatinine | Glucose | AST/GOT | ALT/GLT |
| RS-1200 | Healthy | M | 61 | Caucasian | Before - Nov. 1, 2021 | 164 | | 21 | 1.33 | 81 | 25 | 35 |
| | | | | | After - Nov. 8, 2022 | | | 20 | 1.42 | 105 | 24 | 28 |
| | | | | | | | CHANGE | [symbol] | [symbol] | | [symbol] | −20.00% |
| RS-1400 | HSV1 | F | 38 | Caucasian | Before - Nov. 1, 2021 | 75 | | 16 | 0.79 | 74 | 20 | 10 |
| | | | | | After - Nov. 7, 2021 | | | 9 | 0.84 | 91 | 14 | 8 |
| | | | | | (post 72 hours) | | | | | | | |
| | | | | | Took device | | CHANGE | [symbol] | [symbol] | | −30.00% | −20.00% |
| | | | | | off Nov. 5, 2021 | | | | | | | |

[symbol]

indicates data missing or illegible when filed

The data in Table 4 shows that treatment with a device according to the present invention can improve kidney and liver function in a healthy patient as well as in a patient infected with HSV-1.

Over a total of 12 patients, BUN was lowered by an average of 13.89% and creatine was lowered by an average of 6.4%. This shows an improvement in kidney function. For these patients, AST was lowered by an average of 21.53% and ALT was lowered by an average of 11.75%. This shows an improvement in liver function.

Additionally, for 12 healthy patients, there was a 19.94% increase in white blood cell count. This shows an increase in resistance to infection.

For the results shown in Table 4, the device employed a 20 gauge×10 cm mid-line peripherally inserted catheter (PICC) with an inserted stylet as described above. The device used a silver/copper alloy as the metal or alloy. For the treatment, the rate of release of ions was about 5.5×10¹² per second. The voltage used was 0.89 V. The current used was 4.9 μA. The time of treatment is as shown in Table 4.

### Example 4

### Treatment of HIV

Results for the treatment of HIV with a device according to the present invention are shown in Table 5.

TABLE 5

| | (2) HIV patients, were not on | | | |
|---|---|---|---|---|
| | any antiretroviral medication | | | |
| | RS-0100 (24 hours) | | RS-0200 (96 hours) | |
| | Before | After | Before | After |
| HIV Viral (Qn) | 188,000 | 180,000 | 102,000 | 92,000 |
| CD4 | 300 | 410 | 510 | 541 |
| BUN | 13.0 | 15.0 | 14.0 | 12.0 |
| CREA | 0.9 | 0.7 | 1.0 | 0.8 |
| Uric Acid | 11.2 | 10.0 | 8.0 | 8.1 |
| BIL | 0.5 | 0.5 | 0.8 | 0.6 |
| BID | 0.2 | 0.2 | 0.2 | 0.2 |
| GOT | 26.6 | 25.0 | 37.0 | 32.0 |
| GPT | 26.0 | 20.0 | 42.0 | 33.0 |
| Hemoglobin | 11.0 | 11.6 | 13.3 | 13.6 |
| Hematocrit | 33.0 | 35.0 | 40.0 | 41.0 |
| Neutrophils | 39.0 | 65.0 | 40.0 | 58.0 |
| Lymphocytes | 50.0 | 35.0 | 48.0 | 30.0 |
| WBC | 4,500 | 9,000 | 5,200 | 10,000 |
| Platelets | 300,000 | 350,000 | 280,000 | 300,000 |
| ESR | 45.0 | 32.0 | 30.0 | 15.0 |

The device used for the results in Table 5 employed a 20 gauge×10 cm mid-line peripherally inserted catheter (PICC) with an inserted stylet as described above. The device used a silver/copper alloy as the metal or alloy. For the treatment, the rate of release of ions was about 2.5×10¹² per second. The voltage used was 0.92 V. The current used was 2.5 μA. The time of treatment is as stated in Table 5.

The results are on patients who were not on antiviral medications. The results indicate that a device according to the present invention was effective at less than 72 hours of treatment to reduce viral load and increase the concentration of CD4⁺ cells.

### Advantages of the Invention

Devices and methods according to the present invention provide an improved way to treat blood-borne pathogenic infections, particularly bacterial, viral, and fungal infections, employing a new mechanism for treatment that does not depend on the specificity of conventional antibacterial, antiviral, or antifungal therapeutic agents. The devices and methods according to the present application are of broad therapeutic application and are well-tolerated and do not induce significant side effects. They can be used together with other methods of treatment, including, but not limited to, conventional antibacterial, antiviral, or antifungal therapeutic agents, as well as methods such as the use of ventilators to treat infections affecting the lungs, such as pneumonia or infection with severe acute respiratory syndrome coronavirus 2, as well as the use of steroids such as dexamethasone to prevent uncontrolled response of the immune system (so-called “cytokine storms”) which can complicate treatment of viral diseases. Devices and methods according to the present invention can further act to stimulate the immune system in conjunction with inhibiting the growth of pathogens.

Devices according to the present invention possess industrial applicability as devices useful for medical treatment. Methods according to the present invention possess industrial applicability for the preparation of a medicament to treat diseases and conditions described herein, and also encompass the use of devices according to the present invention for treatment of diseases and conditions described herein, including but not limited to bacterial, viral, and fungal infections.

With respect to ranges of values, the invention encompasses each intervening value between the upper and lower limits of the range to at least a tenth of the lower limit's unit, unless the context clearly indicates otherwise. Moreover, the invention encompasses any other stated intervening values and ranges including either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test this invention.

The publications and patents disclosed herein are provided solely for their disclosure prior to the filing date of the present application. Nothing herein is to be construed as an admission that the present invention is not entitled to antedate such publication by virtue of prior invention. Further the dates of publication provided may be different from the actual publication dates which may need to be independently confirmed.

All the publications cited are incorporated herein by reference in their entireties, including all published patents, patent applications, and literature references, as well as those publications that have been incorporated in those published documents. However, to the extent that any publication incorporated herein by reference refers to information to be published, applicants do not admit that any such information published after the filing date of this application to be prior art.

As used in this specification and in the appended claims, the singular forms include the plural forms. For example, the terms “a,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” or equivalent terminology shall be read expansively and without limitation. As used herein, the transitional phrase “comprising” also encompasses the transitional phrases “consisting essentially of” and “consisting of” unless the transitional phrases with a narrower meaning are expressly excluded. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

1.-24. (canceled)

25. A device for treatment or prevention of a bacterial, viral, fungal, or protozoan infection, stimulation of the immune system, or providing a wall of inhibition around a catheter placement to protect against the development of microbial biofilm or infection from the puncture site comprising:
(a) a wire comprising a metal or metallic alloy that releases ions when a low-intensity direct current is applied to the wire;
(b) an insulated covering that insulates a portion of the wire;
(c) a connector; and
(d) an electrode that rests on top of the skin of a patient when the insulated covering and the wire are inserted into the vein of a patient.

26. The device of claim 25 wherein the wire comprising a metal or metallic alloy is selected from the group consisting of:
(a) an alloy of gold and silver in which the alloy comprises 70% or more of gold and 30% or less of silver;
(b) an alloy of gold and silver in which the alloy comprises 70% or more of silver and 30% or less of gold;
(c) an alloy of gold, silver, and copper in which the alloy comprises 70% or more of gold and a total of 30% or less of silver and copper;
(d) an alloy of silver, copper, and gold in which the alloy comprises 70% or more of silver and a total of 30% or less of copper and gold;
(e) an alloy of silver, copper, and gold in which the alloy comprises 70% or more of copper and a total of 30% or less of silver and gold;
(f) an alloy of silver, copper, and gold in which the alloy comprises between 1% and 99% each of gold, silver, and copper;
(g) an alloy of gold and one or more of silver, copper, platinum, iridium, and zinc in which the alloy comprises 70% or more of gold, and a total of 30% or less of silver, copper, platinum, iridium, or zinc;
(h) an alloy of silver and one or more of gold, copper, platinum, iridium, and zinc in which the alloy comprises 70% or more of silver and a total of 30% or less of gold, copper, platinum, iridium, or zinc;
(i) an alloy of copper and one or more of gold, silver, platinum, iridium, and zinc in which the alloy comprises 70% or more of copper and a total of 30% or less of gold, silver, platinum, iridium, or zinc;
(j) an alloy of platinum and one or more of gold, silver, copper, iridium, and zinc in which the alloy comprises 70% or more of platinum and a total of 30% or less of gold, silver, copper, iridium, or zinc;
(k) an alloy of iridium and one or more of gold, silver, copper, platinum, and zinc in which the alloy comprises 70% or more of iridium and a total of 30% or less of gold, silver, copper, platinum, or zinc; and
(l) an alloy of zinc and one or more of gold, silver, copper, platinum, and iridium in which the alloy comprises 70% or more of zinc and a total of 30% or less of gold, silver, copper, platinum, or iridium.

27. The device of claim 25 wherein the device releases from about 2×10⁹ to about 7×10¹² ions per second in operation.

28. The device of claim 25 wherein the device employs a voltage of about 1.2 V or less.

29. (canceled)

30. The device of claim 25 wherein the device employs a current of less than about 10 μA.

31.-32. (canceled)

33. The device of claim 25 further comprising a power supply in electrical contact with the device.

34. The device of claim 33 wherein the power supply in electrical contact with the device has the ability to adjust the current based on the therapeutic application of the device.

35. The device of claim 25 wherein the device provides constant current with a cap on voltage.

36. The device of claim 25 wherein the device provides constant voltage with a cap on current.

37.-49. (canceled)

50. A device for treatment or prevention of a bacterial, viral, fungal, or protozoan infection, stimulation of the immune system, or providing a wall of inhibition around a catheter placement to protect against the development of microbial biofilm or infection from the puncture site comprising:
(a) a wire comprising a metal or metallic alloy that releases ions when a low-intensity direct current is applied to the wire, wherein the wire is coiled or braided around a catheter for insertion of the device into the bloodstream of a patient; and
(b) two cap connectors in electrical contact with the wire, wherein each cap connector is connected to a power supply connected to the device to transmit a low-intensity direct current to the device; and
(c) a power supply connected to the two cap connectors.

51. The device of claim 50 wherein the wire is arranged relative to the catheter in an alternative selected from the group consisting of:
(a) spirally externally to the catheter;
(b) spirally internally to the catheter;
(c) hexagonally externally to the catheter;
(d) hexagonally internally to the catheter;
(e) braided externally to the catheter; and
(f) braided internally to the catheter.

52. The device of claim 50 wherein the wire comprising a metal or metallic alloy is selected from the group consisting of:
(a) an alloy of gold and silver in which the alloy comprises 70% or more of gold and 30% or less of silver;
(b) an alloy of gold and silver in which the alloy comprises 70% or more of silver and 30% or less of gold;
(c) an alloy of gold, silver, and copper in which the alloy comprises 70% or more of gold and a total of 30% or less of silver and copper;
(d) an alloy of silver, copper, and gold in which the alloy comprises 70% or more of silver and a total of 30% or less of copper and gold;
(e) an alloy of silver, copper, and gold in which the alloy comprises 70% or more of copper and a total of 30% or less of silver and gold;
(f) an alloy of silver, copper, and gold in which the alloy comprises between 1% and 99% each of gold, silver, and copper;
(g) an alloy of gold and one or more of silver, copper, platinum, iridium, and zinc in which the alloy comprises 70% or more of gold, and a total of 30% or less of silver, copper, platinum, iridium, or zinc;
(h) an alloy of silver and one or more of gold, copper, platinum, iridium, and zinc in which the alloy comprises 70% or more of silver and a total of 30% or less of gold, copper, platinum, iridium, or zinc;
(i) an alloy of copper and one or more of gold, silver, platinum, iridium, and zinc in which the alloy comprises 70% or more of copper and a total of 30% or less of gold, silver, platinum, iridium, or zinc;
(j) an alloy of platinum and one or more of gold, silver, copper, iridium, and zinc in which the alloy comprises 70% or more of platinum and a total of 30% or less of gold, silver, copper, iridium, or zinc;
(k) an alloy of iridium and one or more of gold, silver, copper, platinum, and zinc in which the alloy comprises 70% or more of iridium and a total of 30% or less of gold, silver, copper, platinum, or zinc; and
(l) an alloy of zinc and one or more of gold, silver, copper, platinum, and iridium in which the alloy comprises 70% or more of zinc and a total of 30% or less of gold, silver, copper, platinum, or iridium.

53. The device of claim 50 wherein the device releases from about 2×10⁹ to about 7×10¹² ions per second in operation.

54. The device of claim 50 wherein the device employs a voltage of about 1.2 V or less.

55. (canceled)

56. The device of claim 50 wherein the device employs a current of less than about 10 μA.

57.-58. (canceled)

59. The device of claim 50 wherein the power supply in electrical contact with the device has the ability to adjust the current based on the therapeutic application of the device.

60. The device of claim 50 wherein the device provides constant current with a cap on voltage.

61.-96. (canceled)

97. A device for treatment or prevention of a bacterial, viral, fungal, or protozoan infection, stimulation of the immune system, or providing a wall of inhibition around a catheter placement to protect against the development of microbial biofilm or infection from the puncture site comprising:
(a) an open lumen catheter;
(b) an hemostatic seal attached to the catheter to close off space around the catheter;
(c) a side port to allow the administration of an intravenous antibiotic or other medication; and
(d) a wire comprising a metal or metallic alloy that releases ions when a low-intensity direct current is applied to the wire, the wire being inserted into the vein of a patient via the catheter, wherein the inner diameter of the catheter lumen is greater than the outer diameter of the wire to allow for both the insertion of the wire and as an auxiliary venous medication port via the side port and in combination with a proximal hemostatic seal.

98. The device of claim 97 wherein the wire comprising a metal or metallic alloy is selected from the group consisting of:
(a) an alloy of gold and silver in which the alloy comprises 70% or more of gold and 30% or less of silver;
(b) an alloy of gold and silver in which the alloy comprises 70% or more of silver and 30% or less of gold;
(c) an alloy of gold, silver, and copper in which the alloy comprises 70% or more of gold and a total of 30% or less of silver and copper;
(d) an alloy of silver, copper, and gold in which the alloy comprises 70% or more of silver and a total of 30% or less of copper and gold;
(e) an alloy of silver, copper, and gold in which the alloy comprises 70% or more of copper and a total of 30% or less of silver and gold;
(f) an alloy of silver, copper, and gold in which the alloy comprises between 1% and 99% each of gold, silver, and copper;
(g) an alloy of gold and one or more of silver, copper, platinum, iridium, and zinc in which the alloy comprises 70% or more of gold, and a total of 30% or less of silver, copper, platinum, iridium, or zinc;
(h) an alloy of silver and one or more of gold, copper, platinum, iridium, and zinc in which the alloy comprises 70% or more of silver and a total of 30% or less of gold, copper, platinum, iridium, or zinc;
(i) an alloy of copper and one or more of gold, silver, platinum, iridium, and zinc in which the alloy comprises 70% or more of copper and a total of 30% or less of gold, silver, platinum, iridium, or zinc;
(j) an alloy of platinum and one or more of gold, silver, copper, iridium, and zinc in which the alloy comprises 70% or more of platinum and a total of 30% or less of gold, silver, copper, iridium, or zinc;
(k) an alloy of iridium and one or more of gold, silver, copper, platinum, and zinc in which the alloy comprises 70% or more of iridium and a total of 30% or less of gold, silver, copper, platinum, or zinc; and
(l) an alloy of zinc and one or more of gold, silver, copper, platinum, and iridium in which the alloy comprises 70% or more of zinc and a total of 30% or less of gold, silver, copper, platinum, or iridium.

99. The device of claim 97 wherein the device releases from about 2×10⁹ to about 7×10¹² ions per second in operation.

100. The device of claim 97 wherein the device employs a voltage of about 1.2 V or less.

101. (canceled)

102. The device of claim 97 wherein the device employs a current of less than about 10 μA.

103.-104. (canceled)

105. The device of claim 97 further comprising a power supply in electrical contact with the device.

106. The device of claim 105 wherein the power supply has the ability to adjust the constant current based on the therapeutic application of the device.

107. The device of claim 97 wherein the device provides constant current with a cap on voltage.

108. The device of claim 97 wherein the device provides constant voltage with a cap on current.

109. A device for treatment or prevention of a bacterial, viral, fungal, or protozoan infection, stimulation of the immune system, or providing a wall of inhibition around a catheter placement to protect against the development of microbial biofilm or infection from the puncture site comprising:
(a) a catheter;
(b) wiring surrounding the catheter, the configuration of the wiring selected from the group consisting of:
(i) external spiral-wound wiring surrounding the catheter;
(ii) straight longitudinal wiring surrounding the catheter;
(iii) wiring in the shape of rings at the distal end of the catheter, the proximal end of the catheter, or both the distal end and the proximal end of the catheter,
(iv) wiring in a hexagonal pattern external to the catheter; and
(v) wiring with periodically exposed sections of the wire from the proximal end to the distal end of the catheter, or, alternatively, at the distal end of the catheter only;
wherein the wiring comprises a metal or metallic alloy that releases ions when a low-intensity direct current is applied to the wire; and
(c) a connector connecting the wiring in the hex design to a power supply to transmit a low-intensity direct current to the device.

110. The device of claim 109 wherein the wiring surrounding the catheter is in a configuration of external spiral-wound wiring surrounding the catheter.

111. The device of claim 109 wherein the wiring surrounding the catheter is in a configuration of straight longitudinal wiring surrounding the catheter.

112. The device of claim 109 wherein the wiring surrounding the catheter is in a configuration of wiring in the shape of rings at the distal end of the catheter, the proximal end of the catheter, or both the distal end and the proximal end of the catheter.

113. The device of claim 109 wherein the wiring surrounding the catheter is in a configuration of wiring in a hexagonal pattern external to the catheter.

114. The device of claim 109 wherein the wiring surrounding the catheter is in a configuration of wiring with periodically exposed sections of the wire from the proximal end to the distal end of the catheter, or, alternatively, at the distal end of the catheter only.

115. The device of claim 109 wherein the wire comprising a metal or metallic alloy is selected from the group consisting of:
(a) an alloy of gold and silver in which the alloy comprises 70% or more of gold and 30% or less of silver;
(b) an alloy of gold and silver in which the alloy comprises 70% or more of silver and 30% or less of gold;
(c) an alloy of gold, silver, and copper in which the alloy comprises 70% or more of gold and a total of 30% or less of silver and copper;
(d) an alloy of silver, copper, and gold in which the alloy comprises 70% or more of silver and a total of 30% or less of copper and gold;
(e) an alloy of silver, copper, and gold in which the alloy comprises 70% or more of copper and a total of 30% or less of silver and gold;
(f) an alloy of silver, copper, and gold in which the alloy comprises between 1% and 99% each of gold, silver, and copper;
(g) an alloy of gold and one or more of silver, copper, platinum, iridium, and zinc in which the alloy comprises 70% or more of gold, and a total of 30% or less of silver, copper, platinum, iridium, or zinc;
(h) an alloy of silver and one or more of gold, copper, platinum, iridium, and zinc in which the alloy comprises 70% or more of silver and a total of 30% or less of gold, copper, platinum, iridium, or zinc;
(i) an alloy of copper and one or more of gold, silver, platinum, iridium, and zinc in which the alloy comprises 70% or more of copper and a total of 30% or less of gold, silver, platinum, iridium, or zinc;
(j) an alloy of platinum and one or more of gold, silver, copper, iridium, and zinc in which the alloy comprises 70% or more of platinum and a total of 30% or less of gold, silver, copper, iridium, or zinc;
(k) an alloy of iridium and one or more of gold, silver, copper, platinum, and zinc in which the alloy comprises 70% or more of iridium and a total of 30% or less of gold, silver, copper, platinum, or zinc; and
(l) an alloy of zinc and one or more of gold, silver, copper, platinum, and iridium in which the alloy comprises 70% or more of zinc and a total of 30% or less of gold, silver, copper, platinum, or iridium.

116. The device of claim 109 wherein the device releases from about 2×10⁹ to about 7×10¹² ions per second in operation.

117. The device of claim 109 wherein the device employs a voltage of about 1.2 V or less.

118. (canceled)

119. The device of claim 109 wherein the device employs a current of less than about 10 μA.

120.-121. (canceled)

122. The device of claim 109 further comprising a power supply in electrical contact with the device.

123. The device of claim 122 wherein the power supply has the ability to adjust the current based on the therapeutic application of the device.

124. The device of claim 109 wherein the device provides constant current with a cap on voltage.

125. The device of claim 109 wherein the device provides constant voltage with a cap on current.

126.-267. (canceled)
